(19) United States
(12) Patent Application Publication
(10) Pub. No.: US 2026/0077050 A1
(43) Pub. Date: Mar. 19, 2026

(54) PROTAC DEGRADERS OF SARS-COV-2 MAIN PROTEASE

(71) Applicant: THE TEXAS A&M UNIVERSITY SYSTEM, College Station, TX (US)

(72) Inventors: Shiqing Xu, College Station, TX (US); Wenshe Liu, College Station, TX (US); Benjamin Neuman, College Station, TX (US); Yugendar R. Alugubelli, College Station, TX (US); Kaustav Khatua, College Station, TX (US)

(21) Appl. No.: 18/889,910

(22) Filed: Sep. 19, 2024

(51) Int. Cl. A61K 47/54; A61K 47/55

(52) U.S. Cl. CPC A61K 47/545; A61K 47/55

(57) ABSTRACT

The present disclosure relates to certain molecules, pharmaceutical compositions containing them, and methods of using them to treat viral infections.

### STATEMENT OF GOVERNMENT RIGHTS

This invention was made with government support under AI164088 awarded by the National Institutes of Health. The Government has certain rights in the invention.

### REFERENCE TO AN ELECTRONICALLY SUBMITTED SEQUENCE LISTING

The contents of the electronic sequence listing, named 414033_Sequences.xml was created Sep. 19, 2024, and is 8 kilobytes, is herein incorporated by reference in its entirety.

### TECHNICAL FIELD

The present disclosure relates to certain molecules, pharmaceutical compositions containing them, and methods of using them to treat viral infections.

### BACKGROUND

Coronaviruses (CoVs) are a family of single-stranded, positive-sense RNA viruses with a wide-ranging ability to infect mammals including humans and birds. They are responsible for a spectrum of diseases, including respiratory, hepatic, enteric, and neurologic diseases. Prior to 2003, only a few CoVs such as HCoV-229E and HCoV-OC436-7 were recognized as human pathogens. However, within a span of just two decades, the world witnessed the emergence of three major severe respiratory disease outbreaks: the severe acute respiratory syndrome coronavirus (SARS-CoV) in 2003, Middle East respiratory syndrome coronavirus (MERS-CoV) in 2012, and SARS-CoV-2 in 2019. These outbreaks swiftly evolved into significant public health crises. In particular, the ongoing coronavirus disease 2019 (COVID-19) pandemic caused by SARS-CoV-2 has evolved into one of the most formidable public health challenges in human history. According to statistics released from the World Health Organization (WHO) on Aug. 23, 2023, the numbers of confirmed cases and deaths of COVID-19 worldwide have exceeded 770 million and 6.9 million, respectively. The persistent recurrence of CoV pandemics over the past two decades has led scientists to believe that new CoV strains will periodically emerge in human populations. This phenomenon is attributed to various factors, including the high prevalence and widespread distribution of CoVs, their substantial genetic diversity, and the ever-increasing close human-animal interactions in modem society. Given the ongoing COVID-19 pandemic and the potential for future CoV outbreaks, it is imperative to prioritize the development of small-molecule drugs that can be easily distributed as effective CoV antivirals for both treatment and prevention.

The SARS-CoV-2 genome encodes conserved replicase and structural proteins, plus several small open reading frames (ORFs) that encode accessory proteins dispensable for virus growth. ORF1ab, the largest ORF, contains overlapping open reading frames that encode polyproteins pp1ab and pp1a. These polyproteins are cleaved to yield 16 nonstructural proteins (nsps), nsp1-16, through their two viral proteases: papain-like proteinase protein (PLPro, nsp3) and 3C-like (3CLPro, nsp5) that is also called main protease (MPro, nsp5). These nsps play a pivotal role in viral transcription, replication, proteolytic processing, suppression of host immune responses and host gene expression. Notably, MPro is responsible for processing 13 out of the 16 critical nsps that are indispensable for viral replication and packaging. Furthermore, MPro exhibits a high degree of conservation among various CoVs, making it one of the most attractive drug targets for developing antivirals against COVID-19. MPro is a cysteine protease with three domains, and its active form is a homodimer comprising two protomers. Each protomer contains a Cys145-His41 catalytic dyad, where cysteine serves as the nucleophile in the proteolytic process. Consequently, the primary strategy for the development of antivirals has centered on high-affinity ligands that can bind directly to MPro, thereby inhibiting its enzymatic activities and functions. A significant milestone in MPro inhibitors was the development of nirmatrelvir, an orally available reversible covalent SARS-CoV-2 MPro inhibitor developed at Pfizer. In December 2021, the US FDA granted emergency use authorization for Paxlovid, a combination therapy comprising nirmatrelvir and ritonavir, for the treatment of COVID-19. By May 2023, Paxlovid received full FDA approval for use in high-risk adults, and has been granted conditional or emergency use authorization in more than 70 countries worldwide for combating COVID-19. While Paxlovid has generated significant excitement, it is essential to acknowledge several associated issues. These include but are not limited to the potential for serious side effects, drug interactions, the COVID-19 rebound likely due to inadequate drug exposure, and the emergence of drug resistance associated with naturally occurring mutations of SARS-CoV-2 MPro. Given these challenges, there remains a critical need for antiviral therapies, particularly those with alternative mechanisms of action that can potentially address these concerns, in the ongoing battle against SARS-CoV-2 and newly emerging CoV pathogens.

Proteolysis targeting chimera (PROTAC) represents an innovative technology for targeted protein degradation in precision medicine. These rationally designed small-molecule PROTACs are heterobifunctional compounds composed of two active ligands interconnected by a chemical linker. One of these ligands is tailored to bind specifically to a protein of interest (POI) while the other selectively engages an E3 ubiquitin ligase. The recruitment of the E3 ligase to the target protein facilitates the formation of a ternary complex, leading to the ubiquitination and ultimate degradation of the target protein. PROTACs offer numerous advantages over traditional occupancy-based inhibitors, including: (i) the ability to degrade multi-domain proteins, eliminating both enzymatic and non-enzymatic/scaffolding functions; (ii) flexibility in recruiting targets via various binding sites, even in cases where sustained inhibition is unnecessary. This enables the degradation of “undruggable” targets, using allosteric sites if needed; (iii) the potential to transform weak binders into potent degraders; (iv) a catalytic nature allowing for sub-stoichiometric activity and improved efficacy; (v) enhanced target selectivity achieved through protein-protein interactions between the targeted protein and the recruited E3 ligase; (vi) capability to overcome drug resistance stemming from target protein overexpression by degrading the full-length protein; and (vii) enhanced intracellular accumulation and target engagement. These distinctive features position PROTAC as a potential game-changing technology in drug discovery that has been widely explored for degrading various disease-causing proteins. Harnessing these advantages, antiviral PROTACs via targeted protein degradation has been considered as a promising strategy for developing next-generation antiviral drugs to combat infectious diseases. The antiviral PROTACs would improve resistance profiles to those of traditional inhibitors.

### SUMMARY

In one aspect, the disclosure provides a conjugate having a formula Q-L-P, wherein Q is a radical of a ubiquitin ligase ligand; L is a divalent linker; and P is a radical of a SARS-CoV-2 protease ligand.

In another aspect, the disclosure provides a pharmaceutical composition comprising at least a compound or composition of the disclosure, or a pharmaceutically acceptable salt thereof, and optionally one or more pharmaceutically acceptable excipients.

In yet another aspect, the disclosure provides a method of treating disease, such as a viral infection, comprising administering to a subject in need of such treatment an effective amount of a compound or composition of the disclosure, or a pharmaceutically acceptable salt thereof.

### BRIEF DESCRIPTIONS OF THE DRAWING

FIG. 1 shows a schematic design of M^{Pro} PROTAC degraders based on reversible covalent inhibitor MPI8. MPD1-6 were prepared via linking a CRBN ligand at N-terminal phenyl group at P4 site of MPI8.

FIG. 2 shows a schematic design of M^{Pro} PROTAC degraders based on reversible covalent inhibitor MPI29. MPD7-10 were prepared via linking a CRBN ligand at the N-terminal tert-butyl group of MPI29.

FIG. 3A shows representative chemical structures of M^{Pro} PROTAC degraders MPD1-MPD10.

FIG. 3B shows graphs of inhibition curves of MPD1-MPD10 on M^{Pro}. Triplicate experiments were performed for each compound. For all experiments, 20 nM M^{Pro} was incubated with an inhibitor for 30 min before 10 μM Sub3 was added. The M^{Pro}-catalyzed Sub3 hydrolysis rate was determined by measuring linear increase of product fluorescence (Ex: 336 nm/Em: 490 nm) at the initial 5 min reaction time.

FIG. 4A shows a design of M^{Pro}-eGFP fusion protein.

FIG. 4B shows representative flow cytometry analysis of the potency of MPD2 in degrading M^{Pro} in M^{Pro}-eGFP 293T stable cell line. M^{Pro}-eGFP cells were evaluated after being treated with different concentrations of MPD2 for 48 h. The percentage of positively expressed M^{Pro}-eGFP fusion protein was displayed at the bottom of the graph.

FIG. 4C shows the potency of MPD1 in degrading M^{Pro} was evaluated in M^{Pro}-eGFP 293T stable cell line by immunoblots after the cells were treated with different concentrations of MPD1 for 48 h. A representative immunoblot is shown, and 3-actin was used as a loading control in all immunoblot analyses.

FIG. 4D shows a graph of the normalized protein content in the immunoblot of FIG. 4C as mean values±s.e.m. (n=3) in the graph.

FIG. 4E shows the potency of MPD2 in degrading M^{Pro} was evaluated in M^{Pro}-eGFP 293T stable cell line by immunoblots after the cells were treated with different concentrations of MPD2 for 48 h. A representative immunoblot is shown, and β-actin was used as a loading control in all immunoblot analyses.

FIG. 4F shows a graph of the normalized protein content in the immunoblot of FIG. 4E as mean values±s.e.m. (n=3) in the graph.

FIG. 4G shows the potency of MPD3 in degrading M^{Pro} was evaluated in M^{Pro}-eGFP 293T stable cell line by immunoblots after the cells were treated with different concentrations of MPD3 for 48 h. A representative immunoblot is shown, and j-actin was used as a loading control in all immunoblot analyses.

FIG. 4H shows a graph of the normalized protein content in the immunoblot of FIG. 4G as mean values±s.e.m. (n=3) in the graph.

FIG. 5A shows a graph of cytotoxicity of MPD1 in 293T cells using the MTT assay. Dose-response curves for the tested drugs in 293T cells. Various dilutions of the drugs were applied to the 80% confluent cell monolayers and assayed after 48 h to determine the CC₅₀ (half-maximal cytotoxic concentrations). Nonlinear regression analysis of GraphPad Prism software (version 8.0) was used to calculate CC₅₀ by plotting log compounds versus normalized response (variable compounds).

FIG. 5B shows a graph of cytotoxicity of MPD2 in 293T cells using the MTT assay. Dose-response curves for the tested drugs in 293T cells. Various dilutions of the drugs were applied to the 80% confluent cell monolayers and assayed after 48 h to determine the CC₅₀ (half-maximal cytotoxic concentrations). Nonlinear regression analysis of GraphPad Prism software (version 8.0) was used to calculate CC₅₀ by plotting log compounds versus normalized response (variable compounds).

FIG. 5C shows a graph of cytotoxicity of MPD3 in 293T cells using the MTT assay. Dose-response curves for the tested drugs in 293T cells. Various dilutions of the drugs were applied to the 80% confluent cell monolayers and assayed after 48 h to determine the CC₅₀ (half-maximal cytotoxic concentrations). Nonlinear regression analysis of GraphPad Prism software (version 8.0) was used to calculate CC₅₀ by plotting log compounds versus normalized response (variable compounds).

FIG. 6A shows the time course of MPD2-mediated M^{Pro} degradation as evaluated in M^{Pro}-eGFP 293T stable cell line by immunoblots after the cells were treated with 3 μM MPD2 for various time points as indicated. PROTAC degrader MPD2 downregulates the protein levels of M^{Pro} in a time-dependent manner.

FIG. 6B shows a graph presenting the normalized protein content in the immunoblots as mean values±s.e.m. (n=3 biologically independent experiments) in the graph.

FIG. 7 shows a graph of the stability of MPD1-MPD3 in human liver microsomes.

FIG. 8A shows pretreatment with M^{Pro} ligand MPI8 or CRBN ligand Pomalidomide blocks the M^{Pro} degradation induced by MPD2. The potency of MPD2 in degrading M^{Pro} was evaluated in M^{Pro}-eGFP 293T stable cell line by immunoblots after the cells were treated with different concentrations of MPD2 for 48 h.

FIG. 8B shows a graph of the normalized protein content in the immunoblots (β-actin was used as a loading control) presented as mean values±s.e.m. (n=3 biologically independent experiments) in the titration curved graph.

FIG. 8C shows CRISPR knockout of CRBN blocks MPD2-induced M^{Pro} degradation as shown in control (CRBN-CN) and CRBN knockout (CRBN-KO) M^{Pro}-eGFP 293T cells. Immunoblots evaluated the potency of MPD2 in degrading M^{Pro} after the cells were treated with different concentrations of MPD2 for 48 h.

FIG. 8D shows a graph of the normalized protein content in the immunoblots (0-actin was used as a loading control) presented as mean values±s.e.m. (n=3 biologically independent experiments) in the titration curved graph.

FIG. 9 shows flow cytometry analysis of the potency of MPD2 in degrading M^{Pro} in pretreatments of vehicle, MPI8, CRBN ligand (Pomalidomide) in M^{Pro}-eGFP 293T stable cell line or CRISPR knockout of CRBN in M^{Pro}-eGFP 293T stable cell line. The percentage of positively expressed M^{Pro}-eGFP fusion protein was displayed on the upright top panel in every histogram.

FIG. 10A shows a representative of 3 immunoblot analyses of M^{Pro} in M^{Pro}-eGFP 293T stable cell line after they were pretreated with the proteasome inhibitor MG132 (1 μM) for 1 h, and then were treated with different concentration of MPD2 for 48 h.

FIG. 10B shows a representative of 3 immunoblot analyses of M^{Pro} in M^{Pro}-eGFP 293T stable cell line after they were pretreated with vehicle for 1 h, and then were treated with different concentration of MPD2 for 48 h.

FIG. 10C shows a graph of the normalized protein content in the immunoblots β-actin was used as a loading control) from FIG. 10A in the titration curved graph.

FIG. 10D shows a graph of the normalized protein content in the immunoblots β-actin was used as a loading control) from FIG. 10B in the titration curved graph.

FIG. 11A shows a graph of antiviral effectiveness of PROTACs against SARS-CoV-2 delta variant. A549-ACE2 cells were inoculated with 0.01 TCID50 per cell SARS-CoV-2 for 1 h, then treated with antiviral. Effect of PROTACs or nirmatrelvir (Nir) treatment on virus growth 48 h after inoculation is shown.

FIG. 11B shows antiviral effectiveness of PROTACs against SARS-CoV-2 delta variant. A549-ACE2 cells were inoculated with 0.1 TCID50 per cell SARS-CoV-2 for 1 h, then treated with antiviral. Effect of PROTACs treatment on MPro accumulation 48 h after inoculation with SARS-CoV-2 delta variant is shown. MPro in cell lysates was detected with monoclonal antibody against the 34 kDa MPro.

FIG. 12A shows a graph of antiviral activity of MPD2 against SARS-CoV-2 variants. MPD2 impedes SARS-CoV-2 strain WA.1 infection. A549-hACE2 cells were infected with WA.1 (0.1) the presence of various concentrations of MPD2. After 48 h post-infection, supernatant viral RNAs from each treatment dose were quantified and normalized to that of DMSO controls.

FIG. 12B shows a graph of antiviral activity of MPD2 against SARS-CoV-2 variants. MPD2 impedes SARS-CoV-2 strain BA.1 infection. A549-hACE2 cells were infected with BA.1 (0.3) in the presence of various concentrations of MPD2. After 48 h post-infection, supernatant viral RNAs from each treatment dose were quantified and normalized to that of DMSO controls.

FIG. 12C shows a graph of antiviral activity of MPD2 against SARS-CoV-2 variants. MPD2 impedes SARS-CoV-2 strain XBB.1.5 infection. A549-hACE2 cells were infected with XBB.1.5 (MOI 0.3) in the presence of various concentrations of MPD2. After 48 h post-infection, supernatant viral RNAs from each treatment dose were quantified and normalized to that of DMSO controls.

FIG. 12D shows an graph of MPD2 inhibition of M^{Pro} E166A mutant. A549-hACE2 cells were infected with recombinant live-attenuated Δ3678 mGFP or M^{Pro} E166A mutant and treated with MPD2 for 48 h. mGFP-positive cells from each treatment dose were quantified and normalized to that of DMSO controls.

FIG. 12E shows a table of antiviral activity of MPD2 against HCoV-229E and HCoV-OC43 by a virus yield reduction assay.

FIG. 13 shows the synergistic effect of PROTACs with nirmatrelvir and ritonavir, the components of Paxlovid, on SARS-CoV-2 growth 48 h after inoculation.

FIG. 14 shows the synergistic effect of PROTAC MPD2, 10 nM Remdesivir and 5 μM Ritonavir on SARS-CoV-2 growth 48 h after inoculation.

FIG. 15 shows the synergistic effect of PROTAC MPD2, 4.4 μM Molnupiravir and 5 μM Ritonavir on SARS-CoV-2 growth 48 h after inoculation.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Definitions

As used herein, the term “alkyl” includes a chain of carbon atoms, which is optionally branched and contains from 1 to 20 carbon atoms. It is to be further understood that in certain embodiments, alkyl may be advantageously of limited length, including C₁-C₁₂, C₁-C₁₀, C₁-C₉, C₁-C₈, C₁-C₇, C₁-C₆, and C₁-C₄, Illustratively, such particularly limited length alkyl groups, including C₁-C₈, C₁-C₇, C₁-C₆, and C₁-C₄, and the like may be referred to as “lower alkyl.” Illustrative alkyl groups include, but are not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like. Alkyl may be substituted or unsubstituted. Typical substituent groups include cycloalkyl, aryl, heteroaryl, heteroalicyclic, hydroxy, alkoxy, aryloxy, mercapto, alkylthio, arylthio, cyano, halo, carbonyl, oxo (═O), thiocarbonyl, O-carbamyl, N-carbamyl, O-thiocarbamyl, N-thiocarbamyl, C-amido, N-amido, C-carboxy, O-carboxy, —NO₂, and amino, or as described in the various embodiments provided herein. It will be understood that “alkyl” may be combined with other groups, such as those provided above, to form a functionalized alkyl. By way of example, the combination of an “alkyl” group, as described herein, with a “carboxy” group may be referred to as a “carboxyalkyl” group. Other non-limiting examples include hydroxyalkyl, aminoalkyl, and the like.

As used herein, the term “alkenyl” includes a chain of carbon atoms, which is optionally branched, and contains from 2 to 20 carbon atoms, and also includes at least one carbon-carbon double bond (i.e., C═C). It will be understood that in certain embodiments, alkenyl may be advantageously of limited length, including C₂-C₁₂, C₂-C₉, C₂-C₈, C₂-C₇, C₂-C₆, and C₂-C₄. Illustratively, such particularly limited length alkenyl groups, including C₂-C₈, C₂-C₇, C₂-C₆, and C₂-C₄ may be referred to as lower alkenyl. Alkenyl may be unsubstituted, or substituted as described for alkyl or as described in the various embodiments provided herein. Illustrative alkenyl groups include, but are not limited to, ethenyl, 1-propenyl, 2-propenyl, 1-, 2-, or 3-butenyl, and the like.

As used herein, the term “alkynyl” includes a chain of carbon atoms, which is optionally branched, and contains from 2 to 20 carbon atoms, and also includes at least one carbon-carbon triple bond (i.e., C≡C). It will be understood that in certain embodiments, alkynyl may each be advantageously of limited length, including C₂-C₁₂, C₂-C₉, C₂-C₈, C₂-C₇, C₂-C₆, and C₂-C₄. Illustratively, such particularly limited length alkynyl groups, including C₂-C₈, C₂-C₇, C₂-C₆, and C₂-C₄ may be referred to as lower alkynyl. Alkynyl may be unsubstituted, or substituted as described for alkyl or as described in the various embodiments provided herein. Illustrative alkynyl groups include, but are not limited to, ethynyl, 1-propynyl, 2-propynyl, 1-, 2-, or 3-butynyl, and the like.

As used herein, the term “aryl” refers to an all-carbon monocyclic or fused-ring polycyclic groups of 6 to 12 carbon atoms having a completely conjugated pi-electron system. It will be understood that in certain embodiments, aryl may be advantageously of limited size such as C₆-C₁₀ aryl. Illustrative aryl groups include, but are not limited to, phenyl, naphthylenyl and anthracenyl. The aryl group may be unsubstituted, or substituted as described for alkyl or as described in the various embodiments provided herein.

As used herein, the term “cycloalkyl” refers to a 3 to 15 member all-carbon monocyclic ring, including an all-carbon 5-member/6-member or 6-member/6-member fused bicyclic ring, or a multicyclic fused ring (a “fused” ring system means that each ring in the system shares an adjacent pair of carbon atoms with each other ring in the system) group, or a carbocyclic ring that is fused to another group such as a heterocyclic, such as ring 5- or 6-membered cycloalkyl fused to a 5- to 7-membered heterocyclic ring, where one or more of the rings may contain one or more double bonds but the cycloalkyl does not contain a completely conjugated pi-electron system. It will be understood that in certain embodiments, cycloalkyl may be advantageously of limited size such as C₃-C₁₂, C₃-C₉, C₃-C₈, C₃-C₆ and C₄-C₆. Cycloalkyl may be unsubstituted, or substituted as described for alkyl or as described in the various embodiments provided herein. Illustrative cycloalkyl groups include, but are not limited to, cyclopropyl, cyclobutyl, cyclopentyl, cyclopentenyl, cyclopentadienyl, cyclohexyl, cyclohexenyl, cycloheptyl, adamantyl, norbornyl, norbornenyl, 9H-fluoren-9-yl, and the like. Illustrative examples of cycloalkyl groups shown in graphical representations include the following entities, in the form of properly bonded moieties:

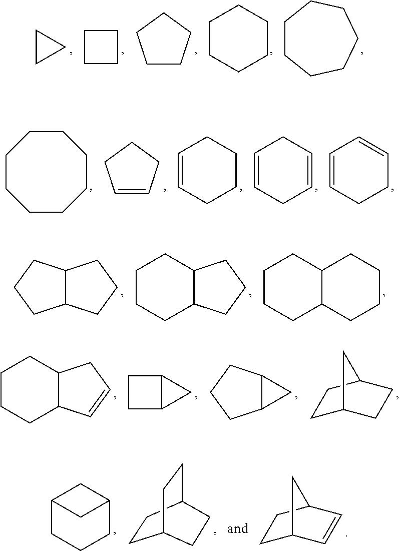

As used herein, the terms “heterocycloalkyl”, “heterocyclyl”, or “heterocycle” refers to a monocyclic or fused ring group having in the ring(s) from 3 to 12 ring atoms, in which at least one ring atom is a heteroatom, such as nitrogen, oxygen or sulfur, the remaining ring atoms being carbon atoms. Heterocycloalkyl may optionally contain 1, 2, 3 or 4 heteroatoms. A heterocycloalkyl group may be fused to another group such as another heterocycloalkyl, an aryl, or a heteroaryl group. Heterocycloalkyl may also have one of more double bonds, including double bonds to nitrogen (e.g., C═N or N═N) but does not contain a completely conjugated pi-electron system. It will be understood that in certain embodiments, heterocycloalkyl may be advantageously of limited size such as 3- to 7-membered heterocycloalkyl, 5- to 7-membered heterocycloalkyl, 3-, 4-, 5- or 6-membered heterocycloalkyl, and the like. Heterocycloalkyl may be unsubstituted, or substituted as described for alkyl or as described in the various embodiments provided herein. Illustrative heterocycloalkyl groups include, but are not limited to, oxiranyl, thianaryl, azetidinyl, oxetanyl, tetrahydrofuranyl, pyrrolidinyl, tetrahydropyranyl, piperidinyl, 1,4-dioxanyl, morpholinyl, 1,4-dithianyl, piperazinyl, oxepanyl, 3,4-dihydro-2H-pyranyl, 5,6-dihydro-2H-pyranyl, 2H-pyranyl, 1, 2, 3, 4-tetrahydropyridinyl, and the like. Illustrative examples of heterocycloalkyl groups shown in graphical representations include the following entities, in the form of properly bonded moieties:

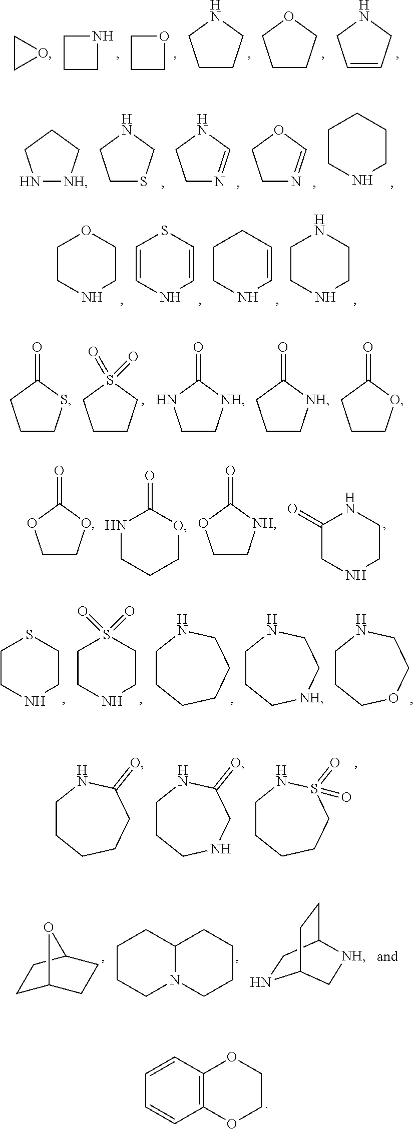

In a particular embodiment, a heterocycloalkyl group is depicted by the structure

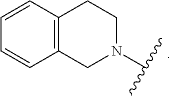 [SMILES: *N1CCc2ccccc2C1]

As used herein, the term “aryl” refers to a monocyclic or fused ring group of 5 to 10 ring atoms, and also having a completely conjugated pi-electron system. It will be understood that in certain embodiments, aryl may be advantageously of limited size such as 6- to 10-membered aryl and the like. Aryl may be unsubstituted, or substituted as described for alkyl or as described in the various embodiments provided herein. Illustrative aryl groups include, but are not limited to, phenyl and naphthyl.

As used herein, the term “heteroaryl” refers to a monocyclic or fused ring group of 5 to 10 ring atoms containing one, two, three or four ring heteroatoms selected from nitrogen, oxygen and sulfur, the remaining ring atoms being carbon atoms, and also having a completely conjugated pi-electron system. It will be understood that in certain embodiments, heteroaryl may be advantageously of limited size such as 3- to 7-membered heteroaryl, 5- to 7-membered heteroaryl, 5- to 10-membered heteroaryl and the like. Heteroaryl may be unsubstituted, or substituted as described for alkyl or as described in the various embodiments provided herein. In some embodiments, it will be understood that an atom exo to the heteroaryl ring atoms, such as an amino or a hydroxyl group, may contribute to the pi-electron system. For example, tautomeric hydroxyl-pyridine contains an —OH group, wherein the exo oxygen contributes to the pi-electron system, illustrated in the tautomeric 2-pyridone configuration.

Illustrative heteroaryl groups include, but are not limited to, pyrrolyl, furanyl, thiophenyl, imidazolyl, oxazolyl, thiazolyl, pyrazolyl, pyridinyl, pyrimidinyl, quinolinyl, isoquinolinyl, purinyl, tetrazolyl, triazinyl, pyrazinyl, tetrazinyl, quinazolinyl, quinoxalinyl, thienyl, isoxazolyl, isothiazolyl, oxadiazolyl, thiadiazolyl, triazolyl, benzimidazolyl, benzoxazolyl, benzthiazolyl, benzisoxazolyl, benzisothiazolyl and carbazoloyl, and the like. Illustrative examples of heteroaryl groups shown in graphical representations, include the following entities, in the form of properly bonded moieties:

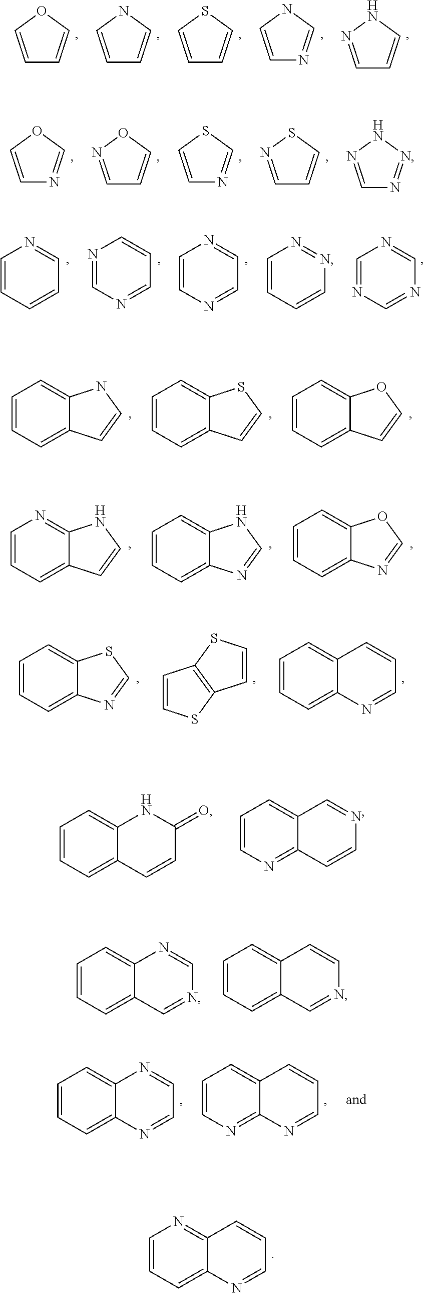

As used herein, “tautomer” refers each of two or more isomers of a compound which exist together in equilibrium, and are readily interchanged by migration of an atom or group within the molecule. In a particular embodiment, a tautomer can be either tautomer configuration of 2-pyridone, such as

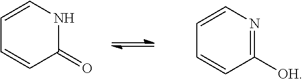 [SMILES: O=c1cccc[nH]1 ; Oc1ccccn1]

In a particular embodiment, a tautomer can be either tautomer configuration of pyrimidinedione, such as

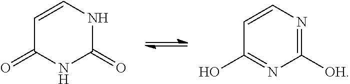 [SMILES: O=c1cc[nH]c(=O)[nH]1 ; Oc1ccnc(O)n1]

As used herein, “hydroxy” or “hydroxyl” refers to an —OH group.

As used herein, “alkoxy” refers to both an —O-(alkyl) or an —O-(unsubstituted cycloalkyl) group. Representative examples include, but are not limited to, methoxy, ethoxy, propoxy, butoxy, cyclopropyloxy, cyclobutyloxy, cyclopentyloxy, cyclohexyloxy, and the like.

As used herein, “halo” or “halogen” refers to fluorine, chlorine, bromine or iodine.

As used herein, “cyano” refers to a —CN group.

The term “oxo” represents a carbonyl oxygen. For example, a cyclopentyl substituted with oxo is cyclopentanone. In another example, a hydroxy substituted aromatic compound may exist as an oxo substituted tautomer, such as hydroxyl-pyridine and pyridinone. In a particular embodiment, a hydroxy-substituted pyridine may be represented as an oxo tautomer, such as

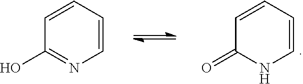 [SMILES: Oc1ccccn1 ; O=c1cccc[nH]1]

In some embodiments, a hydroxy-substituted quinoline may be represented as an oxo tautomer, such as

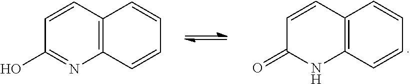 [SMILES: Oc1ccc2ccccc2n1 ; O=c1ccc2ccccc2[nH]1]

As used herein, “bond” refers to a covalent bond.

The term “substituted” means that the specified group or moiety bears one or more substituents. The term “unsubstituted” means that the specified group bears no substituents. Where the term “substituted” is used to describe a structural system, the substitution is meant to occur at any valency-allowed position on the system. In some embodiments, “substituted” means that the specified group or moiety bears one or more substituents. It will be understood that in certain embodiments, substitutions may be advantageously limited in number. In some embodiments, “substituted” means that the specified group or moiety bears one to five substituents. In other embodiments, “substituted” means that the specified group or moiety bears one, two, or three substituents. In other embodiments, “substituted” means that the specified group or moiety bears one or two substituents. In still other embodiments, “substituted” means the specified group or moiety bears one substituent.

As used herein, “optional” or “optionally” means that the subsequently described event or circumstance may but need not occur, and that the description includes instances where the event or circumstance occurs and instances in which it does not. For example, “wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl is independently optionally substituted by C₁-C₆ alkyl” means that an alkyl may be but need not be present on any of the C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl by replacement of a hydrogen atom for each alkyl group, and the description includes situations where the C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl is substituted with an alkyl group and situations where the C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl is not substituted with the alkyl group.

As used herein, “independently” means that the subsequently described event or circumstance is to be read on its own relative to other similar events or circumstances. For example, in a circumstance where several equivalent hydrogen groups are optionally substituted by another group described in the circumstance, the use of “independently optionally” means that each instance of a hydrogen atom on the group may be substituted by another group, where the groups replacing each of the hydrogen atoms may be the same or different. Or for example, where multiple groups exist all of which can be selected from a set of possibilities, the use of “independently” means that each of the groups can be selected from the set of possibilities separate from any other group, and the groups selected in the circumstance may be the same or different.

As used herein, the phrase “taken together with the atoms to which they are attached” or “combine together with the atom to which they are attached” means that, for example, two substituents (e.g., two independent R^{c} groups) attached to two separate atoms or attached to the same atom form the groups that are defined by the claim, such as

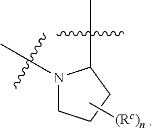

In particular, the phrase “combine together with the atoms to which they are attached” means, for example, two independent R^{c} groups on different ring atoms of

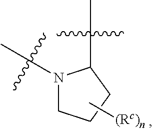

such as carbon atom “1” and carbon atom “2” respectively, as shown in the structure below form a ring with those ring atoms.

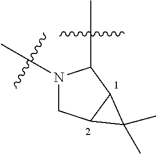 [SMILES: *C1C2C(CN1*)C2(C)C]

For example, the phrase “two R^{c} combine together with the atoms to which they are attached combine to form” used in connection with the embodiments described herein includes the compounds represented as follows:

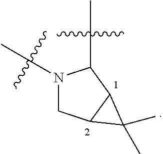 [SMILES: *C1C2C(CN1*)C2(C)C]

Alternatively, the phrase can be directed to two substituents on the same atom. For example, the phrase “two R^{f} together with the atom to which they are attached combine to form” used in connection with the embodiments described herein includes the compounds represented as follows:

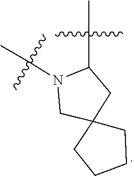 [SMILES: *C1CC2(CCCC2)CN1*]

As used herein, the term “pharmaceutically acceptable salt” refers to those salts which counter ions which may be used in pharmaceuticals. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt. Such salts include:

(1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or

(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like.

Pharmaceutically acceptable salts are well known to those skilled in the art, and any such pharmaceutically acceptable salt may be contemplated in connection with the embodiments described herein. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a compound of Formula I that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

The disclosure also relates to pharmaceutically acceptable prodrugs of the compounds of Formula I, and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula I). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

The present disclosure also relates to pharmaceutically active metabolites of compounds of Formula I, and uses of such metabolites in the methods of the disclosure. A “pharmaceutically active metabolite” means a pharmacologically active product of metabolism in the body of a compound of Formula I, or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof. For example, it will be appreciated that compounds depicted by a structural formula containing the symbol “ [symbol] ” include both stereoisomers for the carbon atom to which the symbol “ [symbol] ” is attached, specifically both the bonds “ [symbol] ” and “ [symbol] ” are encompassed by the meaning of “ [symbol] ”.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. For example, isotope-labeled compounds and salts can be used as medicaments. Further, substitution with heavier isotopes such as deuterium (i.e., ²H) may afford certain therapeutic advantages resulting from greater metabolic stability, for example increased in vivo half-life or reduced dosage requirements. For example, deuterium (²H)-labeled compounds and salts may be therapeutically useful with potential therapeutic advantages over the non-²H-labeled compounds. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

Any disubstituent referred to herein is meant to encompass the various attachment possibilities when more than one of such possibilities are allowed. For example, reference to disubstituent -A-B—, where A≠B, refers herein to such disubstituent with A attached to a first substituted member and B attached to a second substituted member, and it also refers to such disubstituent with A attached to the second substituted member and B attached to the first substituted member.

### REPRESENTATIVE EMBODIMENTS

In some embodiments, the disclosure relates to a conjugate having a formula Q-L-P, wherein Q is a radical of a ubiquitin ligase ligand; L is a divalent linker; and P is a radical of a SARS-CoV-2 protease ligand.

In some embodiments, the radical of a ubiquitin ligase ligand (Q) is an E3 ligase ligand (e.g., a cereblon (CRBN) ligand or a von Hippel-Lindau (VHL) ligand).

In some embodiments, the ubiquitin ligase ligand (Q) is of Formula II:

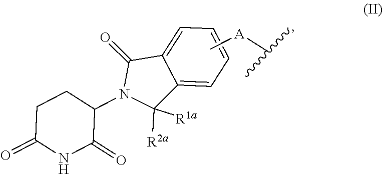

or a pharmaceutically acceptable salt thereof, wherein
A is ether, amino, amide, carbamate, carbonate, ester, ketone, sulfate, sulfonamide, sulfoxide, sulfone, sulfonate, thioester, thioether, alkoxy, urea, hydrazine, or aminoalkyl; and
each of R^{1a}, R^{2a} is H or alkyl (e.g., C₁-C₆ alkyl); or R^{1a} and R^{2a} combine with the carbon atom to which they are attached to form an oxo;
wherein “ [symbol] ” represents a point of attachment to the divalent linker (L).

In some embodiments, A is ether (e.g., —O—), amino (e.g., —NR^{3a}—, such as —N(CH₃)—), amide (e.g., —NR^{3a}C(O)—), carbamate (e.g., —NR^{3a}C(O)O—), carbonate (e.g., —OC(O)O—), ester (e.g., —C(O)O—), urea (e.g., —NR^{3a}C(O)NR^{3a}—), alkoxy (e.g., —O—C₁-C₆ alkyl), or aminoalkyl (e.g., —NR^{3a}—C₁-C₆ alkylene). In some embodiments, A is ether (e.g., —O—), amino (e.g., —NR^{3a}—, such as —NH— or —N(CH₃)—), or amide (e.g., —NR^{3a}C(O)—, such as —NHC(O)— or —N(CH₃)C(O)—). In some embodiments, A is —O—, —NCH₃—, or —NHC(O)—).

In some embodiments, R^{1a} is H or alkyl (e.g., C₁-C₆ alkyl). In some embodiments, R^{1a} is H. In some embodiments, R^{1a} is C₁-C₆ alkyl (e.g., methyl).

In some embodiments, R^{2a} is H or alkyl (e.g., C₁-C₆ alkyl). In some embodiments, R^{2a} is H. In some embodiments, R^{2a} is C₁-C₆ alkyl (e.g., methyl).

In some embodiments, R^{1a} and R^{2a} come together on the carbon atom to which they are attached to form an oxo. In some embodiments, R^{1a} is C₁-C₆ alkyl (e.g., methyl) and R^{2a} is H.

In some embodiments, each R^{3a}, when present, is independently H or alkyl (e.g., C₁-C₆ alkyl). In some embodiments, each R^{3a}, when present, is independently H or methyl.

In some embodiments, the ubiquitin ligase ligand (Q) is of Formula III:

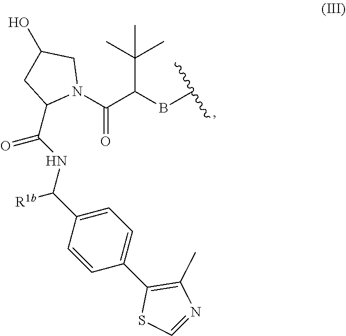

or a pharmaceutically acceptable salt thereof, wherein
B is ether, amino, amide, carbamate, carbonate, ester, ketone, sulfate, sulfonamide, sulfoxide, sulfone, sulfonate, thioester, thioether, alkoxy, urea, hydrazine, or aminoalkyl; and
R^{1b} is H or alkyl (e.g., C₁-C₆ alkyl);
wherein “ [symbol] ” represents a point of attachment to the divalent linker (L).

In some embodiments, B is ether (e.g., —O—), amino (e.g., —NR^{2b}—), amide (e.g., —NR^{2b}C(O)—), carbamate (e.g., —NR^{2b}C(O)O—), carbonate (e.g., —OC(O)O—), ester (e.g., —C(O)O—), urea (e.g., —NR^{2b}C(O)NR^{2b}—), alkoxy (e.g., —O—C₁-C₆ alkyl), or aminoalkyl (e.g., —NR^{2b}—C₁-C₆ alkyl). In some embodiments, B is amino (e.g., —NR^{2b}—), or amide (e.g., —NR^{3b}C(O)—). In some embodiments, B is —NCH₃—, or —NHC(O)—).

In some embodiments, R^{1b} is H or alkyl (e.g., C₁-C₆ alkyl). In some embodiments, each R^{1b} is H or methyl.

In some embodiments, the ubiquitin ligase ligand (Q) is

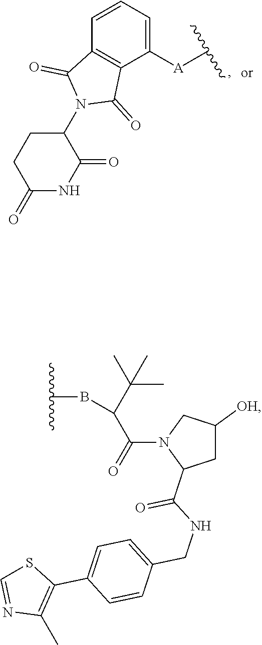

or a pharmaceutically acceptable salt thereof, wherein A and B are as defined herein.

In some embodiments, the ubiquitin ligase ligand (Q) is

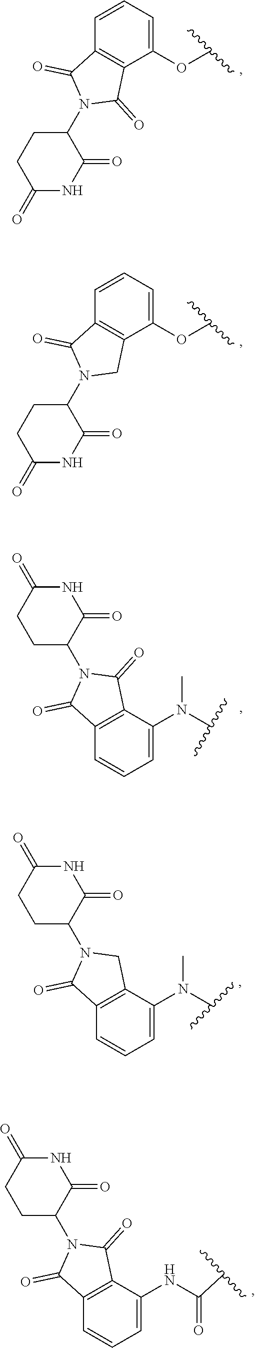 [SMILES: *Oc1cccc2c1C(=O)N(C1CCC(=O)NC1=O)C2=O ; *Oc1cccc2c1CN(C1CCC(=O)NC1=O)C2=O ; *N(C)c1cccc2c1C(=O)N(C1CCC(=O)NC1=O)C2=O ; *N(C)c1cccc2c1CN(C1CCC(=O)NC1=O)C2=O ; *C(=O)Nc1cccc2c1C(=O)N(C1CCC(=O)NC1=O)C2=O]

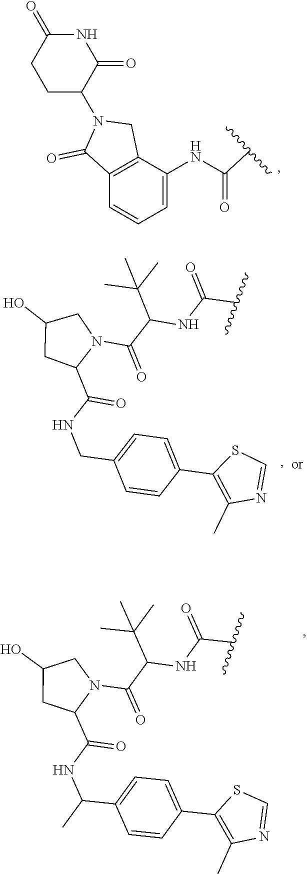

or a pharmaceutically acceptable salt thereof,
wherein “ [symbol] ” represents a point of attachment to the divalent linker (L).

In some embodiments, the ubiquitin ligase ligand (Q) is

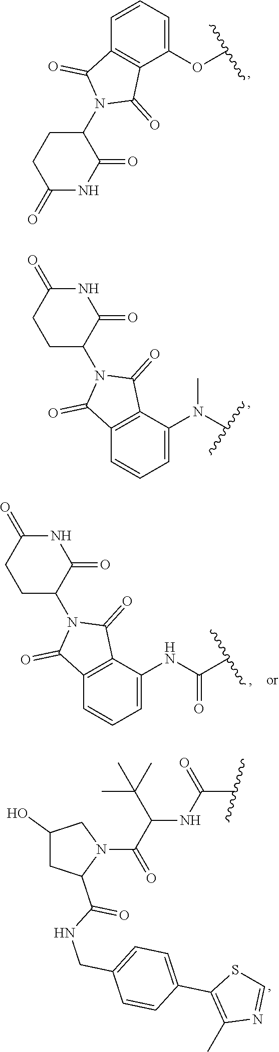

or a pharmaceutically acceptable salt thereof,
wherein “ [symbol] ” represents a point of attachment to the divalent linker (L).

In some embodiments, the ubiquitin ligase ligand (Q) is

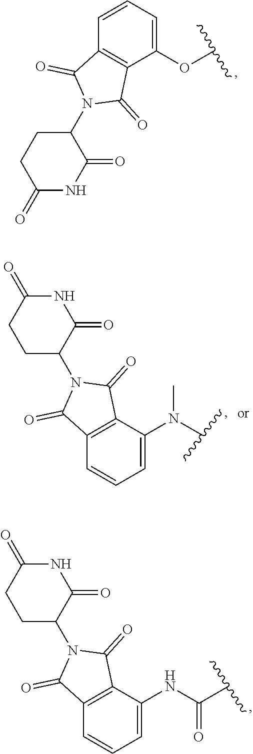 [SMILES: *Oc1cccc2c1C(=O)N(C1CCC(=O)NC1=O)C2=O ; *N(C)c1cccc2c1C(=O)N(C1CCC(=O)NC1=O)C2=O ; *C(=O)Nc1cccc2c1C(=O)N(C1CCC(=O)NC1=O)C2=O]

or a pharmaceutically acceptable salt thereof,
wherein “ [symbol] ” represents a point of attachment to the divalent linker (L).

In some embodiments, the radical of a protease ligand (e.g., a SARS-CoV-2 protease ligand) (P) is a target protein ligand. In some embodiments, the protease ligand (P) is of Formula I:

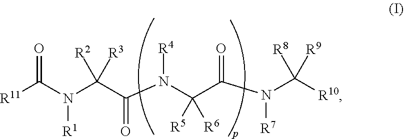

or a pharmaceutically acceptable salt thereof, wherein
each of R¹, R⁴, and R⁷ is independently H, C₁-C₆ alkyl, C₂-C₆ alkenyl, C₃-C₈ cycloalkyl, C₆-C₁₀ aryl, or 5- to 10-membered heteroaryl;
R² is C₁-C₆ alkylene-R^{A}, C₁-C₆ alkyl, C₂-C₆ alkenyl, C₃-C₈ cycloalkyl, 3- to 12-membered heterocycloalkyl, C₆-C₁₀ aryl, 5- to 10-membered heteroaryl, C₁-C₆ alkylene-C₆-C₁₀ aryl, or C₁-C₆alkylene-5- to 10-membered heteroaryl, wherein each hydrogen atom in C₁-C₆ alkyl, C₂-C₆ alkenyl, C₃-C₈ cycloalkyl, 3- to 12-membered heterocycloalkyl, C₆-C₁₀ aryl, or 5- to 10-membered heteroaryl is optionally substituted by R^{a};
each of R⁵ and R⁸ is independently C₁-C₆ alkyl, C₂-C₆ alkenyl, C₃-C₈ cycloalkyl, 3- to 12-membered heterocycloalkyl, C₆-C₁₀ aryl, 5- to 10-membered heteroaryl, C₁-C₆ alkylene-C₆-C₁₀ aryl, or C₁-C₆ alkylene-5- to 10-membered heteroaryl, wherein each hydrogen atom in C₁-C₆ alkyl, C₂-C₆ alkenyl, C₃-C₈ cycloalkyl, 3- to 12-membered heterocycloalkyl, C₆-C₁₀ aryl, or 5- to 10-membered heteroaryl is optionally substituted by R^{a}; or R⁴ and R⁵ together with the atoms to which they are attached combine to form a 5- to 8-membered heterocycloalkyl, wherein each hydrogen atom in 5- to 8-membered heterocycloalkyl is optionally substituted by R^{c};
each of R³, R⁶, and R⁹ is H;
R¹⁰ is C₁-C₆ alkyl-SO₃H, C₁-C₆ alkyl-CN, —C(O)H, —C(O)C₁-C₆ alkyl, —C(O)C(O)NR^{b}R^{b}, —C(O)COC(O)C₁-C₁₀ alkyl, —C(O)COC(O)C₆-C₁₀ aryl, —C(O)COC(O)-5- to 10-membered heteroaryl, or —CN, wherein each hydrogen atom in C₁-C₆ alkyl, and C₆-C₁₀ aryl is optionally substituted by halo, —OSO₃H, hydroxy, OC₁—C₆ alkyl, —CN, —NO₂, C₆-C₁₀ aryl, or —NR^{b}R^{b};
R¹¹ is R^{A}, —O—C₁-C₆ alkyl, —O—C₆-C₁₀ aryl, —O-5- to 10-membered heteroaryl, O—C₁-C₆ alkylene-C₆-C₁₀ aryl, or —O—C₁-C₆ alkylene-5- to 10-membered heteroaryl, wherein alkyl, alkylene, aryl, or heteroaryl is optionally substituted by R^{a};
R^{A} is

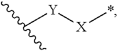

wherein [symbol] represents a point of attachment to the divalent linker (L), and * represents a point of attachment to P;
X is absent, NH, O, or S;
Y is a bond, C₁-C₆ alkylene, C₆-C₁₀ arylene, 5- to 10-membered heteroarylene, C₁-C₆ alkylene-C₆-C₁₀ arylene, or C₁-C₆ alkylene-5- to 10-membered heteroarylene;
each R^{a}, when present, is independently halo, hydroxy, C₁-C₆ alkyl, —O—C₁-C₆ alkyl, C₃-C₈ cycloalkyl, or 3- to 12-membered heterocycloalkyl;
each R^{b}, when present, is independently H, C₁-C₆ alkyl, C₂-C₆ alkenyl, C₃-C₈ cycloalkyl, or 3- to 12-membered heterocycloalkyl, wherein each hydrogen atom in C₁-C₆ alkyl, C₂-C₆ alkenyl, C₃-C₈ cycloalkyl, or 3- to 12-membered heterocycloalkyl is optionally substituted by R^{a};
each R^{c}, when present, is halo, hydroxy, C₁-C₆ alkyl, —O—C₁-C₆ alkyl, or C₃-C₈ cycloalkyl; or two R^{c} combine together with the atom or atoms to which they are attached to form a C₃-C₈ cycloalkyl, wherein each hydrogen atom in C₃-C₈ cycloalkyl is optionally substituted by R^{a}; and
p is 0 or 1;
provided that when R¹¹ is R^{A}, then R² is not alkylene-R^{A}.

In some embodiments, the protease ligand (P) is of Formula I′:

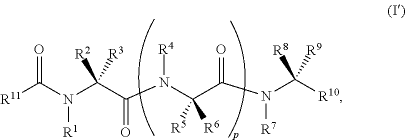

or a pharmaceutically acceptable salt thereof, wherein
each of R¹, R⁴, and R⁷ is independently H, C₁-C₆ alkyl, C₂-C₆ alkenyl, C₃-C₈ cycloalkyl, C₆-C₁₀ aryl, or 5- to 10-membered heteroaryl;
R² is C₁-C₆ alkylene-R^{A}, C₁-C₆ alkyl, C₂-C₆ alkenyl, C₃-C₈ cycloalkyl, 3- to 12-membered heterocycloalkyl, C₆-C₁₀ aryl, 5- to 10-membered heteroaryl, C₁-C₆ alkylene-C₆-C₁₀ aryl, or C₁-C₆alkylene-5- to 10-membered heteroaryl, wherein each hydrogen atom in C₁-C₆ alkyl, C₂-C₆ alkenyl, C₃-C₈ cycloalkyl, 3- to 12-membered heterocycloalkyl, C₆-C₁₀ aryl, or 5- to 10-membered heteroaryl is optionally substituted by R^{a};
each of R⁵ and R⁸ is independently C₁-C₆ alkyl, C₂-C₆ alkenyl, C₃-C₈ cycloalkyl, 3- to 12-membered heterocycloalkyl, C₆-C₁₀ aryl, 5- to 10-membered heteroaryl, C₁-C₆ alkylene-C₆-C₁₀ aryl, or C₁-C₆ alkylene-5- to 10-membered heteroaryl, wherein each hydrogen atom in C₁-C₆ alkyl, C₂-C₆ alkenyl, C₃-C₈ cycloalkyl, 3- to 12-membered heterocycloalkyl, C₆-C₁₀ aryl, or 5- to 10-membered heteroaryl is optionally substituted by R^{a}; or R⁴ and R⁵ together with the atoms to which they are attached combine to form a 5- to 8-membered heterocycloalkyl, wherein each hydrogen atom in 5- to 8-membered heterocycloalkyl is optionally substituted by R^{c};
each of R³, R⁶, and R⁹ is H;
R¹⁰ is C₁-C₆ alkyl-SO₃H, C₁-C₆ alkyl-CN, —C(O)H, —C(O)C₁-C₆ alkyl, —C(O)C(O)NR^{b}R^{b}, —C(O)COC(O)C₁-C₁₀ alkyl, —C(O)COC(O)C₆-C₁₀ aryl, —C(O)COC(O)-5- to 10-membered heteroaryl, or —CN, wherein each hydrogen atom in C₁-C₆ alkyl, and C₆-C₁₀ aryl is optionally substituted by halo, —OSO₃H, hydroxy, OC₁—C₆ alkyl, —CN, —NO₂, C₆-C₁₀ aryl, or —NR^{b}R^{b};
R¹¹ is R^{A}, —C₁-C₆ alkyl, —O—C₆-C₁₀ aryl, —O-5- to 10-membered heteroaryl, O—C₁-C₆ alkylene-C₆-C₁₀ aryl, or —O—C₁-C₆ alkylene-5- to 10-membered heteroaryl, wherein alkyl, alkylene, aryl, or heteroaryl is optionally substituted by R^{a};
R^{A} is

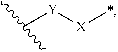

wherein [symbol] represents a point of attachment to the divalent linker (L), and * represents a point of attachment to P;
X is absent, NH, O, or S;
Y is a bond, C₁-C₆ alkylene, C₆-C₁₀ arylene, 5- to 10-membered heteroarylene, C₁-C₆ alkylene-C₆-C₁₀ arylene, or C₁-C₆ alkylene-5- to 10-membered heteroarylene;
each R^{a}, when present, is independently halo, hydroxy, C₁-C₆ alkyl, —O—C₁-C₆ alkyl, C₃-C₈ cycloalkyl, or 3- to 12-membered heterocycloalkyl;
each R^{b}, when present, is independently H, C₁-C₆ alkyl, C₂-C₆ alkenyl, C₃-C₈ cycloalkyl, or 3- to 12-membered heterocycloalkyl, wherein each hydrogen atom in C₁-C₆ alkyl, C₂-C₆ alkenyl, C₃-C₈ cycloalkyl, or 3- to 12-membered heterocycloalkyl is optionally substituted by R^{a};
each R^{c}, when present, is halo, hydroxy, C₁-C₆ alkyl, —O—C₁-C₆ alkyl, or C₃-C₈ cycloalkyl; or two R^{c} combine together with the atom or atoms to which they are attached to form a C₃-C₈ cycloalkyl, wherein each hydrogen atom in C₃-C₈ cycloalkyl is optionally substituted by R^{a}; and
p is 0 or 1;
provided that when R¹¹ is R^{A}, then R² is not alkylene-R^{A}.

In some embodiments, the protease ligand (P) is of Formula Ia:

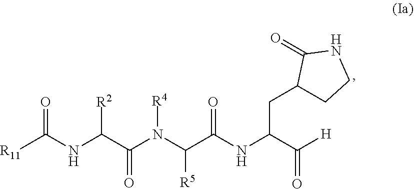

or a pharmaceutically acceptable salt thereof, wherein
R² is C₁-C₆ alkylene-R^{A}, C₁-C₆ alkyl, C₂-C₆ alkenyl, C₃-C₈ cycloalkyl, or 3- to 12-membered heterocycloalkyl, wherein each hydrogen atom in C₁-C₆ alkyl, C₂-C₆ alkenyl, C₃-C₈ cycloalkyl, or 3- to 12-membered heterocycloalkyl is optionally substituted by R^{a};
R⁴ is H;
R⁵ is C₁-C₆ alkyl, C₂-C₆ alkenyl, C₃-C₈ cycloalkyl, or 3- to 12-membered heterocycloalkyl, wherein each hydrogen atom in C₁-C₆ alkyl, C₂-C₆ alkenyl, C₃-C₈ cycloalkyl, or 3- to 12-membered heterocycloalkyl, is optionally substituted by R^{a}; or R⁴ and R⁵ together with the atoms to which they are attached combine to form a 5- to 8-membered heterocycloalkyl wherein each hydrogen atom in 5- to 8-membered heterocycloalkyl is optionally substituted by R^{c};
R¹¹ is R^{A}, —O—C₁—C₆ alkyl, —O—C₆-C₁₀ aryl, —O-5- to 10-membered heteroaryl, —O—C₁-C₆ alkylene-C₆-C₁₀ aryl, or —O—C₁-C₆ alkylene-5- to 10-membered heteroaryl, wherein alkyl, alkylene, aryl, or heteroaryl is optionally substituted by R^{a};
R^{A} is

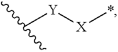

wherein [symbol] represents a point of attachment to the divalent linker (L), and * represents a point of attachment to P;
X is absent, NH, O, or S;
Y is a bond, C₁-C₆ alkylene, C₆-C₁₀ arylene, 5- to 10-membered heteroarylene, C₁-C₆ alkylene-C₆-C₁₀ arylene, or C₁-C₆ alkylene-5- to 10-membered heteroarylene;
each R^{a}, when present, is independently halo, hydroxy, C₁-C₆ alkyl, —O—C₁-C₆ alkyl, C₃-C₈ cycloalkyl, 3- to 12-membered heterocycloalkyl, C₆-C₁₀ aryl, or 5- to 10-membered heteroaryl; and
each R^{c}, when present, is halo, hydroxy, C₁-C₆ alkyl, OC₁—C₆ alkyl, or C₃-C₈ cycloalkyl; or two R^{c} combine together with the atom or atoms to which they are attached to form a C₃-C₈ cycloalkyl, wherein each hydrogen atom in C₃-C₈ cycloalkyl is optionally substituted by R^{a};
provided that when R¹¹ is R^{A}, then R² is not alkylene-R^{A}.

In some embodiments, the protease ligand (P) is of Formula Ia′:

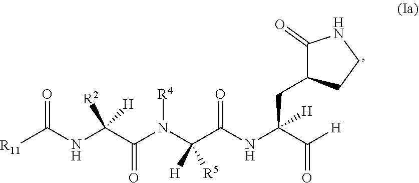

or a pharmaceutically acceptable salt thereof, wherein
R² is C₁-C₆ alkylene-R^{A}, C₁-C₆ alkyl, C₂-C₆ alkenyl, C₃-C₈ cycloalkyl, or 3- to 12-membered heterocycloalkyl, wherein each hydrogen atom in C₁-C₆ alkyl, C₂-C₆ alkenyl, C₃-C₈ cycloalkyl, or 3- to 12-membered heterocycloalkyl is optionally substituted by R^{a};
R⁴ is H;
R⁵ is C₁-C₆ alkyl, C₂-C₆ alkenyl, C₃-C₈ cycloalkyl, or 3- to 12-membered heterocycloalkyl, wherein each hydrogen atom in C₁-C₆ alkyl, C₂-C₆ alkenyl, C₃-C₈ cycloalkyl, or 3- to 12-membered heterocycloalkyl, is optionally substituted by R^{a}; or R⁴ and R⁵ together with the atoms to which they are attached combine to form a 5- to 8-membered heterocycloalkyl wherein each hydrogen atom in 5- to 8-membered heterocycloalkyl is optionally substituted by R^{c};
R¹¹ is R^{A}, —O—C₁—C₆ alkyl, —O—C₆-C₁₀ aryl, —O-5- to 10-membered heteroaryl, —O—C₁-C₆ alkylene-C₆-C₁₀ aryl, or —O—C₁-C₆ alkylene-5- to 10-membered heteroaryl, wherein alkyl, alkylene, aryl, or heteroaryl is optionally substituted by R^{a};
R^{A} is

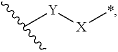

wherein [symbol] represents a point of attachment to the divalent linker (L), and * represents a point of attachment to P;
X is absent, NH, O, or S;
Y is a bond, C₁-C₆ alkylene, C₆-C₁₀ arylene, 5- to 10-membered heteroarylene, C₁-C₆ alkylene-C₆-C₁₀ arylene, or C₁-C₆ alkylene-5- to 10-membered heteroarylene;
each R^{a}, when present, is independently halo, hydroxy, C₁-C₆ alkyl, —O—C₁-C₆ alkyl, C₃-C₈ cycloalkyl, 3- to 12-membered heterocycloalkyl, C₆-C₁₀ aryl, or 5- to 10-membered heteroaryl;
each R^{c}, when present, is halo, hydroxy, C₁-C₆ alkyl, OC₁—C₆ alkyl, or C₃-C₈ cycloalkyl; or two R^{c} combine together with the atom or atoms to which they are attached to form a C₃-C₈ cycloalkyl, wherein each hydrogen atom in C₃-C₈ cycloalkyl is optionally substituted by R^{a};
n is 0, 1, or 2; and
m is 1, 2, 3, or 4;
provided that when R¹¹ is R^{A}, then R² is not alkylene-R^{A}.

In some embodiments, the protease ligand (P) is of Formula Ib:

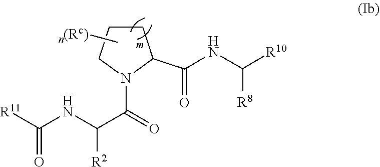

or a pharmaceutically acceptable salt thereof, wherein
R² is C₁-C₆ alkylene-R^{A}, C₁-C₆ alkyl, C₂-C₆ alkenyl, C₃-C₈ cycloalkyl, or 3- to 12-membered heterocycloalkyl, wherein each hydrogen atom in C₁-C₆ alkyl, C₂-C₆ alkenyl, C₃-C₈ cycloalkyl, or 3- to 12-membered heterocycloalkyl is optionally substituted by R^{a};
R⁸ is C₁-C₆ alkyl, C₂-C₆ alkenyl, C₃-C₈ cycloalkyl, 3- to 12-membered heterocycloalkyl, C₆-C₁₀ aryl, 5- to 10-membered heteroaryl, C₁-C₆ alkylene-C₆-C₁₀ aryl, or C₁-C₆ alkylene-5- to 10-membered heteroaryl, wherein each hydrogen atom in C₁-C₆ alkyl, C₂-C₆ alkenyl, C₃-C₈ cycloalkyl, 3- to 12-membered heterocycloalkyl, C₆-C₁₀ aryl, or 5- to 10-membered heteroaryl is optionally substituted by R^{a};
R¹⁰ is C₁-C₆ alkyl-SO₃H, C₁-C₆ alkyl-CN, —C(O)H, —C(O)C₁-C₆ alkyl, —C(O)C(O)NR^{b}R^{b}, —C(O)COC(O)C₁-C₁₀ alkyl, —C(O)COC(O)C₆-C₁₀ aryl, —C(O)COC(O)-5- to 10-membered heteroaryl, or —CN, wherein each hydrogen atom in C₁-C₆ alkyl, and C₆-C₁₀ aryl is optionally substituted by halo, —OSO₃H, hydroxy, OC₁—C₆ alkyl, —CN, —NO₂, C₆-C₁₀ aryl, or —NR^{b}R^{b};
R¹¹ is R^{A}, —O—C₁-C₆ alkyl, —O—C₆-C₁₀ aryl, —O-5- to 10-membered heteroaryl, —O—C₁-C₆ alkylene-C₆-C₁₀ aryl, or —O—C₁-C₆ alkylene-5- to 10-membered heteroaryl, wherein alkyl, alkylene, aryl, or heteroaryl is optionally substituted by R^{a};
R^{A} is

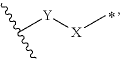

wherein [symbol] represents a point of attachment to the divalent linker (L), and * represents a point of attachment to P;
X is absent, NH, O, or S;
Y is a bond, C₁-C₆ alkylene, C₆-C₁₀ arylene, 5- to 10-membered heteroarylene, C₁-C₆ alkylene-C₆-C₁₀ arylene, or C₁-C₆ alkylene-5- to 10-membered heteroarylene;
each R^{a}, when present, is independently halo, hydroxy, C₁-C₆ alkyl, —O—C₁-C₆ alkyl, C₃-C₈ cycloalkyl, 3- to 12-membered heterocycloalkyl, C₆-C₁₀ aryl, or 5- to 10-membered heteroaryl; and
each R^{c}, when present, is halo, hydroxy, C₁-C₆ alkyl, OC₁—C₆ alkyl, or C₃-C₈ cycloalkyl; or two R^{c} combine together with the atom or atoms to which they are attached to form a C₃-C₈ cycloalkyl, wherein each hydrogen atom in C₃-C₈ cycloalkyl is optionally substituted by R^{a};
provided that when R¹¹ is R^{A}, then R² is not alkylene-R^{A}.

In some embodiments, the protease ligand (P) is of Formula Ib′:

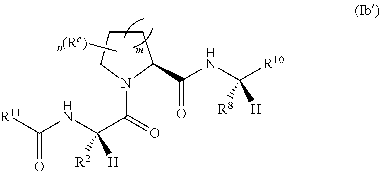

or a pharmaceutically acceptable salt thereof, wherein
R² is C₁-C₆ alkylene-R^{A}, C₁-C₆ alkyl, C₂-C₆ alkenyl, C₃-C₈ cycloalkyl, or 3- to 12-membered heterocycloalkyl, wherein each hydrogen atom in C₁-C₆ alkyl, C₂-C₆ alkenyl, C₃-C₈ cycloalkyl, or 3- to 12-membered heterocycloalkyl is optionally substituted by R^{a};
R⁸ is C₁-C₆ alkyl, C₂-C₆ alkenyl, C₃-C₈ cycloalkyl, 3- to 12-membered heterocycloalkyl, C₆-C₁₀ aryl, 5- to 10-membered heteroaryl, C₁-C₆ alkylene-C₆-C₁₀ aryl, or C₁-C₆ alkylene-5- to 10-membered heteroaryl, wherein each hydrogen atom in C₁-C₆ alkyl, C₂-C₆ alkenyl, C₃-C₈ cycloalkyl, 3- to 12-membered heterocycloalkyl, C₆-C₁₀ aryl, or 5- to 10-membered heteroaryl is optionally substituted by R^{a};
R¹⁰ is C₁-C₆ alkyl-SO₃H, C₁-C₆ alkyl-CN, —C(O)H, —C(O)C₁-C₆ alkyl, —C(O)C(O)NR^{b}R^{b}, —C(O)COC(O)C₁-C₁₀ alkyl, —C(O)COC(O)C₆-C₁₀ aryl, —C(O)COC(O)-5- to 10-membered heteroaryl, or —CN, wherein each hydrogen atom in C₁-C₆ alkyl, and C₆-C₁₀ aryl is optionally substituted by halo, —OSO₃H, hydroxy, OC₁—C₆ alkyl, —CN, —NO₂, C₆-C₁₀ aryl, or —NR^{b}R^{b};
R¹¹ is R^{A}, —O—C₁-C₆ alkyl, —O—C₆-C₁₀ aryl, —O-5- to 10-membered heteroaryl, —O—C₁-C₆ alkylene-C₆-C₁₀ aryl, or —O—C₁-C₆ alkylene-5- to 10-membered heteroaryl, wherein alkyl, alkylene, aryl, or heteroaryl is optionally substituted by R^{a};
R^{A} is

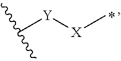

wherein [symbol] represents a point of attachment to the divalent linker (L), and * represents a point of attachment to P;
X is absent, NH, O, or S;
Y is a bond, C₁-C₆ alkylene, C₆-C₁₀ arylene, 5- to 10-membered heteroarylene, C₁-C₆ alkylene-C₆-C₁₀ arylene, or C₁-C₆ alkylene-5- to 10-membered heteroarylene;
each R^{a}, when present, is independently halo, hydroxy, C₁-C₆ alkyl, —O—C₁-C₆ alkyl, C₃-C₅ cycloalkyl, 3- to 12-membered heterocycloalkyl, C₆-C₁₀ aryl, or 5- to 10-membered heteroaryl;
each R^{c}, when present, is halo, hydroxy, C₁-C₆ alkyl, OC₁—C₆ alkyl, or C₃-C₈ cycloalkyl; or two R^{c} combine together with the atom or atoms to which they are attached to form a C₃-C₈ cycloalkyl, wherein each hydrogen atom in C₃-C₈ cycloalkyl is optionally substituted by R^{a};
n is 0, 1, or 2; and
m is 1, 2, 3, or 4;
provided that when R¹¹ is R^{A}, then R² is not alkylene-R^{A}.

In some embodiments, each of R¹, R⁴, and R⁷ is independently H, C₁-C₆ alkyl, C₂-C₆ alkenyl, C₃-C₈ cycloalkyl, C₆-C₁₀ aryl, or 5- to 10-membered heteroaryl. In some embodiments, each of R¹, R⁴, and R⁷ is independently H, C₁-C₆ alkyl, C₂-C₆ alkenyl, or C₃-C₈ cycloalkyl. In some embodiments, each of R¹, R⁴, and R⁷ is independently H or C₁-C₆ alkyl. In some embodiments, each of R¹, R⁴, and R⁷ is H. In some embodiments, each of R¹ and R⁷ is independently H.

In some embodiments, R¹ is H, C₁-C₆ alkyl, C₂-C₆ alkenyl, C₃-C₈ cycloalkyl, C₆-C₁₀ aryl, or 5- to 10-membered heteroaryl. In some embodiments, R¹ is H, C₁-C₆ alkyl, C₂-C₆ alkenyl, or C₃-C₈ cycloalkyl. In some embodiments, R¹ is H or C₁-C₆ alkyl. In some embodiments, R¹ is H.

In some embodiments, R² is alkylene-R^{A} (e.g., C₁-C₆ alkylene-R^{A}), (e.g., C₁-C₆ alkyl), alkenyl (e.g., C₂-C₆ alkenyl), cycloalkyl (e.g., C₃-C₅ cycloalkyl), heterocycloalkyl (e.g., 3- to 12-membered heterocycloalkyl), alkylene-aryl (e.g., C₁-C₆ alkylene-C₆-C₁₀ aryl), alkylene-heteroaryl (e.g., C₁-C₆ alkylene-5- to 10-membered heteroaryl), aryl (e.g., C₆-C₁₀ aryl), heteroaryl (e.g., 5- to 10-membered heteroaryl), alkylene-aryl (e.g., C₁-C₆ alkylene-C₆-C₁₀ aryl), or alkylene-heteroaryl (e.g., C₁-C₆ alkylene-5- to 10-membered heteroaryl), wherein each hydrogen atom in alkyl (e.g., C₁-C₆ alkyl), alkenyl (e.g., C₂-C₆ alkenyl), cycloalkyl (e.g., C₃-C₈ cycloalkyl), heterocycloalkyl (e.g., 3- to 12-membered heterocycloalkyl), alkylene-aryl (e.g., C₁-C₆ alkylene-C₆-C₁₀ aryl), alkylene-heteroaryl (e.g., C₁-C₆ alkylene-5- to 10-membered heteroaryl), aryl (e.g., C₆-C₁₀ aryl), heteroaryl (e.g., 5- to 10-membered heteroaryl), alkylene-aryl (e.g., C₁-C₆ alkylene-C₆-C₁₀ aryl), or alkylene-heteroaryl (e.g., C₁-C₆ alkylene-5- to 10-membered heteroaryl) is optionally substituted by R^{a}. In some embodiments, R² is alkylene-R^{A} (e.g., C₁-C₆ alkylene-R^{A}), alkyl (e.g., C₁-C₆ alkyl), alkenyl (e.g., C₂-C₆ alkenyl), cycloalkyl (e.g., C₃-C₈ cycloalkyl), heterocycloalkyl (e.g., 3- to 12-membered heterocycloalkyl), alkylene-aryl (e.g., C₁-C₆ alkylene-C₆-C₁₀ aryl), alkylene-heteroaryl (e.g., C₁-C₆ alkylene-5- to 10-membered heteroaryl), wherein each hydrogen atom in alkyl (e.g., C₁-C₆ alkyl), alkenyl (e.g., C₂-C₆ alkenyl), cycloalkyl (e.g., C₃-C₈ cycloalkyl), heterocycloalkyl (e.g., 3- to 12-membered heterocycloalkyl), aryl (e.g., C₆-C₁₀ aryl), or heteroaryl (e.g., 5- to 10-membered heteroaryl) is optionally substituted by R^{a}. In some embodiments, R² is C₁-C₆ alkylene-R^{A}, C₁-C₆ alkyl, C₂-C₆ alkenyl, C₃-C₈ cycloalkyl, or 3- to 12-membered heterocycloalkyl, wherein each hydrogen atom in C₁-C₆ alkyl, C₂-C₆ alkenyl, C₃-C₈ cycloalkyl, 3- to 12-membered heterocycloalkyl, is optionally substituted by R^{a}. In some embodiments, R² is C₁-C₆ alkyl, C₂-C₆ alkenyl, C₃-C₈ cycloalkyl, or 3- to 12-membered heterocycloalkyl, wherein each hydrogen atom in C₁-C₆ alkyl, C₂-C₆ alkenyl, C₃-C₈ cycloalkyl, or 3- to 12-membered heterocycloalkyl is optionally substituted by R^{a}.

In some embodiments, R² is optionally substituted C₁-C₆ alkyl (e.g., methyl, ethyl, n-propyl, iso-propyl, n-butyl, iso-butyl, tert-butyl). In some embodiments, R² is optionally substituted C₆-C₁₀ aryl. In some embodiments, R² is C₁-C₆ alkyl optionally substituted with C₁-C₆ alkyl, —O—C₁-C₆ alkyl, C₃-C₈ cycloalkyl, 3- to 12-membered heterocycloalkyl, C₆-C₁₀ aryl, or 5- to 10-membered heteroaryl. In some embodiments, R² is C₁-C₆ alkyl optionally substituted with C₁-C₆alkyl, —O—C₁-C₆ alkyl, C₃-C₈ cycloalkyl, or C₆-C₁₀ aryl. In some embodiments, R² is

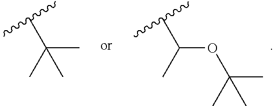

In some embodiments, R³ is H.

In some embodiments, R⁴ is H, C₁-C₆ alkyl, C₂-C₆ alkenyl, C₃-C₈ cycloalkyl, C₆-C₁₀ aryl, or 5- to 10-membered heteroaryl. In some embodiments, R⁴ is independently H, C₁-C₆ alkyl, C₂-C₆ alkenyl, or C₃-C₈ cycloalkyl. In some embodiments, R⁴ is independently H or C₁-C₆ alkyl.

In some embodiments, R⁵ is C₁-C₆ alkyl, C₂-C₆ alkenyl, C₃-C₈ cycloalkyl, 3- to 12-membered heterocycloalkyl, C₆-C₁₀ aryl, 5- to 10-membered heteroaryl, C₁-C₆ alkylene-C₆-C₁₀ aryl, or C₁-C₆ alkylene-5- to 10-membered heteroaryl, wherein each hydrogen atom in C₁-C₆ alkyl, C₂-C₆ alkenyl, C₃-C₈ cycloalkyl, 3- to 12-membered heterocycloalkyl, C₆-C₁₀ aryl, or 5- to 10-membered heteroaryl is optionally substituted by R^{a}; or R⁴ and R⁵ together with the atoms to which they are attached combine to form a 5- to 8-membered heterocycloalkyl, wherein each hydrogen atom in 5- to 8-membered heterocycloalkyl is optionally substituted by R^{c}. In some embodiments, R⁵ is C₁-C₆ alkyl, C₂-C₆ alkenyl, C₃-C₈ cycloalkyl, 3- to 12-membered heterocycloalkyl, C₆-C₁₀ aryl, 5- to 10-membered heteroaryl, C₁-C₆ alkylene-C₆-C₁₀ aryl, or C₁-C₆ alkylene-5- to 10-membered heteroaryl, wherein each hydrogen atom in C₁-C₆ alkyl, C₂-C₆ alkenyl, C₃-C₈ cycloalkyl, 3- to 12-membered heterocycloalkyl, C₆-C₁₀ aryl, or 5- to 10-membered heteroaryl is optionally substituted by R^{a}.

In some embodiments, R⁵ is optionally substituted C₁-C₆ alkyl (e.g., methyl, ethyl, n-propyl, iso-propyl, n-butyl, iso-butyl, tert-butyl). In some embodiments, R⁵ is optionally substituted C₁-C₆ alkylene-C₆-C₁₀ aryl or optionally substituted C₆-C₁₀ aryl. In some embodiments, R⁵ is C₁-C₆ alkyl optionally substituted with C₁-C₆ alkyl, —O—C₁-C₆ alkyl, C₃-C₈ cycloalkyl, 3- to 12-membered heterocycloalkyl, C₆-C₁₀ aryl, or 5- to 10-membered heteroaryl. In some embodiments, R⁵ is C₁-C₆ alkyl optionally substituted with C₁-C₆ alkyl, —O—C₁-C₆ alkyl, C₃-C₈ cycloalkyl, or C₆-C₁₀ aryl. In some embodiments, R⁵ is

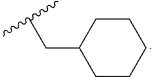 [SMILES: *CC1CCCCC1]

In some embodiments, R⁴ and R⁵ together with the atoms to which they are attached combine to form a 5- to 8-membered heterocycloalkyl, wherein each hydrogen atom in 5- to 8-membered heterocycloalkyl is optionally substituted by R^{c}.

In some embodiments, R⁴ and R⁵ together with the atoms to which they are attached combine to form

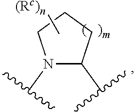

wherein n is 0, 1, or 2, and m is 1, 2, 3, or 4. In some embodiments, R⁴ and R⁵ together with the atoms to which they are attached combine to form

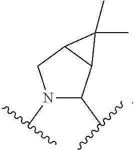 [SMILES: *C1C2C(CN1*)C2(C)C]

In some embodiments, R⁴ and R⁵ together with the atoms to which they are attached combine to form

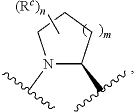

wherein n is 0, 1, or 2, and m is 1, 2, 3, or 4. In some embodiments, R⁴ and R⁵ together with the atoms to which they are attached combine to form

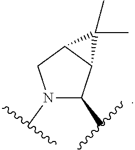 [SMILES: *[C@@H]1[C@@H]2[C@H](CN1*)C2(C)C]

In some embodiments, R⁶ is H.

In some embodiments, R⁷ is H, C₁-C₆ alkyl, C₂-C₆ alkenyl, C₃-C₈ cycloalkyl, C₆-C₁₀ aryl, or 5- to 10-membered heteroaryl. In some embodiments, R⁷ is H, C₁-C₆ alkyl, C₂-C₆ alkenyl, or C₃-C₈ cycloalkyl. In some embodiments, R⁷ is H or C₁-C₆ alkyl. In some embodiments, R¹ is H.

In some embodiments, R⁸ is C₁-C₆ alkyl, C₂-C₆ alkenyl, C₃-C₈ cycloalkyl, 3- to 12-membered heterocycloalkyl, C₆-C₁₀ aryl, 5- to 10-membered heteroaryl, C₁-C₆ alkylene-C₆-C₁₀ aryl, or C₁-C₆ alkylene-5- to 10-membered heteroaryl, wherein each hydrogen atom in C₁-C₆ alkyl, C₂-C₆ alkenyl, C₃-C₈ cycloalkyl, 3- to 12-membered heterocycloalkyl, C₆-C₁₀ aryl, or 5- to 10-membered heteroaryl is optionally substituted by R^{a}. In some embodiments, R⁸ is C₁-C₆ alkyl, C₂-C₆ alkenyl, C₆-C₁₀ aryl, or 5- to 10-membered heteroaryl, wherein each hydrogen atom in C₁-C₆ alkyl, C₂-C₆ alkenyl, C₆-C₁₀ aryl, or 5- to 10-membered heteroaryl is optionally substituted by R^{a}. In some embodiments, R⁸ is optionally substituted C₁-C₆ alkyl or optionally substituted 5- to 10-membered heteroaryl.

In some embodiments, R⁸ is optionally substituted C₁-C₆ alkyl (e.g., methyl, ethyl, n-propyl, iso-propyl, n-butyl, iso-butyl, tert-butyl). In some embodiments, R⁸ is optionally substituted 5- to 10-membered heteroaryl. In some embodiments, R⁸ is C₁-C₆ alkyl optionally substituted with 3- to 12-membered heterocycloalkyl. In some embodiments, R⁸ is 5- to 10-membered heteroaryl optionally substituted with halo. In some embodiments, R⁸ is

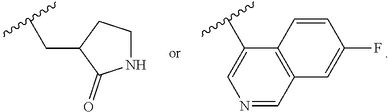 [SMILES: *CC1CCNC1=O ; *c1cncc2cc(F)ccc12]

In some embodiments, R⁹ is H.

In some embodiments, R¹⁰ is C₁-C₆ alkyl-SO₃H, C₁-C₆ alkyl-CN, —C(O)H, —C(O)C₁-C₆ alkyl, —C(O)C(O)NR^{b}R^{b}, —C(O)COC(O)C₁-C₁₀ alkyl, —C(O)COC(O)C₆-C₁₀ aryl, —C(O)COC(O)-5- to 10-membered heteroaryl, or —CN, wherein each hydrogen atom in C₁-C₆ alkyl, and C₆-C₁₀ aryl is optionally substituted by halo, —OSO₃H, hydroxy, —OC₁—C₆ alkyl, —CN, —NO₂, C₆-C₁₀ aryl, or —NR^{b}R^{b}. In some embodiments, R¹⁰ is C(O)H or —CN.

In some embodiments, R¹¹ is R^{A}, —O—C₁-C₆ alkyl, —O—C₆-C₁₀ aryl, —O-5- to 10-membered heteroaryl, —O—C₁-C₆ alkylene-C₆-C₁₀ aryl, or —O—C₁-C₆ alkylene-5- to 10-membered heteroaryl, wherein alkyl, alkylene, aryl, or heteroaryl is optionally substituted by R^{a}. In some embodiments, R¹¹ is R^{A}, —O—C₁-C₆ alkylene-C₆-C₁₀ aryl, or —O—C₁-C₆ alkylene-5- to 10-membered heteroaryl, wherein alkylene, aryl, or heteroaryl is optionally substituted by R^{a}. In some embodiments, R¹¹ is R^{A}. In some embodiments, R¹¹ is —O—C₁-C₆ alkylene-C₆-C₁₀ aryl. In some embodiments, R¹¹ is

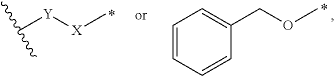

wherein [symbol] represents a point of attachment to the divalent linker (L), and * represents a point of attachment to P.

In some embodiments, R^{A} is

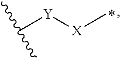

wherein [symbol] represents a point of attachment to the divalent linker (L), and * represents a point of attachment to P.

In some embodiments, X is absent, NH, O, or S. In some embodiments, X is NH, O, or S. In some embodiments, X is absent, NH, or O. In some embodiments, X is NH or O.

In some embodiments, Y is a bond, C₁-C₆ alkylene, C₆-C₁₀ arylene, 5- to 10-membered heteroarylene, C₁-C₆ alkylene-C₆-C₁₀ arylene, or C₁-C₆ alkylene-5- to 10-membered heteroarylene. In some embodiments, Y is a bond, C₁-C₆ alkylene, C₁-C₆ alkylene-C₆-C₁₀ arylene, or C₁-C₆ alkylene-5- to 10-membered heteroarylene. In some embodiments, Y is a bond, C₁-C₆ alkylene, or C₁-C₆ alkylene-C₆-C₁₀ arylene. In some embodiments, Y is selected from the group consisting of a bond,

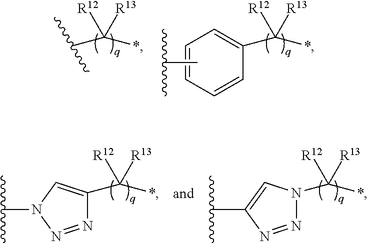

wherein each of R¹² and R¹³ is independently H, halo, hydroxy, C₁-C₆ alkyl, OC₁—C₆ alkyl), C₂-C₆ alkenyl, or C₃-C₈ cycloalkyl; and q is 1, 2, 3, 4, 5, or 6; and wherein “ [symbol] ” represents a point of attachment to the divalent linker (L), and * represents a point of attachment to P.

In some embodiments, Y is selected from the group consisting of a bond,

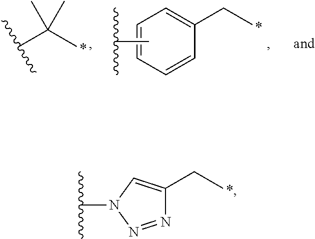

wherein “ [symbol] ” represents a point of attachment to the divalent linker (L), and * represents a point of attachment to P

In some embodiments, each R¹² is independently H, halo, hydroxy, C₁-C₆ alkyl, OC₁—C₆ alkyl), C₂-C₆ alkenyl, or C₃-C₈ cycloalkyl. In some embodiments, each R¹² is independently H or C₁-C₆ alkyl (e.g., methyl or ethyl). In some embodiments, each R¹² is independently H or methyl.

In some embodiments, each R¹³ is independently H, halo, hydroxy, C₁-C₆ alkyl, OC₁—C₆ alkyl), C₂-C₆ alkenyl, or C₃-C₈ cycloalkyl. In some embodiments, each R¹³ is independently H or C₁-C₆ alkyl (e.g., methyl or ethyl). In some embodiments, each R¹³ is independently H or methyl.

In some embodiments, each R^{a}, when present, is independently halo, hydroxy, C₁-C₆ alkyl, —O—C₁-C₆ alkyl, C₃-C₈ cycloalkyl, or 3- to 12-membered heterocycloalkyl.

In some embodiments, each R^{b}, when present, is independently H, C₁-C₆ alkyl, C₂-C₆ alkenyl, C₃-C₈ cycloalkyl, or 3- to 12-membered heterocycloalkyl, wherein each hydrogen atom in C₁-C₆ alkyl, C₂-C₆ alkenyl, C₃-C₈ cycloalkyl, or 3- to 12-membered heterocycloalkyl is optionally substituted by R^{a}.

In some embodiments, each R^{c}, when present, is halo, hydroxy, C₁-C₆ alkyl, —O—C₁-C₆ alkyl, or C₃-C₈ cycloalkyl; or two R^{c} combine together with the atom or atoms to which they are attached to form a C₃-C₈ cycloalkyl, wherein each hydrogen atom in C₃-C₈ cycloalkyl is optionally substituted by R^{a}. In some embodiments, two R^{c} combine together with the atom or atoms to which they are attached to form a C₃-C₈ cycloalkyl (e.g., cyclopropyl), wherein each hydrogen atom in C₃-C₈ cycloalkyl is optionally substituted by halo, hydroxy, or C₁-C₆ alkyl (e.g., methyl).

In some embodiments, p is 0 or 1. In some embodiments, p is 1.

In some embodiments, n is 0, 1, or 2. In some embodiments, n is 1 or 2. In some embodiments, n is 2.

In some embodiments, m is 1, 2, 3, or 4. In some embodiments, m is 1 or 2. In some embodiments, m is 1.

In some embodiments, q is 1, 2, 3, 4, 5, or 6. In some embodiments, q is 1, 2, 3, or 4. In some embodiments, q is 1 or 2. In some embodiments, q is 1.

In some embodiments, the protease ligand (P) is selected from the group consisting of

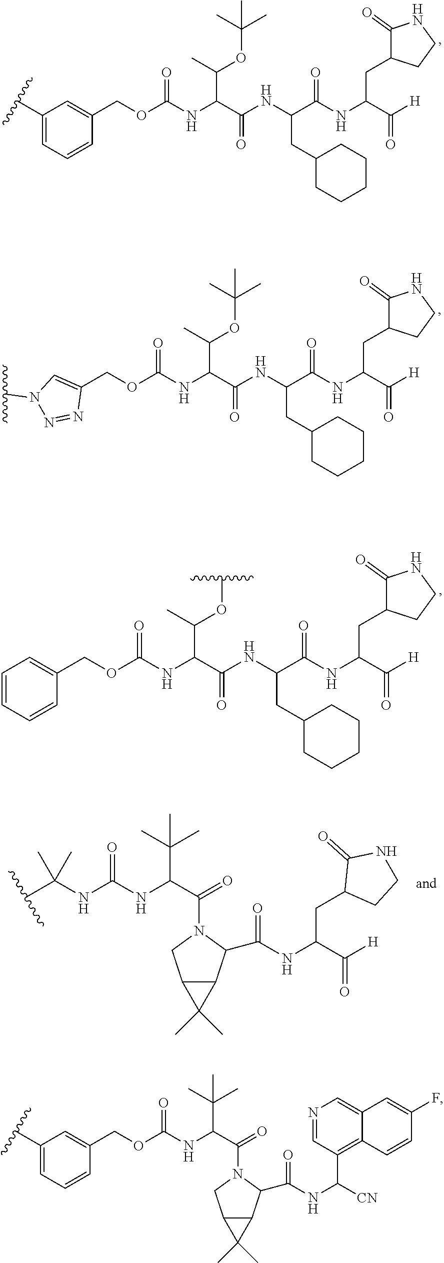

or a pharmaceutically acceptable salt thereof;
wherein “ [symbol] ” represents a point of attachment to the divalent linker (L).

In some embodiments, the protease ligand (P) is selected from the group consisting of

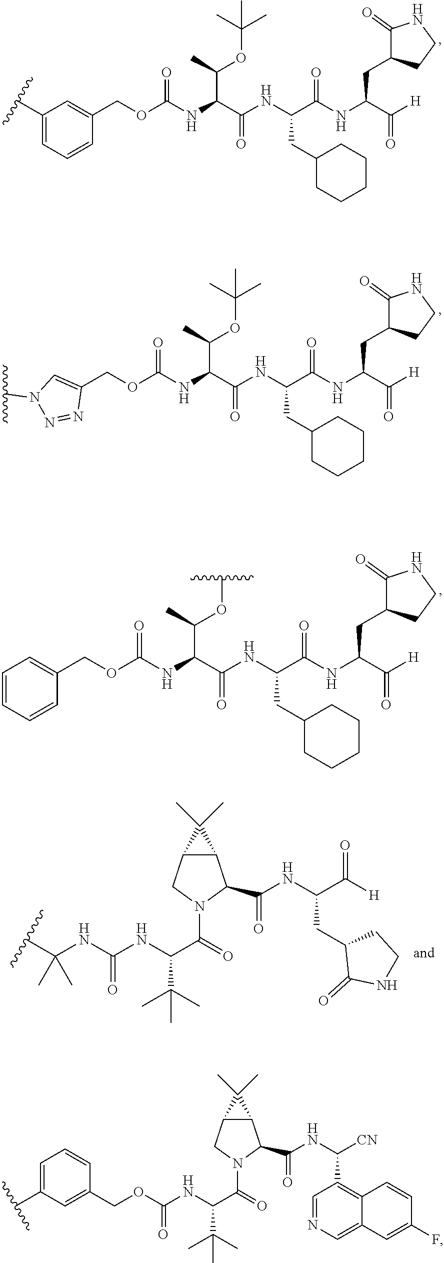 [SMILES: *c1cccc(COC(=O)N[C@H](C(=O)N[C@@H](CC2CCCCC2)C(=O)N[C@H](C=O)C[C@@H]2CCNC2=O)[C@@H](C)OC(C)(C)C)c1 ; *n1cc(COC(=O)N[C@H](C(=O)N[C@@H](CC2CCCCC2)C(=O)N[C@H](C=O)C[C@@H]2CCNC2=O)[C@@H](C)OC(C)(C)C)nn1 ; *O[C@H](C)[C@H](NC(=O)OCc1ccccc1)C(=O)N[C@@H](CC1CCCCC1)C(=O)N[C@H](C=O)C[C@@H]1CCNC1=O ; *C(C)(C)NC(=O)N[C@H](C(=O)N1C[C@H]2[C@@H]([C@H]1C(=O)N[C@H](C=O)C[C@@H]1CCNC1=O)C2(C)C)C(C)(C)C ; *c1cccc(COC(=O)N[C@H](C(=O)N2C[C@H]3[C@@H]([C@H]2C(=O)N[C@H](C#N)c2cncc4cc(F)ccc24)C3(C)C)C(C)(C)C)c1]

or a pharmaceutically acceptable salt thereof;
wherein “ [symbol] ” represents a point of attachment to the divalent linker (L).

It will be appreciated that linkers useful in connection with the present disclosure are not particularly limited by structure. The linker can be any linker of from about 2 to about 100 atoms in length and composed of elements including C, N, O and S that covalently attaches a ubiquitin ligase ligand (Q) to a protease ligand (P). In some embodiments, the linker can be any linker of from 4 to 50 atoms in length along the linker chain of atoms. In some embodiments, the linker can be any linker of from 4 to 25 atoms in length along the linker chain of atoms. It will be appreciated that the ranges provided herein for linker length in term of atom number in the chain can include any boundary numbers between and including 2 and 100, such as 5, 10, 15, 20, 25, 35, 45, 60, 75, 80, and 100. In some embodiments, the linker can be any linker of from 5 Å to about 100 Å in length. In some embodiments, the linker can be any linker of from 5 Å to about 50 Å in length. In some embodiments, the linker can be any linker of from 5 Å to about 40 Å in length. In some embodiments, the linker can be any linker of from 15 Å to about 25 Å in length. It will be appreciated that the ranges provided herein for linker length can include any boundary numbers between and including 5 and 100.

In some embodiments, the linker (L) can include one or more spacer linker portions. It will be appreciated that the structure of spacer linker portions included in the conjugates of the present disclosure are not particularly limited in structure. In some embodiments, the linker comprises simple groups, such as alkyl chain portions, ether portions (e.g. PEG), long chain amine portions, amino acid chain portions, and the like, and combinations thereof. In some embodiments, the linker (L) is selected from the group consisting of alkylene, alkenylene, alkynylene, arylene, heteroarylene, ether, amino, amide, carbamate, carbonate, ester, ketone, sulfate, sulfonamide, sulfoxide, sulfone, sulfonate, thioester, thioether, alkoxy, urea, hydrazine, and any combination thereof. In some embodiments, linkers useful in connection with the present disclosure comprise at least one portion selected from the group consisting of C₁-C₁₂ alkylene, C₁-C₁₂ alkylene-O—, C₁-C₁₂ alkylene-O—C₁-C₁₂ alkylene, C₁-C₁₂ alkylene-NH—, C₁-C₁₂ alkylene-N(C₁-C₆ alkyl)-, C₁-C₁₂ alkylene-NH—C₁-C₁₂ alkylene, C₁-C₁₂ alkylene-N(C₁-C₆ alkyl)-C₁-C₁₂ alkylene, C₁-C₁₂ alkylene-NH—C₁-C₁₂ alkylene-O—, C₁-C₁₂ alkylene-N(C₁-C₆ alkyl)-C₁-C₁₂ alkylene-O—, C₁-C₁₂ alkylene-NHC(O)—, C₁-C₁₂ alkylene-N(C₁-C₆ alkyl)C(O)—, C₁-C₁₂ alkylene-NHC(O)—C₁-C₁₂ alkylene, C₁-C₁₂ alkylene-N(C₁-C₆ alkyl)C(O)—C₁-C₁₂ alkylene, C₁-C₁₂ alkylene-NHC(O)—C₁-C₁₂ alkylene-O—, C₁-C₁₂ alkylene-N(C₁-C₆ alkyl)C(O)—C₁-C₁₂ alkylene-O—, —C(O)—C₁-C₁₂ alkyl-C(O)—, —NH—C₁-C₁₂ alkyl-NH—, —N(C₁-C₆ alkyl)-C₁-C₁₂ alkyl-N(C₁-C₆ alkyl)-, C₁-C₁₂ alkylene-NHC(O)—C₁-C₁₂ alkylene-(OCH₂CH₂)ₓ, C₁-C₁₂ alkylene-N(C₁-C₆ alkyl)C(O)—C₁-C₁₂ alkylene-(OCH₂CH₂)ₓ, C₁-C₁₂ alkylene-(OCH₂CH₂)ₓ, —C(O)CH₂CH₂(OCH₂CH₂)ₓ—, —C(O)CH₂CH₂(OCH₂CH₂)ₓNH—, and —C(O)CH₂CH₂(OCH₂CH₂)ₓN(C₁-C₆ alkyl)-; wherein x is an integer from 1 to 40. In some embodiments, the linker (L) is selected from the group consisting of C₁-C₁₂ alkylene, C₁-C₁₂ alkylene-O—, C₁-C₁₂ alkylene-O—C₁-C₁₂ alkylene, C₁-C₁₂ alkylene-N(C₁-C₆ alkyl)-C₁-C₁₂ alkylene, C₁-C₁₂ alkylene-N(C₁-C₆ alkyl)-C₁-C₁₂ alkylene-O—, C₁-C₁₂ alkylene-NHC(O)—C₁-C₁₂ alkylene, C₁-C₁₂ alkylene-N(C₁-C₆ alkyl)C(O)—C₁-C₁₂ alkylene, C₁-C₁₂ alkylene-N(C₁-C₆ alkyl)C(O)—C₁-C₁₂ alkylene-(OCH₂CH₂)ₓ, and C₁-C₁₂ alkylene-(OCH₂CH₂)ₓ; wherein x is an integer from 1 to 40. In some embodiments, the linker (L) is selected from the group consisting of C₂-C₁₀ alkylene, C₂-C₁₀ alkylene-O—, C₂-C₁₀ alkylene-O—C₂-C₁₀ alkylene, C₂-C₁₀ alkylene-N(C₁-C₆ alkyl)-C₂-C₁₀ alkylene, C₂-C₁₀ alkylene-N(CH₃)—C₂-C₁₀ alkylene-O—, C₁-C₁₂ alkylene-NHC(O)—C₂-C₁₀ alkylene, C₂-C₁₀ alkylene-N(CH₃)C(O)—C₂-C₁₀ alkylene, C₂-C₁₀ alkylene-N(CH₃)C(O)—C₂-C₁₀ alkylene-(OCH₂CH₂)ₓ, and C₂-C₁₀ alkylene-(OCH₂CH₂)ₓ; wherein x is an integer from 1 to 40.

In some embodiments, x is 2. In some embodiments, x is 3. In some embodiments, x is 4. In some embodiments, x is 5. It will be appreciated that the value of x is not particularly limited, and can be any value between 1 and about 40.

In some embodiments, the linker (L) is selected from the group consisting of

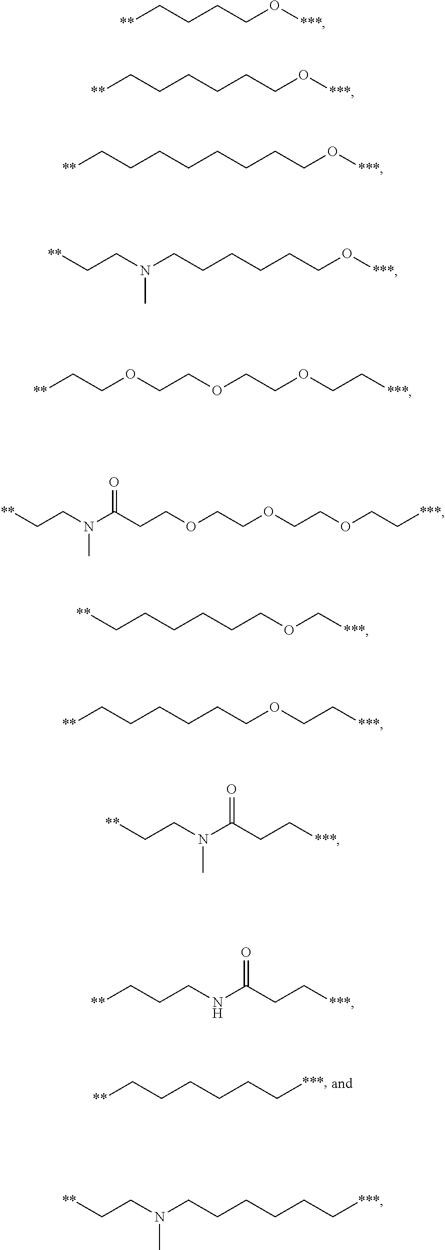

wherein ** represents a point of attachment to uniqitin ligase ligand (Q) and *** represents a point of attachment to protease ligand (P).

The following represent illustrative embodiments of compounds of the disclosure:

| # | Structure |
|---|---|
| 1 | 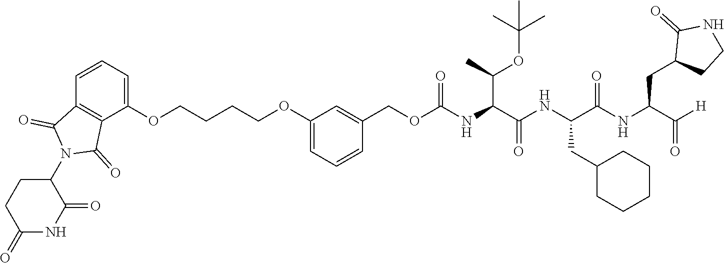 [SMILES: C[C@@H](OC(C)(C)C)[C@H](NC(=O)OCc1cccc(OCCCCOc2cccc3c2C(=O)N(C2CCC(=O)NC2=O)C3=O)c1)C(=O)N[C@@H](CC1CCCCC1)C(=O)N[C@H](C=O)C[C@@H]1CCNC1=O] |
| | 3-(4-((2-(2,6-dioxopiperidin-3-yl)-1,3-dioxoisoindolin-4-yl)oxy)butoxy)benzyl |
| | ((2S,3R)-3-(tert-butoxy)-1-(((S)-3-cyclohexyl-1-oxo-1-(((S)-1-oxo-3-((S)-2- |
| | oxopyrrolidin-3-yl)propan-2-yl)amino)propan-2-yl)amino)-1-oxobutan-2-yl)carbamate |
| | (MPD1) |
| 2 | 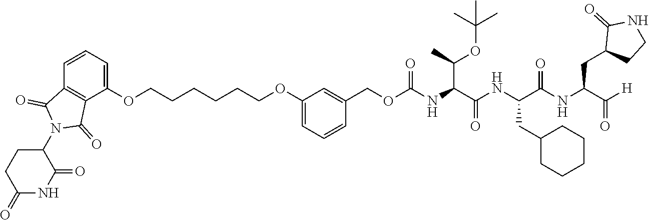 [SMILES: C[C@@H](OC(C)(C)C)[C@H](NC(=O)OCc1cccc(OCCCCCCOc2cccc3c2C(=O)N(C2CCC(=O)NC2=O)C3=O)c1)C(=O)N[C@@H](CC1CCCCC1)C(=O)N[C@H](C=O)C[C@@H]1CCNC1=O] |
| | 3-((6-((2-(2,6-dioxopiperidin-3-yl)-1,3-dioxoisoindolin-4-yl)oxy)hexyl)oxy)benzyl |
| | ((2S,3R)-3-(tert-butoxy)-1-(((S)-3-cyclohexyl-1-oxo-1-(((S)-1-oxo-3-((S)-2- |
| | oxopyrrolidin-3-yl)propan-2-yl)amino)propan-2-yl)amino)-1-oxobutan-2-yl)carbamate |
| | (MPD2) |
| 3 | 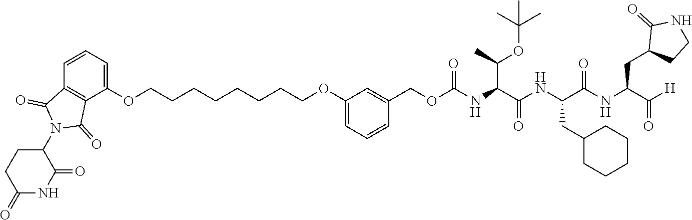 [SMILES: C[C@@H](OC(C)(C)C)[C@H](NC(=O)OCc1cccc(OCCCCCCCCOc2cccc3c2C(=O)N(C2CCC(=O)NC2=O)C3=O)c1)C(=O)N[C@@H](CC1CCCCC1)C(=O)N[C@H](C=O)C[C@@H]1CCNC1=O] |
| | 3-((8-((2-(2,6-dioxopiperidin-3-yl)-1,3-dioxoisoindolin-4-yl)oxy)octyl)oxy)benzyl |
| | ((2S,3R)-3-(tert-butoxy)-1-(((S)-3-cyclohexyl-1-oxo-1-(((S)-1-oxo-3-((S)-2- |
| | oxopyrrolidin-3-yl)propan-2-yl)amino)propan-2-yl)amino)-1-oxobutan-2-yl)carbamate |
| | (MPD3) |
| 4 | 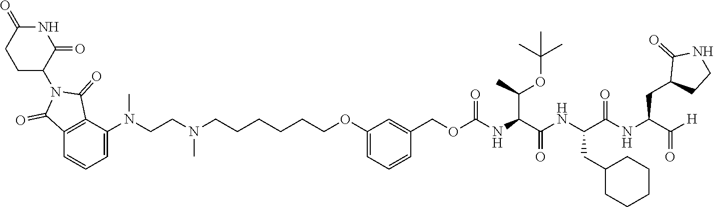 [SMILES: C[C@@H](OC(C)(C)C)[C@H](NC(=O)OCc1cccc(OCCCCCCN(C)CCN(C)c2cccc3c2C(=O)N(C2CCC(=O)NC2=O)C3=O)c1)C(=O)N[C@@H](CC1CCCCC1)C(=O)N[C@H](C=O)C[C@@H]1CCNC1=O] |
| | 3-((6-((2-((2-(2,6-dioxopiperidin-3-yl)-1,3-dioxoisoindolin-4- |
| | yl)(methyl)amino)ethyl)(methyl)amino)hexyl)oxy)benzyl ((2S,3R)-3-(tert-butoxy)-1- |
| | (((S)-3-cyclohexyl-1-oxo-1-(((S)-1-oxo-3-((S)-2-oxopyrrolidin-3-yl)propan-2- |
| | yl)amino)propan-2-yl)amino)-1-oxobutan-2-yl)carbamate (MPD4) |
| 5 | 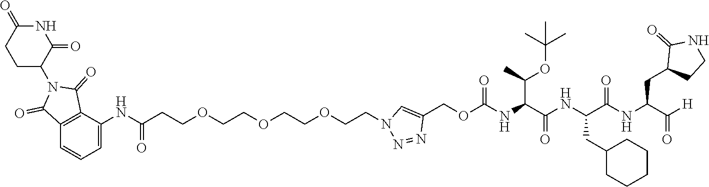 [SMILES: C[C@@H](OC(C)(C)C)[C@H](NC(=O)OCc1cn(CCOCCOCCOCCC(=O)Nc2cccc3c2C(=O)N(C2CCC(=O)NC2=O)C3=O)nn1)C(=O)N[C@@H](CC1CCCCC1)C(=O)N[C@H](C=O)C[C@@H]1CCNC1=O] |
| | (1-(2-(2-(2-(3-((2-(2,6-dioxopiperidin-3-yl)-1,3-dioxoisoindolin-4-yl)amino)-3- |
| | oxopropoxy)ethoxy)ethoxy)ethyl)-1H-1,2,3-triazol-4-yl)methyl ((2S,3R)-3-(tert- |
| | butoxy)-1-(((S)-3-cyclohexyl-1-oxo-1-(((S)-1-oxo-3-((S)-2-oxopyrrolidin-3-yl)propan- |
| | 2-yl)amino)propan-2-yl)amino)-1-oxobutan-2-yl)carbamate (MPD5) |
| 6 | 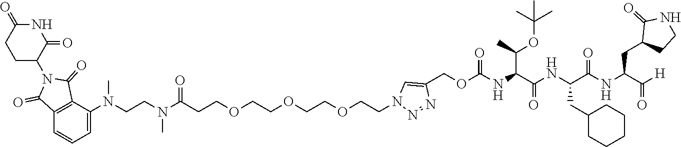 [SMILES: C[C@@H](OC(C)(C)C)[C@H](NC(=O)OCc1cn(CCOCCOCCOCCC(=O)N(C)CCN(C)c2cccc3c2C(=O)N(C2CCC(=O)NC2=O)C3=O)nn1)C(=O)N[C@@H](CC1CCCCC1)C(=O)N[C@H](C=O)C[C@@H]1CCNC1=O] |
| | (1-(2-(2-(2,6-dioxopiperidin-3-yl)-1,3-dioxoisoindolin-4-yl)-5-methyl-6-oxo-9,12,15- |
| | trioxa-2,5-diazaheptadecan-17-yl)-1H-1,2,3-triazol-4-yl)methyl ((2S,3R)-3-(tert- |
| | butoxy)-1-(((S)-3-cyclohexyl-1-oxo-1-(((S)-1-oxo-3-((S)-2-oxopyrrolidin-3-yl)propan- |
| | 2-yl)amino)propan-2-yl)amino)-1-oxobutan-2-yl)carbamate (MPD6) |
| 7 | 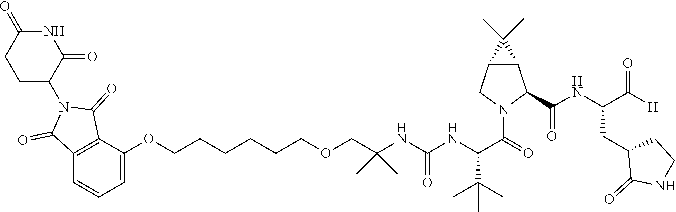 [SMILES: CC(C)(COCCCCCCOc1cccc2c1C(=O)N(C1CCC(=O)NC1=O)C2=O)NC(=O)N[C@H](C(=O)N1C[C@H]2[C@@H]([C@H]1C(=O)N[C@H](C=O)C[C@@H]1CCNC1=O)C2(C)C)C(C)(C)C] |
| | (1R,2S,5S)-3-((2S)-2-(3-(1-((6-((2-(2,6-dioxopiperidin-3-yl)-1,3-dioxoisoindolin-4- |
| | yl)oxy)hexyl)oxy)-2-methylpropan-2-yl)ureido)-3,3-dimethylbutanoyl)-6,6-dimethyl- |
| | N-((S)-1-oxo-3-((S)-2-oxopyrrolidin-3-yl)propan-2-yl)-3-azabicyclo[3.1.0]hexane-2- |
| | carboxamide (MPD7) |
| 8 | 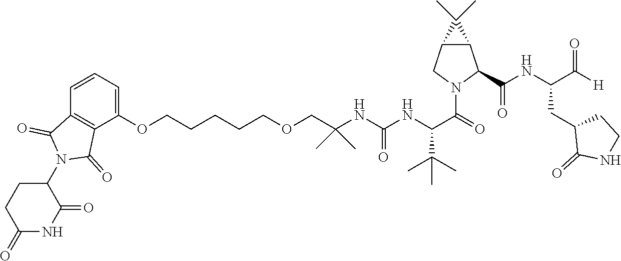 [SMILES: CC(C)(COCCCCCOc1cccc2c1C(=O)N(C1CCC(=O)NC1=O)C2=O)NC(=O)N[C@H](C(=O)N1C[C@H]2[C@@H]([C@H]1C(=O)N[C@H](C=O)C[C@@H]1CCNC1=O)C2(C)C)C(C)(C)C] |
| | (1R,2S,5S)-3-((2S)-2-(3-(4-((6-((2-(2,6-dioxopiperidin-3-yl)-1,3-dioxoisoindolin-4- |
| | yl)oxy)hexyl)oxy)-2-methylbutan-2-yl)ureido)-3,3-dimethylbutanoyl)-6,6-dimethyl-N- |
| | ((S)-1-oxo-3-((S)-2-oxopyrrolidin-3-yl)propan-2-yl)-3-azabicyclo[3.1.0]hexane-2- |
| | carboxamide (MPD8) |
| 9 | 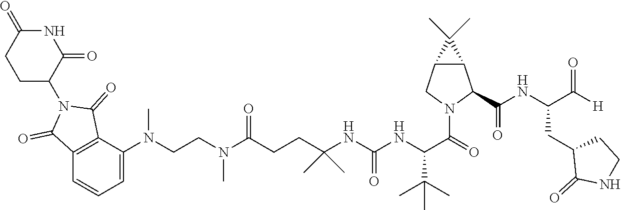 [SMILES: CN(CCN(C)c1cccc2c1C(=O)N(C1CCC(=O)NC1=O)C2=O)C(=O)CCC(C)(C)NC(=O)N[C@H](C(=O)N1C[C@H]2[C@@H]([C@H]1C(=O)N[C@H](C=O)C[C@@H]1CCNC1=O)C2(C)C)C(C)(C)C] |
| | (1R,2S,5S)-3-((13S)-13-(tert-butyl)-2-(2-(2,6-dioxopiperidin-3-yl)-1,3- |
| | dioxoisoindolin-4-yl)-5,9,9-trimethyl-6,11-dioxo-2,5,10,12-tetraazatetradecan-14-oyl)- |
| | 6,6-dimethyl-N-((S)-1-oxo-3-((S)-2-oxopyrrolidin-3-yl)propan-2-yl)-3- |
| | azabicyclo[3.1.0]hexane-2-carboxamide (MPD9) |
| 10 | 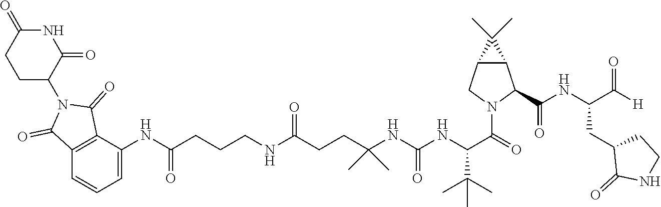 [SMILES: CC(C)(CCC(=O)NCCCC(=O)Nc1cccc2c1C(=O)N(C1CCC(=O)NC1=O)C2=O)NC(=O)N[C@H](C(=O)N1C[C@H]2[C@@H]([C@H]1C(=O)N[C@H](C=O)C[C@@H]1CCNC1=O)C2(C)C)C(C)(C)C] |
| | (1R,2S,5S)-3-((2S)-2-(3-(5-((4-((2-(2,6-dioxopiperidin-3-yl)-1,3-dioxoisoindolin-4- |
| | yl)amino)-4-oxobutyl)amino)-2-methyl-5-oxopentan-2-yl)ureido)-3,3- |
| | dimethylbutanoyl)-6,6-dimethyl-N-((S)-1-oxo-3-((S)-2-oxopyrrolidin-3-yl)propan-2- |
| | yl)-3-azabicyclo[3.1.0]hexane-2-carboxamide (MPD10) |
| 11 | 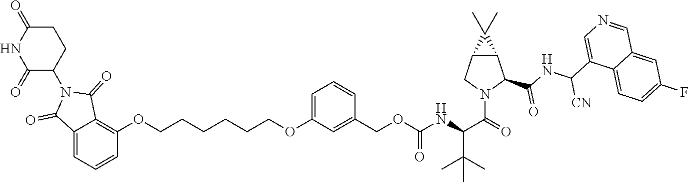 [SMILES: CC(C)(C)[C@@H](NC(=O)OCc1cccc(OCCCCCCOc2cccc3c2C(=O)N(C2CCC(=O)NC2=O)C3=O)c1)C(=O)N1C[C@H]2[C@@H]([C@H]1C(=O)NC(C#N)c1cncc3cc(F)ccc13)C2(C)C] |
| | 3-((6-((2-(2,6-Dioxopiperidin-3-yl)-1,3-dioxoisoindolin-4-yl)oxy)hexyl)oxy)benzyl |
| | ((2S)-1-((1R,2S,5S)-2-((cyano(7-fluoroisoquinolin-4-yl)methyl)carbamoyl)-6,6- |
| | dimethyl-3-azabicyclo[3.1.0]hexan-3-yl)-3,3-dimethyl-1-oxobutan-2-yl)carbamate |
| | (MPD11) |
| 12 | 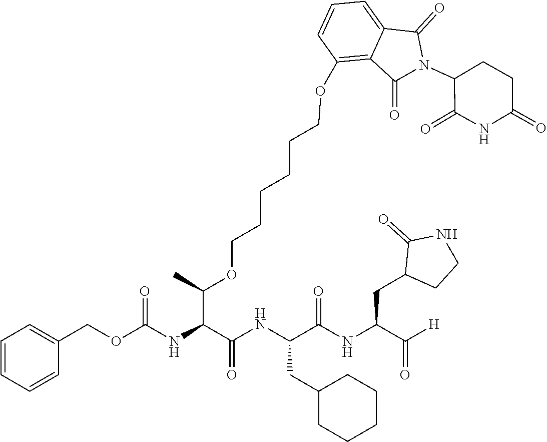 [SMILES: C[C@@H](OCCCCCCOc1cccc2c1C(=O)N(C1CCC(=O)NC1=O)C2=O)[C@H](NC(=O)OCc1ccccc1)C(=O)N[C@@H](CC1CCCCC1)C(=O)N[C@H](C=O)CC1CCNC1=O] |
| 13 | 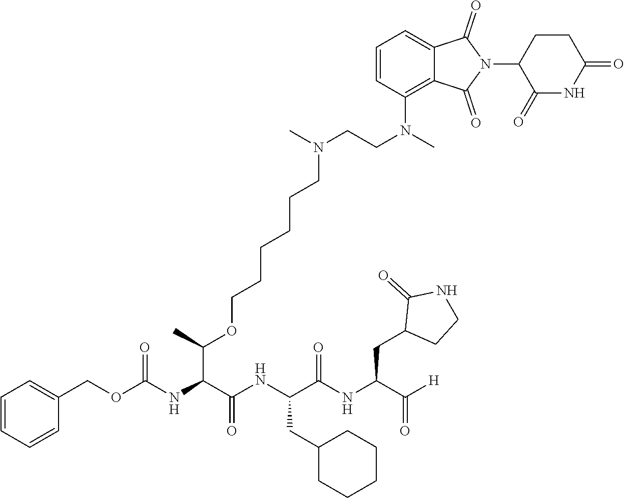 [SMILES: C[C@@H](OCCCCCCN(C)CCN(C)c1cccc2c1C(=O)N(C1CCC(=O)NC1=O)C2=O)[C@H](NC(=O)OCc1ccccc1)C(=O)N[C@@H](CC1CCCCC1)C(=O)N[C@H](C=O)CC1CCNC1=O] |

Those skilled in the art will recognize that the species listed or illustrated herein are not exhaustive, and that additional species within the scope of these defined terms may also be selected.

### Pharmaceutical Compositions

For treatment purposes, pharmaceutical compositions comprising the compounds described herein may further comprise one or more pharmaceutically-acceptable excipients. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the invention are sterile compositions. Pharmaceutical compositions may be prepared using compounding techniques known or that become available to those skilled in the art.

Sterile compositions are also contemplated by the invention, including compositions that are in accord with national and local regulations governing such compositions.

The pharmaceutical compositions and compounds described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the invention may be administered by a suitable route of delivery, such as oral, parenteral, rectal, nasal, topical, or ocular routes, or by inhalation. Preferably, the compositions are formulated for intravenous or oral administration.

For oral administration, the compounds the invention may be provided in a solid form, such as a tablet or capsule, or as a solution, emulsion, or suspension. To prepare the oral compositions, the compounds of the invention may be formulated to yield a dosage of, e.g., from about 0.1 mg to 1 g daily, or about 1 mg to 50 mg daily, or about 50 to 250 mg daily, or about 250 mg to 1 g daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil, such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

For parenteral use, including intravenous, intramuscular, intraperitoneal, intranasal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For nasal, inhaled, or oral administration, the inventive pharmaceutical compositions may be administered using, for example, a spray formulation also containing a suitable carrier. The inventive compositions may be formulated for rectal administration as a suppository.

For topical applications, the compounds of the present invention are preferably formulated as creams or ointments or a similar vehicle suitable for topical administration. For topical administration, the inventive compounds may be mixed with a pharmaceutical carrier at a concentration of about 0.1% to about 10% of drug to vehicle. Another mode of administering the agents of the invention may utilize a patch formulation to effect transdermal delivery.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

The term “subject” refers to a mammalian patient in need of such treatment, such as a human.

Exemplary diseases include those caused by SARS-CoV-2, SARS-CoV, MERS-CoV, Ebola virus, Paramyxoviruses, Bunyaviruses (Bunyavirales), Togaviruses, Filoviruses, Picornaviruses, Flaviviruses. In some examples, the disease is caused by SARS-CoV-2.

In some embodiments, the disclosure provides a pharmaceutical composition comprising at least one compound of the present disclosure, or a pharmaceutically acceptable salt thereof, and optionally one or more pharmaceutically acceptable excipients.

In some embodiments, the disclosure provides a method of treating disease, such as a viral infection, comprising administering to a subject in need of such treatment an effective amount of a compound of the present disclosure, or a pharmaceutically acceptable salt thereof. In some embodiments, the viral infection may be drug-resistant to one or more of nirmatrelvir, ritonavir, GRL-0617, remdesivir, favipiravir, ribavirin, monoclonal antibodies, dexamethasone, interferon, umifenovir, oseltamivir, lopinavir, and ritonavir. In some embodiments, the disclosure provides a method of treating disease, such as a drug-resistant viral infection (e.g., a nirmatrelvir-resistant viral infection, for example, caused by SARS-CoV-2), comprising administering to a subject in need of such treatment an effective amount of a compound of the present disclosure, or a pharmaceutically acceptable salt thereof.

In one aspect, the compounds and pharmaceutical compositions of the invention specifically target SC2M^{Pro}. Thus, these compounds and pharmaceutical compositions can be used to prevent, reverse, slow, or inhibit the activity of this protease. In preferred embodiments, methods of treatment include treating viral infections. In other embodiments, methods are for treating viral infections caused by SARS-CoV-2 (e.g., COVID-19).

In the inhibitory methods of the invention, an “effective amount” means an amount sufficient to inhibit the target protein. Measuring such target modulation may be performed by routine analytical methods such as those described below. Such modulation is useful in a variety of settings, including in vitro assays.

In treatment methods according to the invention, an “effective amount” means an amount or dose sufficient to generally bring about the desired therapeutic benefit in subjects needing such treatment. Effective amounts or doses of the compounds of the invention may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about from about 0.1 mg to 1 g daily, or about 1 mg to 50 mg daily, or about 50 to 250 mg daily, or about 250 mg to 1 g daily. The total dosage may be given in single or divided dosage units (e.g., BID, TID, QID).

Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms. Patients may also require chronic treatment on a long-term basis.

### Drug Combinations

The inventive compounds described herein may be used in pharmaceutical compositions or methods in combination with one or more additional active ingredients in the treatment of the diseases and disorders described herein. Further additional active ingredients include other therapeutics or agents that mitigate adverse effects of therapies for the intended disease targets. Such combinations may serve to increase efficacy, ameliorate other disease symptoms, decrease one or more side effects, or decrease the required dose of an inventive compound. The additional active ingredients may be administered in a separate pharmaceutical composition from a compound of the present invention or may be included with a compound of the present invention in a single pharmaceutical composition. The additional active ingredients may be administered simultaneously with, prior to, or after administration of a compound of the present invention.

Combination agents include additional active ingredients are those that are known or discovered to be effective in treating the diseases and disorders described herein, including those active against another target associated with the disease. For example, compositions and formulations of the invention, as well as methods of treatment, can further comprise other drugs or pharmaceuticals, e.g., other active agents useful for treating or palliative for the target diseases or related symptoms or conditions. For viral infections, additional such agents include, but are not limited to, nirmatrelvir, ritonavir, GRL-0617, remdesivir, favipiravir, ribavirin, monoclonal antibodies, dexamethasone, interferon, umifenovir, oseltamivir, lopinavir, and ritonavir. The pharmaceutical compositions described herein may additionally comprise one or more of such active agents, and methods of treatment may additionally comprise administering an effective amount of one or more of such active agents.

In some embodiments, a compound of the present disclosure and an antiviral, or a combination of antivirals, provide a synergistic effect. For example, a combination of a compound of the present disclosure and nirmatrelvir, ritonavir, GRL-0617, or a combination thereof provide a synergistic effect. The synergistic effect can be a decrease in viral load, for example.

### Chemical Synthesis

Exemplary chemical entities useful in methods of the description will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Furthermore, one of skill in the art will recognize that the transformations shown in the schemes below may be performed in any order that is compatible with the functionality of the particular pendant groups.

### Abbreviations

The examples described herein use materials, including but not limited to, those described by the following abbreviations known to those skilled in the art:

| | |
|---|---|
| g | grams |
| eq | equivalents |
| mmol | millimoles |
| mL | milliliters |
| MHz | megahertz |
| Ppm | parts per million |
| Δ | chemical shift |
| s | singlet |
| d | doublet |
| t | triplet |
| q | quartet |
| qu | quintet |
| br | broad |
| m | multiplet |
| Hz | hertz |
| RT | room temperature |
| ° C. | degrees Celsius |
| R_{f} | retardation factor |
| N | normal |
| J | coupling constant |
| MS | mass spectrum |
| m/z | mass-to-charge ratio |
| min | minutes |
| hr | hours |
| TLC | thin layer chromatography |
| M | molar |
| μM or uM | micromolar |
| U/mL | units of activity per milliliter |
| DMSO-d₆ | deuterated dimethyl sulfoxide |
| AcOH | acetic acid |
| MeCN (ACN) | acetonitrile |
| MeOH | methanol |
| EtOH | ethanol |
| EtOAc or EA | ethyl acetate |
| DMF | dimethylformamide |
| DIPEA or | N,N-diisopropylethylamine |
| DIEA or | |
| Hunig's Base | |
| THF | tetrahydrofuran |
| DMAP | 4-(dimethylamino)pyridine |
| DMP | Dess-Martin periodinane |
| DMSO | dimethyl sulfoxide |
| TFA | trifluoroacetic acid |
| TMS | trimethylsilane |
| TFE | trifluoroethylene |
| DCM | dichloromethane |
| DMF | N,N-dimethylformamide |
| Cbz | Benzyl formate |
| Boc | tert -butoxycarbonyl |
| mCPBA | Meta-Chloroperoxy benzoic acid |
| PEG | polyethylene glycol |
| HMDS | hexamethyldisilazide |
| THF | tetrahydrofuran |
| HATU | Hexafluorophosphate Azabenzotriazole Tetramethyl Uronium |
| HBTU | (2-(1H-benzotriazol-1-yl)-1,1,3,3-tetramethyluronium |
| | hexafluorophosphate |
| COMU | [[(Z)-(1-cyano-2-ethoxy-2-oxoethylidene)amino]oxy-morpholin-4- |
| | ylmethylidene]-dimethylazanium; hexafluorophosphate |
| DBU | 1,8-diazabicyclo(5.4.0)undec-7-ene |
| IC₅₀ | Inhibitory concentration for half-maximal signal in biochemical assay |
| EC₅₀ | Effective concentration for half-maximal signal in proliferation assays |
| CC₅₀ | Cytotoxicity concentration for half-maximal signal in cell viability assays. |

All reagents and solvents for the synthesis were purchased from commercial sources and used without purification. All glassware was flame-dried prior to use. Thin-layer chromatography (TLC) was carried out on aluminum plates coated with 60 F254 silica gel. TLC plates were visualized under UV light (254 or 365 nm) or stained with 5% phosphomolybdic acid. Normal phase column chromatography was carried out using a Yamazen Small Flash AKROS system. NMR spectra were recorded on a Bruker AVANCE Neo 400 MHz spectrometer in specified deuterated solvents. The purity of the final MPDs was assessed by LC-MS to confirm >95% purity. Analytical liquid chromatography-mass spectrometry was performed on a PHENOMENEX C18 Column (150×2.00 mm 5 micron, gradient from 10% to 100% B [A=10 mmol/L HCOONH4/H₂O, B=MeOH], flow rate: 0.3 mL/min) using a Thermo Scientific Ultimate 3000 with a UV-detector (detection at 215 nm), equipped with a Thermo Scientific Orbitrap Q Exactive Focus System.

The proposed targets can be prepared via the conventional chemistry or following the general schemes as shown below.

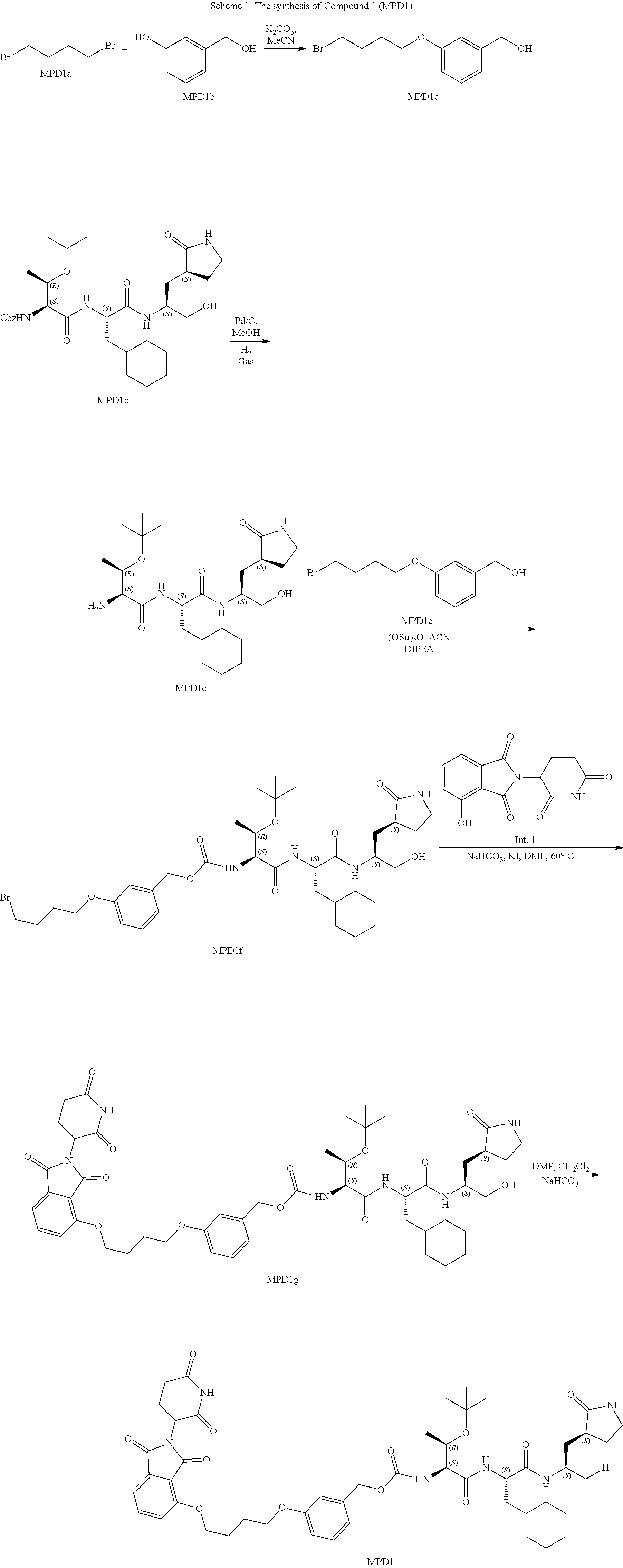

Synthesis of (3-(4-bromobutoxy)phenyl)methanol (MPD1c): To a solution of MPD1a (13.05 g, 60 mmol) and MPD1b (1.5 g, 12.09 mmol) in acetonitrile (80 mL) was added K₂CO₃ (8.3 g, 60 mmol). The reaction mixture was then heated to reflux overnight. The reaction mixture was then evaporated in vacuo, the residue was then diluted with H₂O and DCM. The organic layer was separated, washed with saturated brine solution, dried over anhydrous Na₂SO₄, and concentrated in vacuo. The residue was purified by flash chromatography (0-10% EtOAc in hexanes as eluent) to yield MPD1c as white solid (2.5 g, 66%). ¹H NMR (400 MHz, CDCl₃) δ 7.23-7.14 (m, 1H), 6.89-6.82 (m, 2H), 6.78-6.70 (m, 1H), 4.59 (s, 2H), 3.93 (t, J=6.0 Hz, 2H), 3.42 (t, J=6.6 Hz, 2H), 2.06-1.93 (m, 2H), 1.92-1.81 (m, 2H).

Synthesis of (2S,3R)-2-amino-3-(tert-butoxy)-N—((S)-3-cyclohexyl-1-(((S)-1-hydroxy-3-((S)-2-oxopyrrolidin-3-yl)propan-2-yl)amino)-1-oxopropan-2-yl)butanamide (MPD1e): To a solution of MPD1d (602 mg, 1 mmol) in methanol (20 mL) was added 10% Pd/C (100 mg). The reaction flask was sealed and the air inside was removed under Schlenk line. A hydrogen balloon was then attached to the reaction flask and the reaction mixture was stirred under room temperature for 3 h. After the completion of deprotection, the reaction mixture was filtered with a pad of Celite. The filtrate was concentrated in vacuo and the residue was used for the next step without further purification.

Synthesis of 3-(4-bromobutoxy)benzyl ((2S,3R)-3-(tert-butoxy)-1-(((S)-3-cyclohexyl-1-(((S)-1-hydroxy-3-((S)-2-oxopyrrolidin-3-yl)propan-2-yl)amino)-1-oxopropan-2-yl)amino)-1-oxobutan-2-yl)carbamate (MPD1f): To a solution of MPD1c (200 mg, 0.775 mmol) in anhydrous ACN (5 mL) was added NMM (236 mg, 2.32 mmol) at 0° C. bis(2,5-dioxopyrrolidin-1-yl) carbonate (278 mg, 1.08 mmol) was slowly added to the solution at the same temperature. The reaction mixture was then allowed to warm up to room temperature and stirred overnight. Then Compound 5 (362 mg, 0.775) was added to reaction and stirred for 2 h. Solvent was removed vacuum and 20 ml Ethyl acetate added to residue. The reaction mixture was then washed by 1 M HCl (2×10 mL). The organic layer was separated, dried over anhydrous Na₂SO₄, and concentrated in vacuo. The residue was purified by flash chromatography (50-100% EtOAc in hexanes as eluent) to yield MPD1f as white solid (250 mg, 49%). ¹H NMR (400 MHz, CDCl₃) δ 7.49 (d, J=7.9 Hz, 1H), 7.34 (d, J=7.7 Hz, 1H), 7.21-7.14 (m, 1H), 6.88-6.73 (m, 3H), 6.19 (s, 1H), 6.06 (d, J=5.3 Hz, 1H), 5.08-4.92 (m, 2H), 4.34 (td, J=8.5, 5.6 Hz, 1H), 4.09 (dd, J=6.1, 3.3 Hz, 2H), 4.00-3.84 (m, 3H), 3.60-3.49 (m, 2H), 3.34 (t, J=6.8 Hz, 2H), 3.28-3.17 (m, 2H), 2.38-2.26 (m, 2H), 2.05-1.93 (m, 1H), 1.85-1.21 (m, 21H), 1.19 (s, 9H), 1.14-1.06 (m, 3H), 1.01 (d, J=6.1 Hz, 4H), 0.96-0.75 (m, 2H).

Synthesis of 3-(4-((2-(2,6-dioxopiperidin-3-yl)-1,3-dioxoisoindolin-4-yl)oxy)butoxy)benzyl ((2S,3R)-3-(tert-butoxy)-1-(((S)-3-cyclohexyl-1-(((S)-1-hydroxy-3-((S)-2-oxopyrrolidin-3-yl)propan-2-yl)amino)-1-oxopropan-2-yl)amino)-1-oxobutan-2-yl)carbamate (MPD1g): To a solution of MPD1f (100 mg, 0.13 mmol) in DMF (2 mL) was added Int. 1 (44 mg, 0.15 mmol), NaHCO₃ (23 mg, 0.26 mmol), KI (2 mg, 0.013 mmol). The reaction mixture was then stirred at 60° C. for 24 h. The reaction mixture was then diluted with EtOAc (20 mL), washed with H₂O (20 mL) and saturated brine solution (20 mL). The organic layer was separated, dried over anhydrous Na₂SO₄, and concentrated in vacuo. The residue was purified with flash chromatography (5-10% methanol in EtOAc as eluent) to yield MPD1g as white solid (85 mg, 55%). ¹H NMR (400 MHz, DMSO) δ 11.10 (s, 1H), 7.82 (dt, J=8.5, 7.0 Hz, 2H), 7.71 (d, J=8.9 Hz, 1H), 7.55-7.48 (m, 2H), 7.44 (d, J=7.2 Hz, 1H), 7.25 (t, J=7.8 Hz, 1H), 6.95-6.81 (m, 4H), 5.12-4.95 (m, 3H), 4.66 (t, J=5.6 Hz, 1H), 4.30 (q, J=7.6 Hz, 1H), 4.21 (t, J=6.4 Hz, 2H), 4.01 (dd, J=9.1, 4.0 Hz, 1H), 3.97 (t, J=2.1 Hz, 2H), 3.90-3.83 (m, 1H), 3.76 (s, 1H), 3.26-3.11 (m, 2H), 3.09-2.99 (m, 1H), 2.88 (ddd, J=17.4, 14.1, 5.4 Hz, 1H), 2.58 (d, J=13.8 Hz, 1H), 2.25-2.10 (m, 2H), 2.05-1.99 (m, 1H), 1.86-1.24 (m, 23H), 1.10 (s, 9H), 1.02 (d, J=6.2 Hz, 3H), 0.88 (dt, J=25.2, 13.9 Hz, 2H).

Synthesis of 3-(4-((2-(2,6-dioxopiperidin-3-yl)-1,3-dioxoisoindolin-4-yl)oxy)butoxy)benzyl ((2S,3R)-3-(tert-butoxy)-1-(((S)-3-cyclohexyl-1-oxo-1-(((S)-1-oxo-3-((S)-2-oxopyrrolidin-3-yl)propan-2-yl)amino)propan-2-yl)amino)-1-oxobutan-2-yl)carbamate (1; MPD1): To a solution of MPD1g (80 mg, 0.084 mmol) in anhydrous DCM (5 mL) was added Dess-Martin periodinane (44 mg, 0.101 mmol). The reaction mixture was stirred at room temperature for 5 h. Then the reaction was quenched by the addition of 10% Na₂S₂O₃ in saturated NaHCO₃ solution (10 mL). The mixture was further stirred until the organic layer became clear. The organic layer was then separated, dried with anhydrous Na₂SO₄, and concentrated in vacuo. The residue was then purified with flash chromatography (0-10 methanol in DCM as eluent) to yield Compound 1 (MPD1) as white solid (60 mg, 73%). Analytical data is provided in the table below.

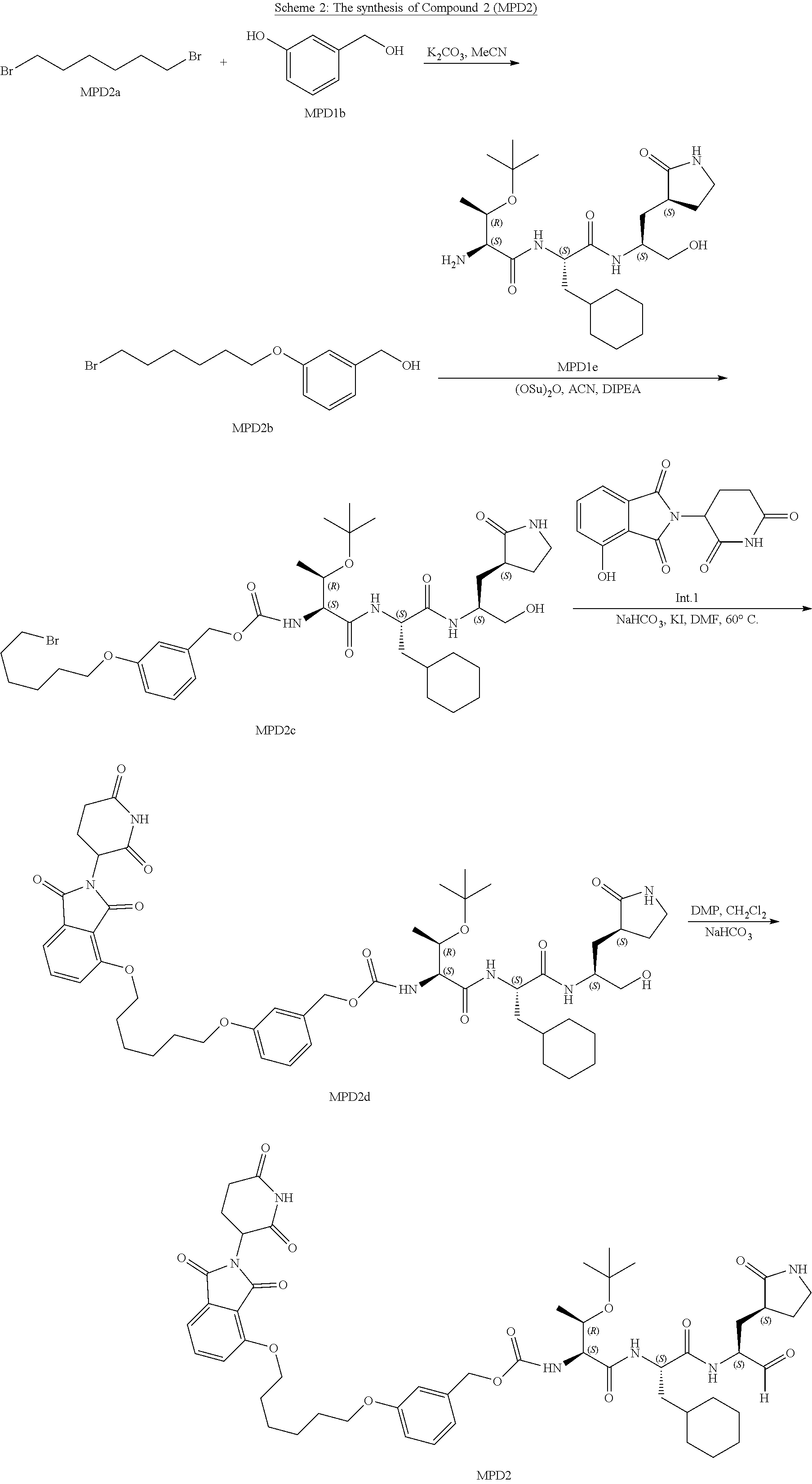

Synthesis of (3-((6-bromohexyl)oxy)phenyl)methanol (MPD2b): MPD2b was prepared as a white solid following a similar procedure to MPD1c (yield 77%). ¹H NMR (400 MHz, Chloroform-d) δ 7.36-7.24 (m, 1H), 7.00-6.91 (m, 2H), 6.85 (dd, J=8.4, 2.5 Hz, 1H), 4.69 (s, 2H), 4.00 (t, J=6.4 Hz, 2H), 3.45 (t, J=6.8 Hz, 2H), 1.92 (p, J=6.9 Hz, 2H), 1.83 (p, J=6.8 Hz, 2H), 1.59-1.47 (m, 4H).

Synthesis of 3-((6-bromohexyl)oxy)benzyl ((2S,3R)-3-(tert-butoxy)-1-(((S)-3-cyclohexyl-1-(((S)-1-hydroxy-3-((S)-2-oxopyrrolidin-3-yl)propan-2-yl)amino)-1-oxopropan-2-yl)amino)-1-oxobutan-2-yl)carbamate (MPD2c): MPD2c was prepared as a white solid following a similar procedure to MPD1f (yield 50%). ¹H NMR (400 MHz, DMSO-d₆) δ 7.84 (d, J=8.0 Hz, 1H), 7.71 (d, J=8.9 Hz, 1H), 7.53 (s, 1H), 7.26 (t, J=7.8 Hz, 1H), 6.99-6.90 (m, 2H), 6.90-6.78 (m, 2H), 5.12-4.90 (m, 2H), 4.65 (t, J=5.6 Hz, 1H), 4.31 (q, J=7.7 Hz, 1H), 4.01 (dd, J=9.0, 4.1 Hz, 1H), 3.96 (t, J=6.4 Hz, 2H), 3.87 (dd, J=6.4, 4.4 Hz, 1H), 3.75 (d, J=7.2 Hz, 1H), 3.55 (t, J=6.7 Hz, 2H), 3.21 (dt, J=10.4, 6.4 Hz, 1H), 3.14 (t, J=9.1 Hz, 1H), 3.10-2.97 (m, 1H), 2.25-2.08 (m, 2H), 1.88-1.78 (m, 2H), 1.77-1.51 (m, 9H), 1.51-1.35 (m, 7H), 1.33-1.22 (m, 1H), 1.19-1.06 (m, 12H), 1.03 (d, J=6.2 Hz, 3H), 0.96-0.77 (m, 3H).

Synthesis of 3-((6-((2-(2,6-dioxopiperidin-3-yl)-1,3-dioxoisoindolin-4-yl)oxy)hexyl)oxy)benzyl ((2S,3R)-3-(tert-butoxy)-1-(((S)-3-cyclohexyl-1-(((S)-1-hydroxy-3-((S)-2-oxopyrrolidin-3-yl)propan-2-yl)amino)-1-oxopropan-2-yl)amino)-1-oxobutan-2-yl)carbamate (MPD2d): MPD2d was prepared as a white solid following a similar procedure to MPD1g (yield 64%). ¹H NMR (400 MHz, DMSO-d₆) δ 11.09 (s, 1H), 7.90-7.75 (m, 2H), 7.70 (d, J=8.9 Hz, 1H), 7.55-7.47 (m, 2H), 7.44 (d, J=7.2 Hz, 1H), 7.24 (t, J=7.8 Hz, 1H), 6.96-6.79 (m, 4H), 5.11-4.94 (m, 3H), 4.74-4.63 (m, 1H), 4.30 (q, J=7.7 Hz, 1H), 4.22 (t, J=6.2 Hz, 2H), 4.03-3.93 (m, 3H), 3.91-3.82 (m, 1H), 3.80-3.69 (m, 1H), 3.25-3.17 (m, 2H), 3.17-3.08 (m, 1H), 3.08-2.97 (m, 1H), 2.87 (s, 2H), 2.13 (d, J=15.5 Hz, 2H), 2.08-2.01 (m, 1H), 1.86-1.64 (m, 7H), 1.65-1.33 (m, 11H), 1.14-1.04 (m, 12H), 1.01 (d, J=6.2 Hz, 3H), 0.93-0.75 (m, 4H).

Synthesis of 3-((6-((2-(2,6-dioxopiperidin-3-yl)-1,3-dioxoisoindolin-4-yl)oxy)hexyl)oxy)benzyl ((2S,3R)-3-(tert-butoxy)-1-(((S)-3-cyclohexyl-1-oxo-1-(((S)-1-oxo-3-((S)-2-oxopyrrolidin-3-yl)propan-2-yl)amino)propan-2-yl)amino)-1-oxobutan-2-yl)carbamate (2; MPD2): Compound 2 (MPD2) was prepared as a white solid following a similar procedure to Compound 1 (MPD1) (yield 60%). Analytical data is provided in the table below.

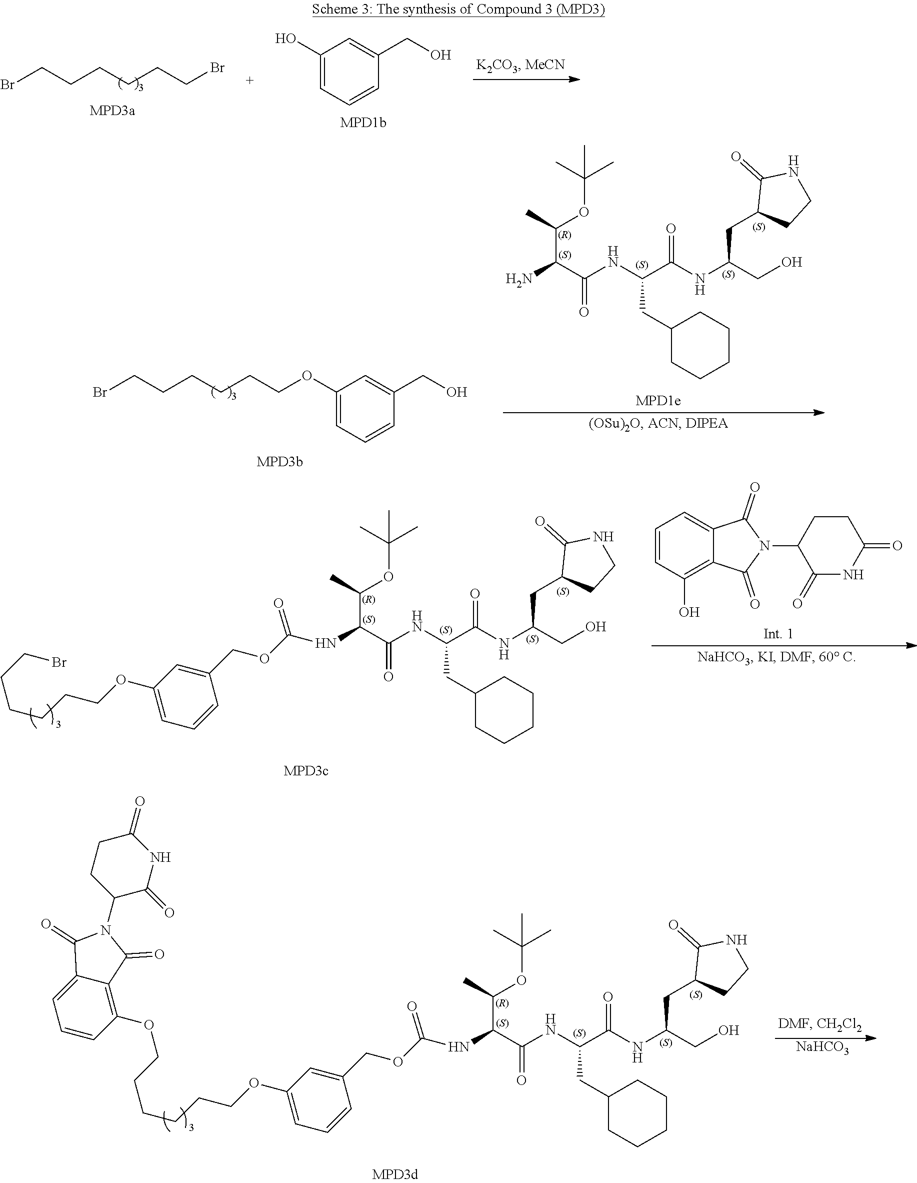

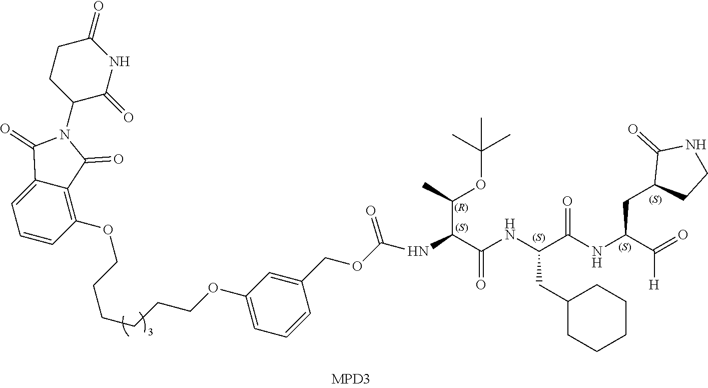

Synthesis of (3-((8-bromooctyl)oxy)phenyl)methanol (MPD3b): MPD3b was prepared as a white solid following a similar procedure to MPD1c (yield 66%). ¹H NMR (400 MHz, CDCl₃) δ 7.23-7.14 (m, 1H), 6.88-6.82 (m, 2H), 6.75 (ddd, J=8.2, 2.6, 1.0 Hz, 1H), 4.59 (s, 2H), 3.89 (t, J=6.5 Hz, 2H), 3.34 (t, J=6.8 Hz, 2H), 1.79 (p, J=6.9 Hz, 2H), 1.71 (dq, J=8.1, 6.5 Hz, 2H), 1.46-1.22 (m, 8H).

Synthesis of 3-((8-bromooctyl)oxy)benzyl ((2S,3R)-3-(tert-butoxy)-1-(((S)-3-cyclohexyl-1-(((S)-1-hydroxy-3-((S)-2-oxopyrrolidin-3-yl)propan-2-yl)amino)-1-oxopropan-2-yl)amino)-1-oxobutan-2-yl)carbamate (MPD3c): MPD3c was prepared as a white solid following a similar procedure to MPD1f (yield 49%). ¹H NMR (400 MHz, CDCl₃) δ 7.51 (d, J=7.8 Hz, 1H), 7.35 (dd, J=7.8, 2.4 Hz, 1H), 7.23-7.14 (m, 1H), 6.90-6.73 (m, 3H), 6.24-6.03 (m, 2H), 5.07-4.92 (m, 2H), 4.34 (td, J=8.5, 5.7 Hz, 1H), 4.07 (dd, J=19.3, 6.3 Hz, 2H), 3.93 (t, J=6.0 Hz, 3H), 3.54 (t, J=3.4 Hz, 1H), 3.42 (t, J=6.6 Hz, 2H), 3.22 (d, J=8.3 Hz, 2H), 2.38-2.27 (m, 2H), 1.99 (ddt, J=10.2, 7.2, 5.0 Hz, 3H), 1.87 (dq, J=9.7, 6.1 Hz, 2H), 1.78-1.43 (m, 9H), 1.19 (s, 9H), 1.15-1.05 (m, 3H), 1.01 (d, J=6.0 Hz, 3H), 0.96-0.76 (m, 2H).

Synthesis of 3-((8-((2-(2,6-dioxopiperidin-3-yl)-1,3-dioxoisoindolin-4-yl)oxy)octyl)oxy)benzyl ((2S,3R)-3-(tert-butoxy)-1-(((S)-3-cyclohexyl-1-(((S)-1-hydroxy-3-((S)-2-oxopyrrolidin-3-yl)propan-2-yl)amino)-1-oxopropan-2-yl)amino)-1-oxobutan-2-yl)carbamate (MPD3d): MPD3d was prepared as a white solid following a similar procedure to MPD1g (yield 45%). ¹H NMR (400 MHz, DMSO) δ 11.03 (s, 1H), 7.75 (dd, J=8.6, 7.3 Hz, 2H), 7.63 (d, J=9.0 Hz, 1H), 7.49-7.42 (m, 2H), 7.38 (d, J=7.2 Hz, 1H), 7.18 (t, J=7.9 Hz, 1H), 6.90-6.73 (m, 4H), 5.01 (dd, J=12.8, 5.4 Hz, 1H), 4.98-4.88 (m, 2H), 4.58 (t, J=5.6 Hz, 1H), 4.23 (d, J=7.1 Hz, 3H), 4.02-3.90 (m, 4H), 3.79 (t, J=5.4 Hz, 1H), 3.68 (s, 1H), 3.18-3.11 (m, 1H), 3.06 (t, J=9.0 Hz, 1H), 3.01-2.94 (m, 1H), 2.81 (ddd, J=17.7, 13.8, 5.3 Hz, 1H), 2.56-2.45 (m, 1H), 2.08 (s, 2H), 1.99-1.92 (m, 1H), 1.86 (s, 3H), 1.57 (ddd, J=62.7, 22.3, 10.9 Hz, 7H), 1.39-1.26 (m, 3H), 1.14 (d, J=30.4 Hz, 2H), 1.03 (s, 9H), 1.00-0.92 (m, 6H), 0.77 (t, J=12.3 Hz, 2H).

Synthesis of 3-((8-((2-(2,6-dioxopiperidin-3-yl)-1,3-dioxoisoindolin-4-yl)oxy)octyl)oxy)benzyl ((2S,3R)-3-(tert-butoxy)-1-(((S)-3-cyclohexyl-1-oxo-1-(((S)-1-oxo-3-((S)-2-oxopyrrolidin-3-yl)propan-2-yl)amino)propan-2-yl)amino)-1-oxobutan-2-yl)carbamate (3; MPD3): Compound 3 (MPD3) was prepared as a white solid following a similar procedure to Compound 1 (MPD1) (yield 73%). Analytical data is provided in the table below.

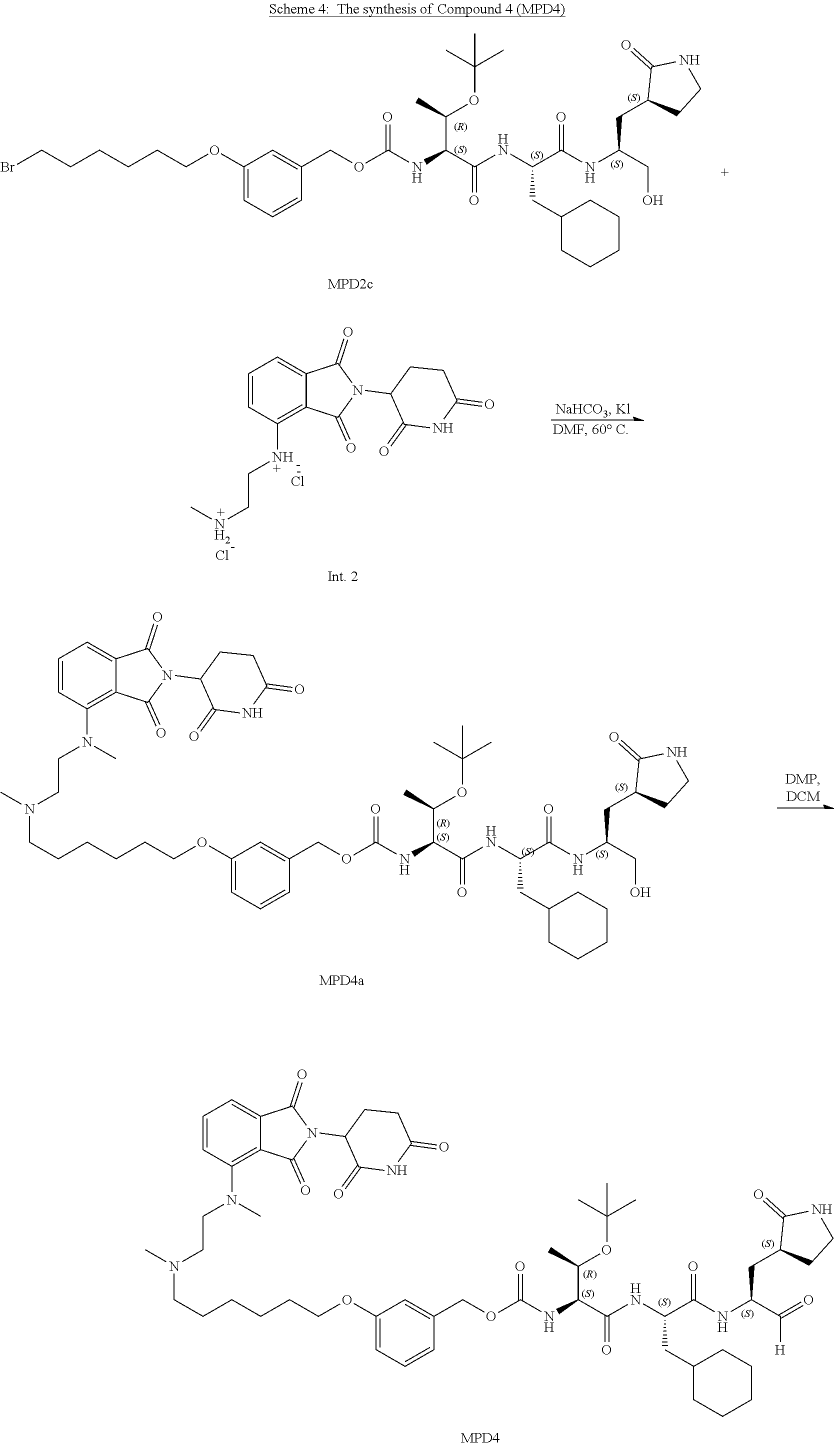

Synthesis of 3-((6-((2-((2-(2,6-dioxopiperidin-3-yl)-1,3-dioxoisoindolin-4-yl)(methyl)amino)ethyl)(methyl)amino)hexyl)oxy)benzyl ((2S,3R)-3-(tert-butoxy)-1-(((S)-3-cyclohexyl-1-(((S)-1-hydroxy-3-((S)-2-oxopyrrolidin-3-yl)propan-2-yl)amino)-1-oxopropan-2-yl)amino)-1-oxobutan-2-yl)carbamate (MPD4a): MPD4a was prepared as a white solid following a similar procedure to MPD1g (yield 35%).

Synthesis of 3-((6-((2-((2-(2,6-dioxopiperidin-3-yl)-1,3-dioxoisoindolin-4-yl)(methyl)amino)ethyl)(methyl)amino)hexyl)oxy)benzyl ((2S,3R)-3-(tert-butoxy)-1-(((S)-3-cyclohexyl-1-oxo-1-(((S)-1-oxo-3-((S)-2-oxopyrrolidin-3-yl)propan-2-yl)amino)propan-2-yl)amino)-1-oxobutan-2-yl)carbamate (4; MPD4): Compound 4 (MPD4) was prepared as a white solid following a similar procedure to Compound 1 (MPD1) (yield 56%). Analytical data is provided in the table below.

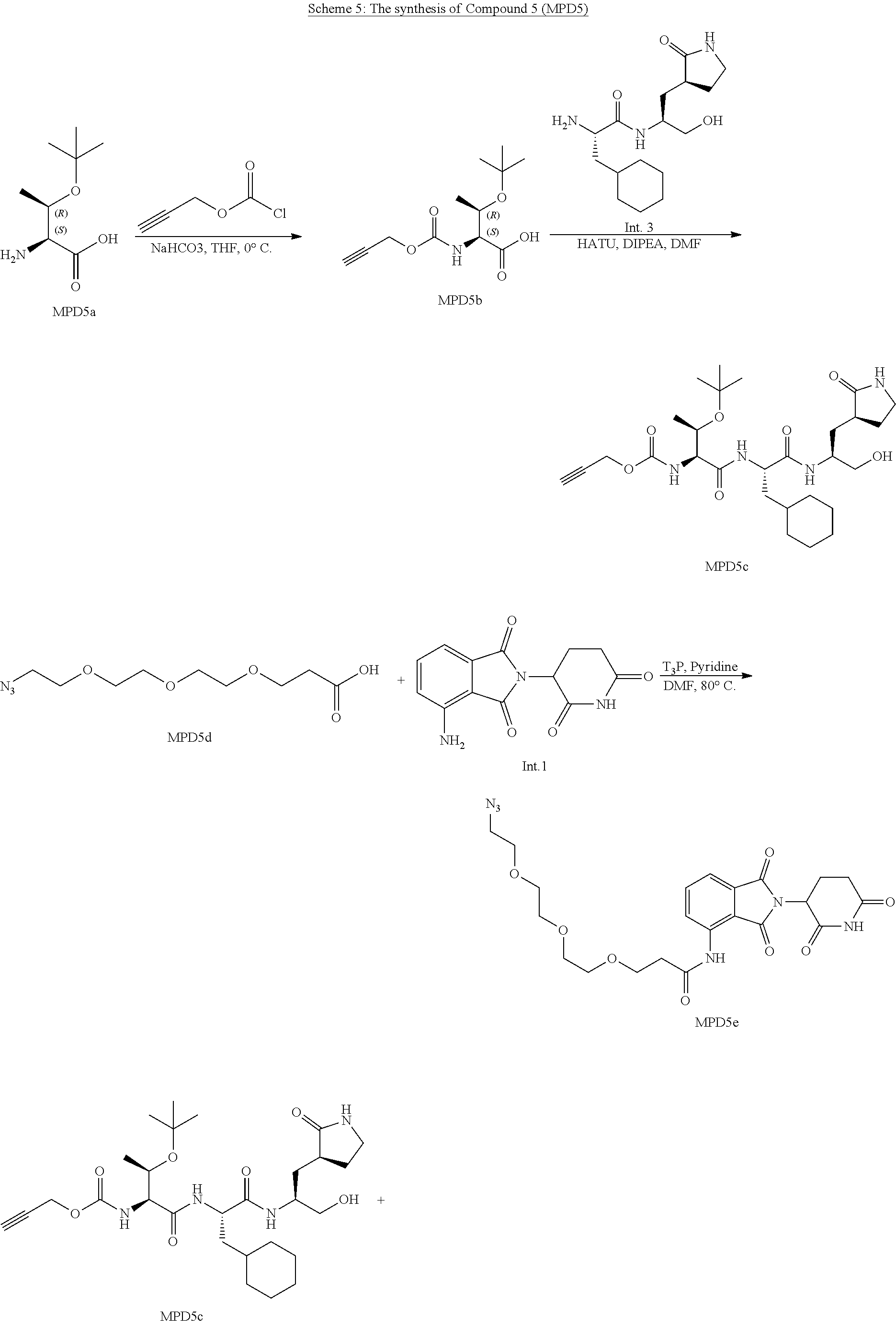

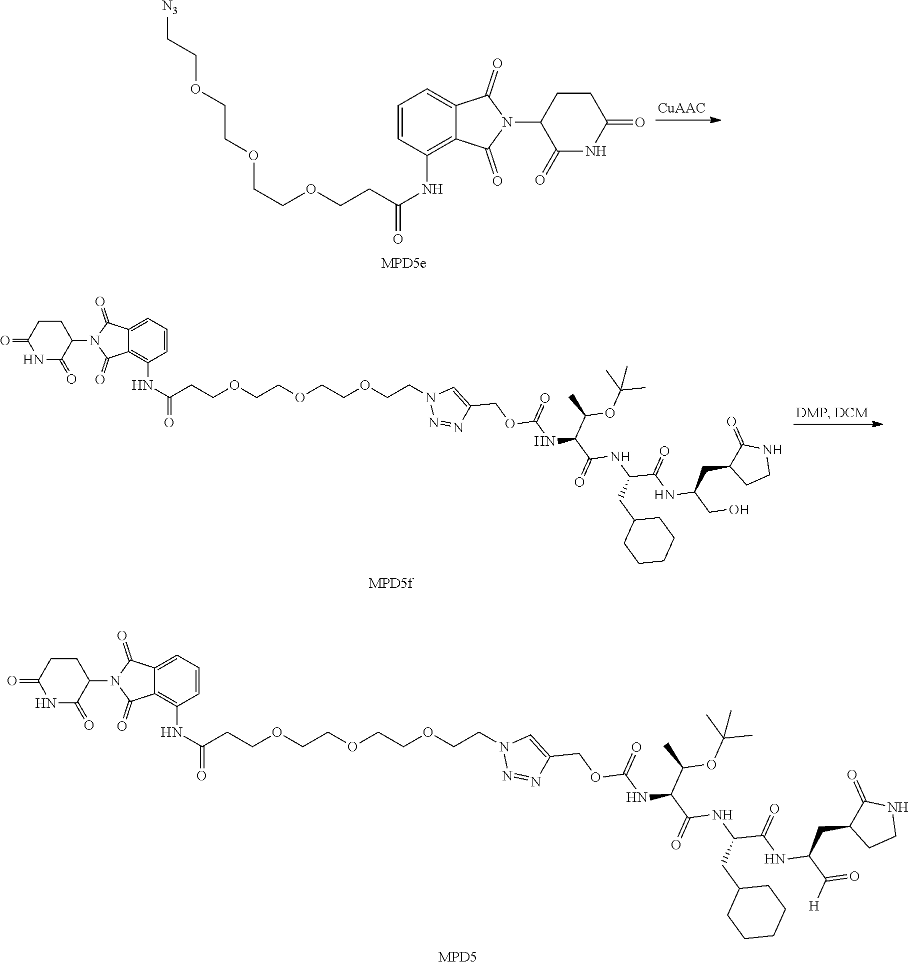 [SMILES: [N-]=[N+]=NCCOCCOCCOCCC(=O)Nc1cccc2c1C(=O)N(C1CCC(=O)NC1=O)C2=O ; C[C@@H](OC(C)(C)C)[C@H](NC(=O)OCc1cn(CCOCCOCCOCCC(=O)Nc2cccc3c2C(=O)N(C2CCC(=O)NC2=O)C3=O)nn1)C(=O)N[C@@H](CC1CCCCC1)C(=O)N[C@H](CO)C[C@@H]1CCNC1=O ; C[C@@H](OC(C)(C)C)[C@H](NC(=O)OCc1cn(CCOCCOCCOCCC(=O)Nc2cccc3c2C(=O)N(C2CCC(=O)NC2=O)C3=O)nn1)C(=O)N[C@@H](CC1CCCCC1)C(=O)N[C@H](C=O)C[C@@H]1CCNC1=O]

Synthesis of O-(tert-butyl)-N-((prop-2-yn-1-yloxy)carbonyl)-L-threonine (MPD5b): Dissolved O-(tert-butyl)-L-threonine (MPD5a) (2.0 g, 11.41 mmol) in 40 mL saturated sodium bicarbonate solution, The temperature was lowered to 0° C., a THF solution of propargyl chloroformate (13.7 mmol) was added dropwise, and the mixture was stirred vigorously. After dropping, the reaction solution was heated to 25° C., and the reaction was continued for 2 hours. After the reaction is complete, adjust the pH of the reaction solution to 11 with saturated sodium hydroxide solution. Wash the water phase with 40 mL ethyl acetate three times, after washing, adjust the pH of the reaction solution to 2 with concentrated hydrochloric acid. The aqueous phase was extracted three times with 50 mL of ethyl acetate, the organic phases were combined and dried with anhydrous sodium sulfate, The organic solvent was evaporated under reduced pressure, 2.3 g of white solid was obtained with a yield of 78%. ¹H NMR (400 MHz, CDCl₃) δ 5.76 (d, J=6.8 Hz, 1H), 4.72 (t, J=2.9 Hz, 2H), 4.32 (ddt, J=15.9, 6.5, 3.4 Hz, 2H), 2.51 (q, J=3.1 Hz, 1H), 1.27 (s, 9H), 1.19 (d, J=6.3 Hz, 3H). ¹³C NMR (101 MHz, CDCl₃) δ 172.80, 155.41, 77.80, 76.33, 74.99, 66.68, 60.45, 58.71, 28.10, 18.42.

Synthesis of Prop-2-yn-1-yl ((2S,3S)-3-(tert-butoxy)-1-(((S)-3-cyclohexyl-1-(((S)-1-hydroxy-3-((S)-2-oxopyrrolidin-3-yl)propan-2-yl)amino)-1-oxopropan-2-yl)amino)-1-oxobutan-2-yl)carbamate (MPD5c): The acid compound MPD5b (0.181 g, 0.70 mmol) and the amine Int. 3 (0.2 g, 0.64 mmol) were dissolved in dry DMF (10 mL) and the reaction was cooled to 0° C. HATU (317 mg, 0.83 mmol) and DIPEA (0.46 mL, 2.57 mmol) were added, and the reaction mixture was allowed warm up to room temperature and stirred for 12 h. The mixture was then poured into water (50 mL) and extracted with ethyl acetate (4×20 mL). The organic layer was washed with aqueous hydrochloric acid 10% v/v (2×20 mL), saturated aqueous NaHCO₃ (2×20 mL), brine (2×20 mL) and dried over Na₂SO₄. The organic phase was evaporated to dryness and the crude material purified by silica gel column chromatography (0-10% MeOH in CH₂Cl₂ as the eluent) to afford MPD5c white gummy solid (180 mg, 56%). ¹H NMR (400 MHz, CDCl₃) δ 7.57 (d, J=7.9 Hz, 1H), 7.37 (d, J=7.6 Hz, 1H), 6.26 (d, J=5.2 Hz, 1H), 6.10 (s, 1H), 4.62 (qd, J=15.6, 2.5 Hz, 2H), 4.34 (q, J=7.7 Hz, 1H), 4.08 (d, J=5.5 Hz, 2H), 3.95 (s, 1H), 3.64-3.51 (m, 2H), 3.31-3.20 (m, 2H), 2.44 (t, J=2.4 Hz, 1H), 2.36 (d, J=8.4 Hz, 2H), 2.03 (t, J=12.6 Hz, 1H), 1.84-1.72 (m, 1H), 1.69-1.31 (m, 9H), 1.21-1.10 (m, 12H), 1.01 (d, J=5.9 Hz, 3H), 0.85 (dq, J=22.5, 11.2 Hz, 2H). ¹³C NMR (101 MHz, CDCl₃) δ 181.05, 172.74, 169.58, 155.51, 78.08, 75.49, 74.95, 66.69, 65.84, 59.15, 55.45, 52.78, 51.84, 50.30, 40.57, 39.68, 38.25, 34.22, 33.59, 32.64, 28.52, 28.23, 26.36, 26.22, 26.03, 18.64.

Synthesis of 3-(2-(2-(2-azidoethoxy)ethoxy)ethoxy)-N-(2-(2,6-dioxopiperidin-3-yl)-1,3-dioxoisoindolin-4-yl)propenamide (MPD5e): In a 50 mL round-bottom flask, 4-amino-2-(2,6-dioxo-3-piperidyl)-isoindoline-1,3-dione (Int.1) (500 mg, 1 eq) was dissolved in DMF (5 mL) and cooled to 0° C. in an ice bath. 3-{2-[2-(2-Azidoethoxy)-ethoxy]ethoxy}propanoic acid (MPD5d) (905 mg, 2 eq) was added dropwise. Then, N-propylphosphonic acid anhydride, cyclic trimer (7.0 g, 6.5 mL, 6 eq) and pyridine (1.5 mL, 10 eq) were added. The mixture was stirred at 80° C. for 3 h. The reaction mixture concentrated and purified by by silica gel column chromatography (0-10% MeOH in CH₂Cl₂ as the eluent) to afford MPD5e white gummy solid (600 mg, 65%). ¹H NMR (400 MHz, DMSO) δ 11.21 (s, 1H), 9.94 (s, 1H), 8.61 (d, J=8.4 Hz, 1H), 7.90 (t, J=7.9 Hz, 1H), 7.68 (d, J=7.3 Hz, 1H), 5.21 (dd, J=12.7, 5.4 Hz, 1H), 3.81 (t, J=6.0 Hz, 2H), 3.73-3.51 (m, 11H), 3.43 (dt, J=14.5, 4.9 Hz, 2H), 2.96 (ddd, J=16.7, 13.7, 5.4 Hz, 1H), 2.77 (t, J=6.0 Hz, 2H), 2.71-2.57 (m, 2H), 2.18-2.09 (m, 1H).

Synthesis of (1-(2-(2-(2-(3-((2-(2,6-dioxopiperidin-3-yl)-1,3-dioxoisoindolin-4-yl)amino)-3-oxopropoxy)ethoxy)ethoxy)ethyl)-1H-1,2,3-triazol-4-yl)methyl ((2S,3R)-3-(tert-butoxy)-1-(((S)-3-cyclohexyl-1-(((S)-1-hydroxy-3-((S)-2-oxopyrrolidin-3-yl)propan-2-yl)amino)-1-oxopropan-2-yl)amino)-1-oxobutan-2-yl)carbamate (MPD5f): The alkyne compound (MPD5c) (80 mg, 0.145 mmol) and the azide (MPD5e) (73 mg, 0.145 mmol) were dissolved in tert-butanol (5 mL). Ascorbic acid (28 mg, 0.145 mmol) and copper sulfate (3.5 mg, 0.015 mmol) was dissolved in water (3 mL) and the two solutions were added together to stir overnight with monitoring by TLC. The reaction mixture was diluted with water and cooled in ice for 10 minutes, and the brown/black precipitate was collected by filtration. The precipitate was extracted with chloroform, washed with cold water (2×5 mL), dried with MgSO₄ and concentrated via rotavapor. The residue was purified by flash column chromatography (0 to 10% MeOH in CH₂Cl₂) to afford MPD5f as gummy solid 50 mg with a 33% yield. ¹H NMR (400 MHz, CDCl₃) δ 10.49 (d, J=55.1 Hz, 1H), 9.93 (d, J=10.4 Hz, 1H), 8.83 (dd, J=8.5, 4.0 Hz, 1H), 7.73-7.64 (m, 1H), 7.61-7.33 (m, 3H), 5.02 (s, 1H), 4.53-4.48 (m, 2H), 4.36-4.31 (m, 1H), 4.17 (d, J=6.7 Hz, 1H), 4.11-3.96 (m, 2H), 3.89-3.79 (m, 4H), 3.70-3.56 (m, 8H), 3.30-3.25 (m, 2H), 2.88-2.83 (m, 2H), 2.73 (q, J=6.1 Hz, 2H), 2.37-2.33 (m, 2H), 2.15-2.10 (m, 2H), 1.67 (t, J=27.1 Hz, 11H), 1.32 (s, 1H), 1.26-1.12 (m, 1H), 1.19-1.07 (m, 14H), 0.91 (d, J=15.1 Hz, 2H).

Synthesis of (1-(2-(2-(2-(3-((2-(2,6-dioxopiperidin-3-yl)-1,3-dioxoisoindolin-4-yl)amino)-3-oxopropoxy)ethoxy)ethoxy)ethyl)-1H-1,2,3-triazol-4-yl)methyl ((2S,3R)-3-(tert-butoxy)-1-(((S)-3-cyclohexyl-1-oxo-1-(((S)-1-oxo-3-((S)-2-oxopyrrolidin-3-yl)propan-2-yl)amino)propan-2-yl)amino)-1-oxobutan-2-yl)carbamate (5; MPD5): Compound 5 (MPD5) was prepared as a white solid following a similar procedure to Compound 1 (MPD1) (yield 60%). Analytical data is provided in the table below.

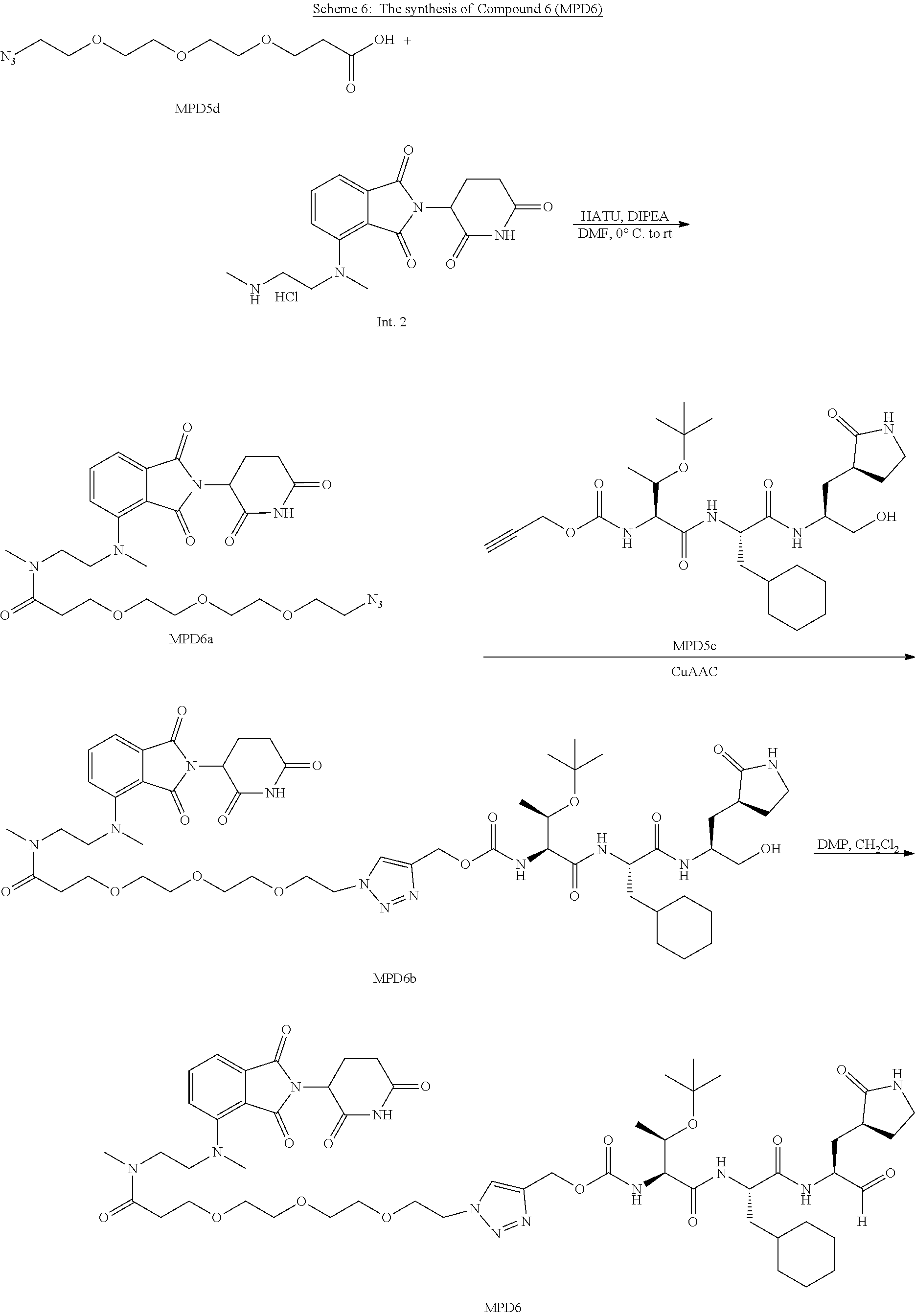

Synthesis of 3-(2-(2-(2-azidoethoxy)ethoxy)ethoxy)-N-(2-((2-(2,6-dioxopiperidin-3-yl)-1,3-dioxoisoindolin-4-yl)(methyl)amino)ethyl)-N-methylpropanamide (MPD6a): MPD6a was prepared as a white solid following a similar procedure to MPD5c (yield 66%). ¹H NMR (400 MHz, CDCl₃) δ 8.21 (s, 1H), 8.04 (s, 1H), 7.64-7.51 (m, 1H), 7.45-7.32 (m, 1H), 7.20 (dd, J=8.6, 3.8 Hz, 1H), 5.02-4.91 (m, 1H), 3.80-3.52 (m, 14H), 3.40 (q, J=4.7 Hz, 2H), 3.14 (s, 2H), 2.97 (d, J=2.0 Hz, 3H), 2.90 (d, J=0.9 Hz, 5H), 2.85-2.67 (m, 2H), 2.38 (qt, J=15.8, 6.9 Hz, 2H), 2.22-2.11 (m, 1H).

Synthesis of (1-(2-(2-(2,6-dioxopiperidin-3-yl)-1,3-dioxoisoindolin-4-yl)-5-methyl-6-oxo-9,12,15-trioxa-2,5-diazaheptadecan-17-yl)-1H-1,2,3-triazol-4-yl)methyl ((2S,3R)-3-(tert-butoxy)-1-(((S)-3-cyclohexyl-1-(((S)-1-hydroxy-3-((S)-2-oxopyrrolidin-3-yl)propan-2-yl)amino)-1-oxopropan-2-yl)amino)-1-oxobutan-2-yl)carbamate (MPD6b): MPD6b was prepared as a white solid following a similar procedure to MPD5f (yield 44%). ¹H NMR (400 MHz, CDCl₃) δ 10.03-9.40 (m, 1H), 7.66-7.26 (m, 3H), 7.08 (d, J=8.8 Hz, 1H), 5.99 (s, 1H), 5.15 (s, 1H), 4.92 (s, 1H), 4.51-4.47 (m, 2H), 4.35-4.30 (m, 1H), 4.17-4.11 (m, 1H), 4.08-3.91 (m, 2H), 3.82-3.69 (m, 4H), 3.66-3.43 (m, 14H), 3.26-3.20 (m, 2H), 3.05-2.93 (m, 3H), 2.87-2.69 (m, 5H), 2.61-2.52 (m, 1H), 2.38-2.20 (m, 4H), 2.08-2.01 (m, 1H), 1.75-1.43 (m, 10H), 1.31-1.23 (m, 1H), 1.19-1.03 (m, 17H), 0.91-0.80 (m, 2H).

Synthesis of (1-(2-(2-(2,6-dioxopiperidin-3-yl)-1,3-dioxoisoindolin-4-yl)-5-methyl-6-oxo-9,12,15-trioxa-2,5-diazaheptadecan-17-yl)-1H-1,2,3-triazol-4-yl)methyl ((2S,3R)-3-(tert-butoxy)-1-(((S)-3-cyclohexyl-1-oxo-1-(((S)-1-oxo-3-((S)-2-oxopyrrolidin-3-yl)propan-2-yl)amino)propan-2-yl)amino)-1-oxobutan-2-yl)carbamate (6; MPD6): Compound 6 (MPD6) was prepared as a white solid following a similar procedure to Compound 1 (MPD1) (yield 56%). Analytical data is provided in the table below.

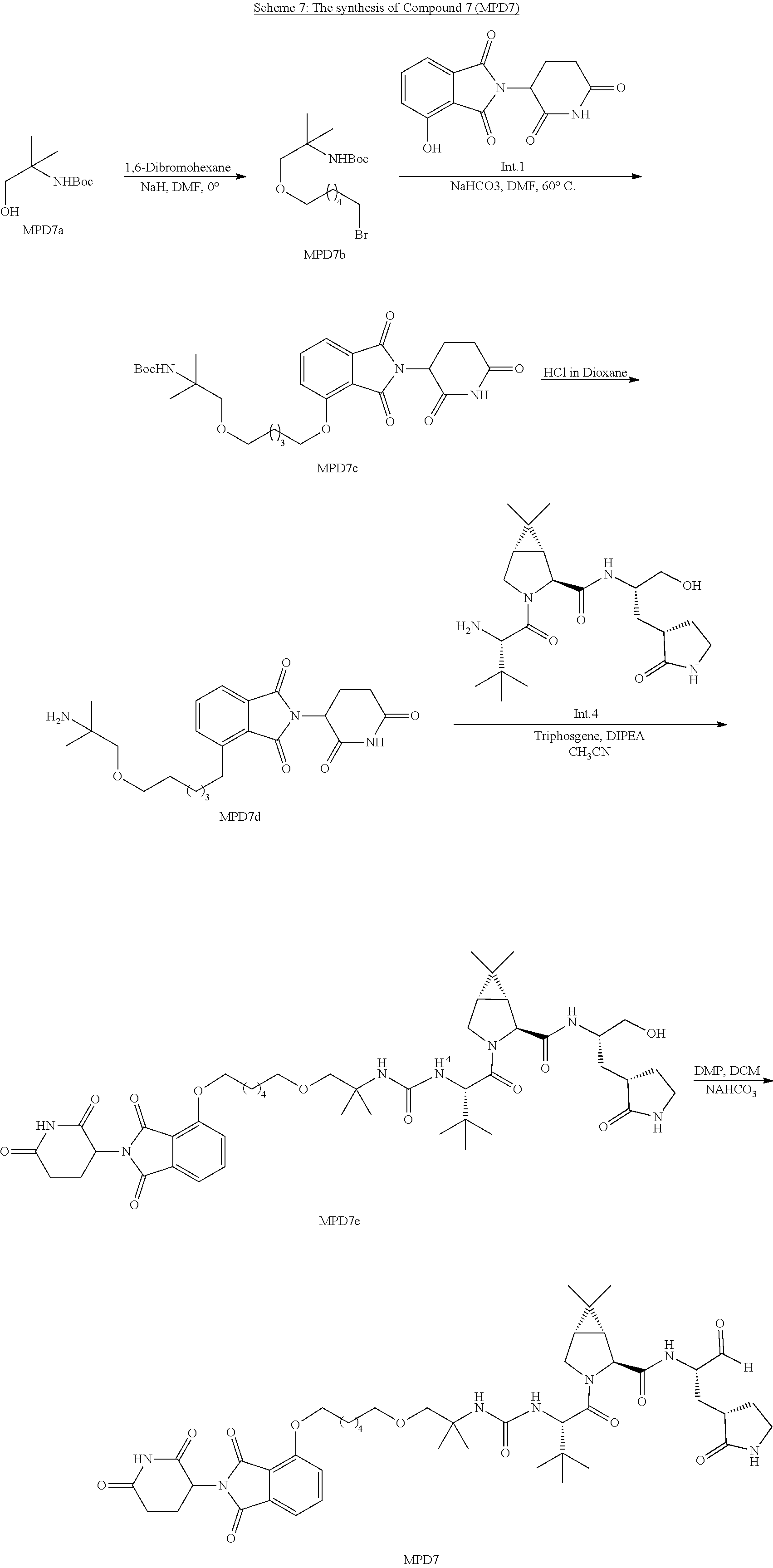

Synthesis of tert-butyl (1-((6-bromohexyl)oxy)-2-methylpropan-2-yl)carbamate (MPD7b): To a solution of NaH (100%, 116 mg, 2.91 mmol) in DMF (20 mL) was added compound MPD7a (500 mg, 7.93 mmol) at 0° C. Stir it 20 mi at same temperature. Then 1,6-Dibromo hexane (1.21 mL, 31.74 mmol) was added to reaction as drop wise at 0° C. Raise the temperature slowly to room temperature and stir for 2 h. The mixture was then poured into water (100 mL) and extracted with ethyl acetate (2×50 mL). The organic layer was washed with aqueous hydrochloric acid 10% v/v (2×50 mL), saturated aqueous NaHCO₃ (2×50 mL), brine (2×50 mL) and dried over Na₂SO₄. The organic phase was evaporated to dryness and the crude material purified by silica gel column chromatography (0-35% EtOAc in Hexanes as the eluent) to afford MPD7b white gummy solid (900 mg, 95%). ¹H NMR (400 MHz, CDCl₃) δ 4.67 (s, 1H), 3.36 (dt, J=12.0, 6.6 Hz, 4H), 3.25 (s, 2H), 1.80 (p, J=6.9 Hz, 2H), 1.57-1.46 (m, 2H), 1.46-1.27 (m, 13H), 1.21 (s, 6H). ¹³C NMR (101 MHz, CDCl₃) δ 157.80, 74.80, 58.41, 40.28, 33.81, 32.64, 29.61, 27.81, 26.16, 25.30.

Synthesis of tert-butyl (1-((6-((2-(2,6-dioxopiperidin-3-yl)-1,3-dioxoisoindolin-4-yl)oxy)hexyl)oxy)-2-methylpropan-2-yl)carbamate (MPD7c): MPD7c was prepared as a white solid following a similar procedure to MPD1g (yield 43%). ¹H NMR (400 MHz, CDCl₃) 8.30 (s, 1H), 7.68 (dd, J 8.5, 7.3 Hz, 1H), 7.46 (d, J=7.3 Hz, 1H), 7.23 (d, J=8.5 Hz, 1H), 4.97 (dd, J=12.1, 5.4 Hz, 1H), 4.80 (s, 1H), 4.19 (t, J=6.5 Hz, 2H), 3.47 (t, J=6.5 Hz, 2H), 3.34 (s, 2H), 2.95-2.68 (m, 3H), 2.20-2.09 (m, 1H), 1.91 (p, J=6.8 Hz, 2H), 1.69-1.52 (m, 4H), 1.46-1.42 (m, 11H), 1.29 (s, 6H). ¹³C NMR (101 MHz, CDCl₃) δ 171.01, 168.14, 167.09, 165.68, 156.71, 154.93, 136.48, 133.83, 118.94, 117.14, 115.73, 71.35, 69.37, 60.40, 52.73, 49.10, 31.41, 29.40, 28.88, 28.47, 25.82, 25.65, 24.22, 22.64, 21.06.

Synthesis of 4-((6-(2-amino-2-methylpropoxy)hexyl)oxy)-2-(2,6-dioxopiperidin-3-yl)isoindoline-1,3-dione hydrogen chloride (MPD7d): To a solution of MPD7c (600 mg, 1.10 mmol) in dioxane (3 mL) was added HCl in dioxane (2.75 mL, 4M) at room temperature, The reaction mixture was then stirred at RT for 2 h. After completion of reaction by TLC. Remove the solvent by using rotavapor. The solid crude compound directly used for next step. ¹H NMR (400 MHz, CDCl₃) δ 12.29 (s, 1H), 9.28 (s, 3H), 9.00 (dd, J=8.5, 7.2 Hz, 1H), 8.71 (d, J=8.5 Hz, 1H), 8.63 (d, J=7.3 Hz, 1H), 6.27 (dd, J=12.7, 5.4 Hz, 1H), 5.39 (t, J=6.3 Hz, 2H), 4.64 (t, J=6.4 Hz, 2H), 4.53 (s, 2H), 4.08 (ddd, J=16.6, 13.7, 5.4 Hz, 1H), 3.83-3.62 (m, 3H), 3.22 (tdd, J=7.9, 6.8, 3.5 Hz, 1H), 2.96 (p, J=6.5 Hz, 2H), 2.81-2.58 (m, 6H), 2.40 (s, 6H).

Synthesis of (1R,2S,5S)-3-((2S)-2-(3-(1-((6-((2-(2,6-dioxopiperidin-3-yl)-1,3-dioxoisoindolin-4-yl)oxy)hexyl)oxy)-2-methylpropan-2-yl)ureido)-3,3-dimethylbutanoyl)-N—((S)-1-hydroxy-3-((S)-2-oxopyrrolidin-3-yl)propan-2-yl)-6,6-dimethyl-3-azabicyclo[3.1.0]hexane-2-carboxamide (MPD7e): To a solution of this deprotected product MPD7d (150 mg, 0.31 mmol) in anhydrous ACN (5 mL) was added DIPEA (0.167 mL, 0.93 mmol). Then a solution of triphosgene (46 mg, 0.156 mmol) in anhydrous ACN (2 mL) was added to the solution at 0° C. The resulting reaction mixture was stirred at room temperature for 6 h before the addition of a solution of Int. 4 (139 mg, 0.1 mmol) in anhydrous ACN (3 mL). The reaction mixture was then stirred at room temperature overnight. Remove the solvent by rotavapor. Then it was diluted by EtOAc (20 mL), washed with 1 M HCl (20 mL), H₂O (20 mL) and saturated brine solution (20 mL). The organic layer was separated, dried over anhydrous Na₂SO₄, and concentrated in vacuo. The residue was purified with flash chromatography (0-10% MeOH in DCM as eluent) to yield MPD7e as white solid (160 mg, 58%). ¹H NMR (400 MHz, CDCl₃) δ 10.76 (s, 0.5H), 10.58 (s, 0.5H), 7.64 (ddd, J=8.4, 7.3, 5.3 Hz, 1H), 7.42 (dd, J=9.8, 7.2 Hz, 1H), 7.29-7.09 (m, 2H), 6.23 (s, 0.5H), 6.08 (s, 0.5H), 5.61 (dd, J=13.1, 9.8 Hz, 1H), 5.17-5.01 (m, 1H), 4.96 (s, 0.5H), 4.84 (s, 0.5H), 4.35-4.24 (m, 1.5H), 4.23-4.11 (m, 2.5H), 4.10-3.97 (m, 2H), 3.93-3.84 (m, 1H), 3.68-3.57 (m, 2H), 3.54-3.33 (m, 3H), 3.33-3.08 (m, 3H), 3.03-2.73 (m, 3H), 2.59-2.46 (m, 1H), 2.41-2.28 (m, 1H), 2.16-1.95 (m, 2H), 1.91-1.65 (m, 2H), 1.64-1.44 (m, 8H), 1.26-1.09 (m, 5H), 1.06-0.81 (m, 17H).

Synthesis of (1R,2S,5S)-3-((2S)-2-(3-(1-((6-((2-(2,6-dioxopiperidin-3-yl)-1,3-dioxoisoindolin-4-yl)oxy)hexyl)oxy)-2-methylpropan-2-yl)ureido)-3,3-dimethylbutanoyl)-6,6-dimethyl-N—((S)-1-oxo-3-((S)-2-oxopyrrolidin-3-yl)propan-2-yl)-3-azabicyclo[3.1.0]hexane-2-carboxamide (7; MPD7): Compound 7 (MPD7) was prepared as a white solid following a similar procedure to Compound 1 (MPD1) (yield 67%). Analytical data is provided in the table below.

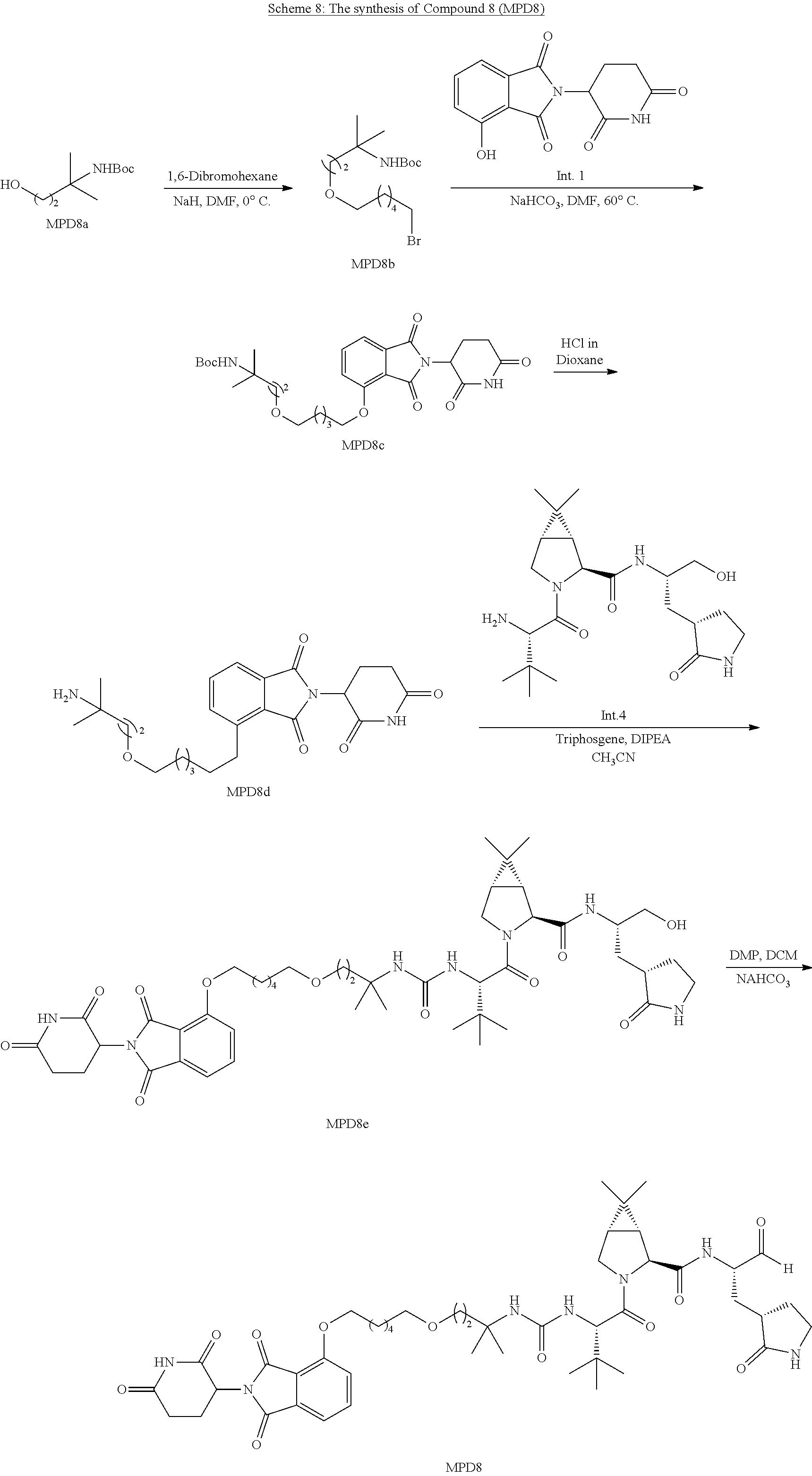

Synthesis of tert-butyl (4-((6-bromohexyl)oxy)-2-methylbutan-2-yl)carbamate (MPD8b): MPD8b was prepared as a white solid following a similar procedure to MPD7b (yield 77%). ¹H NMR (400 MHz, Chloroform-d) δ 5.29 (s, 1H), 3.52 (t, J=6.0 Hz, 2H), 3.40 (td, J=6.6, 2.8 Hz, 4H), 1.86 (p, J=6.8 Hz, 2H), 1.81 (t, J=6.0 Hz, 2H), 1.58 (p, J=6.6 Hz, 2H), 1.48-1.36 (m, 13H), 1.32 (s, 6H).

Synthesis of tert-butyl (4-((6-((2-(2,6-dioxopiperidin-3-yl)-1,3-dioxoisoindolin-4-yl)oxy)hexyl)oxy)-2-methylbutan-2-yl)carbamate (MPD8c): MPD8c was prepared as a white solid following a similar procedure to MPD1g (yield 85%). ¹H NMR (400 MHz, Chloroform-d) δ 8.04 (s, 1H), 7.67 (dd, J=8.5, 7.3 Hz, 1H), 7.45 (d, J=7.2 Hz, 1H), 7.21 (d, J=8.4 Hz, 1H), 5.28 (s, 1H), 5.00-4.89 (m, 1H), 4.18 (t, J=6.5 Hz, 2H), 3.52 (t, J=6.1 Hz, 2H), 3.41 (t, J=6.4 Hz, 2H), 2.93-2.66 (m, 3H), 2.18-2.09 (m, 1H), 1.89 (p, J=6.7 Hz, 2H), 1.81 (t, J=6.1 Hz, 2H), 1.65-1.46 (m, 6H), 1.42 (s, 9H), 1.32 (s, 6H).

Synthesis of (1R,2S,5S)-3-((2S)-2-(3-(4-((6-((2-(2,6-dioxopiperidin-3-yl)-1,3-dioxoisoindolin-4-yl)oxy)hexyl)oxy)-2-methylbutan-2-yl)ureido)-3,3-dimethylbutanoyl)-N—((S)-1-hydroxy-3-((S)-2-oxopyrrolidin-3-yl)propan-2-yl)-6,6-dimethyl-3-azabicyclo[3.1.0]hexane-2-carboxamide (MPD8e): MPD8e was prepared as a white solid following a similar procedure to MPD7d and MPD7e (yield 74%). ¹H NMR (400 MHz, Chloroform-d) δ 10.75 (d, J=13.9 Hz, 1H), 7.66 (ddd, J=8.6, 7.3, 1.5 Hz, 1H), 7.45 (d, J=7.2 Hz, 1H), 7.21 (dd, J=8.5, 3.0 Hz, 1H), 7.13 (dd, J=22.8, 8.4 Hz, 1H), 5.71 (d, J=10.2 Hz, 1H), 5.47 (s, 1H), 5.12 (td, J=12.3, 4.8 Hz, 1H), 4.81 (s, 1H), 4.30 (s, 1H), 4.25-4.15 (m, 2H), 4.11-3.96 (m, 2H), 3.89 (ddd, J=10.5, 5.3, 3.7 Hz, 1H), 3.63 (dddd, J=30.5, 19.0, 11.2, 4.9 Hz, 2H), 3.44-3.16 (m, 6H), 3.05-2.77 (m, 3H), 2.61-2.48 (m, 1H), 2.42-2.30 (m, 1H), 2.16-1.98 (m, 3H), 1.93-1.68 (m, 6H), 1.64-1.41 (m, 9H), 1.24 (d, J=6.2 Hz, 3H), 1.15 (d, J=4.9 Hz, 3H), 1.08 (s, 1H), 1.02 (d, J=7.5 Hz, 3H), 0.97-0.87 (m, 11H).

Synthesis of (1R,2S,5S)-3-((2S)-2-(3-(4-((6-((2-(2,6-dioxopiperidin-3-yl)-1,3-dioxoisoindolin-4-yl)oxy)hexyl)oxy)-2-methylbutan-2-yl)ureido)-3,3-dimethylbutanoyl)-6,6-dimethyl-N—((S)-1-oxo-3-((S)-2-oxopyrrolidin-3-yl)propan-2-yl)-3-azabicyclo[3.1.0]hexane-2-carboxamide (8; MPD8): Compound 8 (MPD8) was prepared as a white solid following a similar procedure to Compound 1 (MPD1) (yield 80%). Analytical data is provided in the table below.

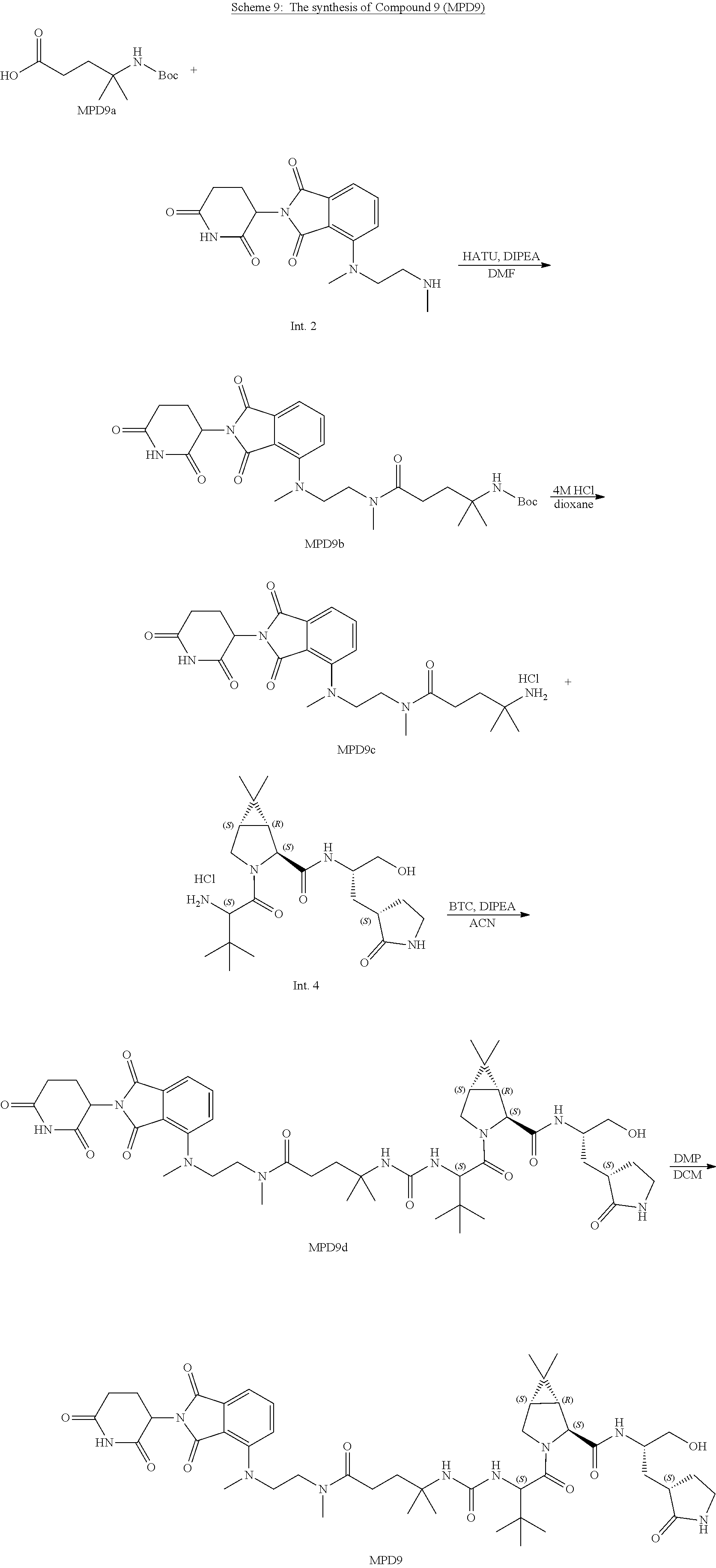

Synthesis of Tert-butyl (5-((2-((2-(2,6-dioxopiperidin-3-yl)-1,3-dioxoisoindolin-4-yl)(methyl)amino)ethyl)(methyl)amino)-2-methyl-5-oxopentan-2-yl)carbamate (MPD9b). MPD9b was prepared as a yellow oil following a similar procedure to MPD5c. (yield 61%). ¹H NMR (400 MHz, Chloroform-d) δ 7.5-7.4 (m, 1H), 7.2 (d, J=6.0 Hz, 1H), 7.1 (d, J=8.6 Hz, 1H), 4.9 (dd, J=12.1, 5.3 Hz, 1H), 3.6 (dddt, J=82.1, 55.6, 13.8, 7.3 Hz, 2H), 3.0 (s, 1H), 2.8 (d, J=20.5 Hz, 10H), 2.3-2.2 (m, 1H), 2.1-2.0 (m, 1H), 1.9 (ddd, J=19.4, 13.0, 9.9 Hz, 2H), 1.7 (t, J=7.8 Hz, 1H), 1.4 (s, 9H), 1.2 (s, 3H), 1.2 (s, 3H). ¹³C NMR (100 MHz, Chloroform-d) δ 177.8, 175.3, 171.7, 168.9, 167.5, 167.1, 149.7, 135.2, 134.2, 123.6, 114.2, 113.6, 77.4, 52.8, 52.0, 49.3, 45.5, 40.1, 38.7, 35.7, 31.5, 29.4, 28.5, 22.7, 20.9.

Synthesis of 4-amino-N-(2-((2-(2,6-dioxopiperidin-3-yl)-1,3-dioxoisoindolin-4-yl)(methyl)amino)ethyl)-N,4-dimethylpentanamide hydrochloride (MPD9c). To a solution of MPD9b in dioxane was added 4.0 M HCl in dioxane. The resulting solution was stirred at RT overnight. After the reaction was completed, remove the solvent in vacuo. The residue was used in the next without further purification.

Synthesis of (1R,2S,5S)-3-((13S)-13-(tert-butyl)-2-(2-(2,6-dioxopiperidin-3-yl)-1,3-dioxoisoindolin-4-yl)-5,9,9-trimethyl-6,11-dioxo-2,5,10,12-tetraazatetradecan-14-oyl)-N—((S)-1-hydroxy-3-((S)-2-oxopyrrolidin-3-yl)propan-2-yl)-6,6-dimethyl-3-azabicyclo[3.1.0]hexane-2-carboxamide (MPD9d). MPD9d was prepared as a white solid following a similar procedure to MPD7e (yield 48%). ¹H NMR (400 MHz, Methanol-d₄) δ 7.7-7.6 (m, 1H), 7.4-7.2 (m, 2H), 5.2-5.1 (m, 1H), 4.3-4.2 (m, 4H), 4.1-4.0 (m, 5H), 3.9 (td, J=6.2, 2.4 Hz, 1H), 3.7 (d, J=9.3 Hz, 2H), 3.6 (d, J=5.7 Hz, 4H), 3.3 (dt, J=3.2, 1.6 Hz, 3H), 3.1 (s, 3H), 2.9 (d, J=1.4 Hz, 3H), 2.8-2.7 (m, 2H), 2.7-2.6 (m, 2H), 2.4-2.3 (m, 2H), 2.3 (td, J=6.6, 3.0 Hz, 1H), 2.2 (dtd, J=12.9, 5.0, 2.1 Hz, 1H), 2.0 (tdd, J=13.7, 6.5, 3.6 Hz, 4H), 1.8-1.7 (m, 3H), 1.6 (ddd, J=6.8, 4.1, 2.4 Hz, 2H), 1.3-1.1 (m, 13H). ¹³C NMR (100 MHz, Methanol-d₄) δ 181.4, 174.5, 173.4, 172.7, 171.8, 170.3, 167.6, 166.9, 158.1, 149.5, 134.9, 134.2, 123.3, 115.7, 113.7, 64.3, 60.8, 57.5, 54.5, 53.5, 51.4, 50.7, 49.1, 49.0, 42.5, 40.1, 39.2, 37.9, 35.3, 34.5, 32.4, 31.0, 30.9, 27.9, 27.7, 26.5, 25.7, 25.2, 22.4, 18.9, 17.4, 16.0, 11.8.

Synthesis of (1R,2S,5S)-3-((13S)-13-(tert-butyl)-2-(2-(2,6-dioxopiperidin-3-yl)-1,3-dioxoisoindolin-4-yl)-5,9,9-trimethyl-6,11-dioxo-2,5,10,12-tetraazatetradecan-14-oyl)-6,6-dimethyl-N—((S)-1-oxo-3-((S)-2-oxopyrrolidin-3-yl)propan-2-yl)-3-azabicyclo[3.1.0]hexane-2-carboxamide (9; MPD9). Compound 9 (MPD9) was prepared as a yellow solid following a similar procedure to Compound 1 (MPD1) (yield 53%). Analytical data is provided in the table below.

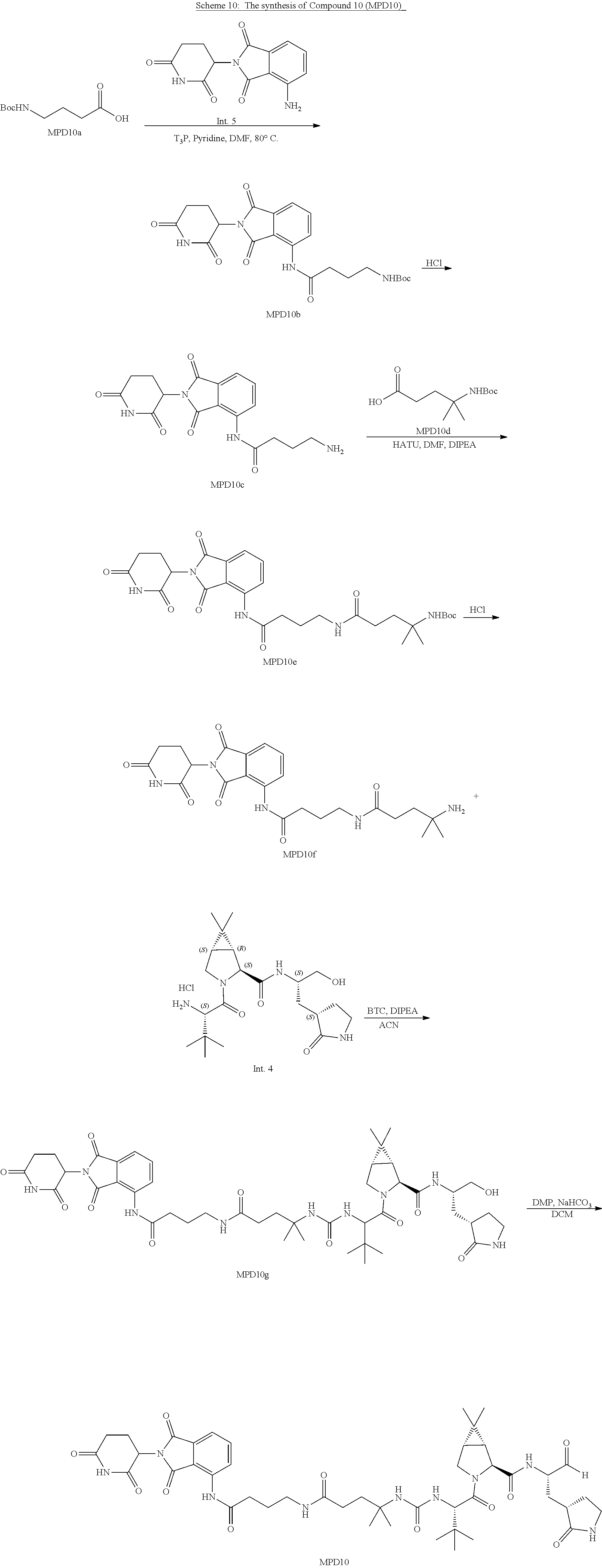

Synthesis of tert-butyl (4-((2-(2,6-dioxopiperidin-3-yl)-1,3-dioxoisoindolin-4-yl)amino)-4-oxobutyl)carbamate (MPD10b). MPD10b was prepared as a white solid following a similar procedure to MPD5e (yield 62%) ¹H NMR (400 MHz, DMSO-d₆) δ 11.15 (s, 1H), 9.70 (s, 1H), 8.52-8.42 (m, 1H), 7.90-7.80 (m, 1H), 7.67-7.56 (m, 1H), 6.87 (t, J=5.8 Hz, 1H), 5.23-5.03 (m, 1H), 3.67-3.59 (m, 1H), 3.00 (q, J=6.5 Hz, 2H), 2.69-2.54 (m, 2H), 2.40 (t, J=8.1 Hz, 1H), 2.14-2.01 (m, 1H), 1.95-1.84 (m, 2H), 1.82-1.64 (m, 2H), 1.45 (s, 6H), 1.38 (s, 8H).

Synthesis of 4-amino-N-(2-(2,6-dioxopiperidin-3-yl)-1,3-dioxoisoindolin-4-yl)butanamide (MPD10c). MPD10c was prepared as a white solid following a similar procedure to MPD9c (yield 77%)¹H NMR (400 MHz, DMSO-d₆) δ 11.18 (s, 1H), 9.86 (s, 1H), 8.39 (d, J=8.3 Hz, 1H), 8.17 (d, J=7.0 Hz, 2H), 7.88 (t, J=7.9 Hz, 1H), 7.66 (d, J=7.3 Hz, 1H), 3.78-3.61 (m, 1H), 3.32 (t, J=7.0 Hz, 1H), 2.71-2.46 (m, 4H), 2.28-1.92 (m, 5H). ¹³C NMR (100 MHz, DMSO-d₆) δ 179.31, 173.57, 173.50, 171.68, 170.62, 170.31, 168.99, 167.78, 167.14, 163.32, 146.90, 136.60, 136.39, 136.06, 131.76, 127.32, 119.19, 118.09, 66.71, 60.51, 49.29, 42.51, 36.56, 33.47, 31.43, 31.27, 30.33, 22.89, 22.36, 20.56.

Synthesis of tert-butyl (5-((4-((2-(2,6-dioxopiperidin-3-yl)-1,3-dioxoisoindolin-4-yl)amino)-4-oxobutyl)amino)-2-methyl-5-oxopentan-2-yl)carbamate (MPD10e). MPD10e was prepared as a yellow oil following a similar procedure to MPD5c. (yield 45%). ¹H NMR (400 MHz, DMSO-d₆) δ 11.15 (s, 1H), 9.70 (s, 1H), 8.85-8.70 (m, 1H), 8.59-8.43 (m, 1H), 7.89-7.73 (m, 2H), 7.67-7.57 (m, 1H), 7.57-7.45 (m, 1H), 7.09-6.93 (m, 1H), 5.24-4.90 (m, 1H), 3.10 (q, J=6.6 Hz, 2H), 2.67-2.52 (m, 2H), 2.30-1.98 (m, 4H), 1.90-1.64 (m, 5H), 1.37 (d, J=1.7 Hz, 11H), 1.30-1.22 (m, 2H), 1.16 (d, J=1.4 Hz, 2H), 1.15 (d, J=0.9 Hz, 6H).

Synthesis of 4-amino-N-(2-(2,6-dioxopiperidin-3-yl)-1,3-dioxoisoindolin-4-yl)butanamide 4-amino-N-(4-((2-(2,6-dioxopiperidin-3-yl)-1,3-dioxoisoindolin-4-yl)amino)-4-oxobutyl)-4-methylpentanamide (MPD10f). MPD10f was prepared as a yellow oil following a similar procedure to MPD9c. (yield 61%). ¹H NMR (400 MHz, DMSO-d6) δ 11.15 (s, 1H), 9.74 (s, 1H), 8.45 (d, J=8.3 Hz, 1H), 8.24-8.02 (m, 4H), 7.84 (dd, J=8.4, 7.3 Hz, 1H), 7.62 (dd, J=7.3, 0.8 Hz, 1H), 7.57-7.38 (m, 1H), 7.15-6.86 (m, 1H), 5.22-5.06 (m, 1H), 3.73-3.62 (m, 1H), 3.54-3.45 (m, 1H), 3.12 (q, J=6.6 Hz, 1H), 2.93-2.85 (m, 1H), 2.63-2.34 (m, 6H), 2.24-1.98 (m, 3H), 1.84-1.71 (m, 3H), 1.25-1.21 (m, 7H).

Synthesis of (1R,2S,5S)-3-((2S)-2-(3-(5-((4-((2-(2,6-dioxopiperidin-3-yl)-1,3-dioxoisoindolin-4-yl)amino)-4-oxobutyl)amino)-2-methyl-5-oxopentan-2-yl)ureido)-3,3-dimethylbutanoyl)-N—((S)-1-hydroxy-3-((S)-2-oxopyrrolidin-3-yl)propan-2-yl)-6,6-dimethyl-3-azabicyclo[3.1.0]hexane-2-carboxamide (MPD10g). MPD10g was prepared as a white solid following a similar procedure to MPD7e (yield 56%). ¹H NMR (400 MHz, Methanol-d4) δ 8.55 (d, J=8.4 Hz, 1H), 7.68 (dd, J=8.5, 7.3 Hz, 1H), 7.49 (d, J=7.3 Hz, 1H), 5.89 (d, J=2.3 Hz, 1H), 5.76 (d, J=9.5 Hz, 1H), 5.04 (dd, J=12.5, 5.4 Hz, 1H), 4.48 (s, 1H), 4.19-4.09 (m, 2H), 3.96-3.82 (m, 2H), 3.69-3.56 (m, 2H), 3.46-3.37 (m, 2H), 3.20-3.08 (m, 5H), 2.83-2.72 (m, 1H), 2.72-2.60 (m, 1H), 2.60-2.48 (m, 1H), 2.45 (t, J=7.4 Hz, 1H), 2.31-2.21 (m, 1H), 2.11-2.01 (m, 2H), 1.86-1.79 (m, 3H), 1.68-1.60 (m, 1H), 1.45 (dd, J=7.7, 5.1 Hz, 1H), 1.37 (t, J=2.7 Hz, 1H), 1.28 (s, 5H), 1.27 (d, J=2.4 Hz, 6H), 1.16-1.11 (m, 3H), 1.10 (s, 2H), 0.92 (d, J=1.6 Hz, 2H), 0.89 (s, 7H), 0.82 (s, 2H).

Synthesis of (1R,2S,5S)-3-((2S)-2-(3-(5-((4-((2-(2,6-dioxopiperidin-3-yl)-1,3-dioxoisoindolin-4-yl)amino)-4-oxobutyl)amino)-2-methyl-5-oxopentan-2-yl)ureido)-3,3-dimethylbutanoyl)-6,6-dimethyl-N—((S)-1-oxo-3-((S)-2-oxopyrrolidin-3-yl)propan-2-yl)-3-azabicyclo[3.1.0]hexane-2-carboxamide (10; MPD10): Compound 10 (MPD10) was prepared as a white solid following a similar procedure to Compound 1 (MPD1) (yield 48%). Analytical data is provided in the table below.

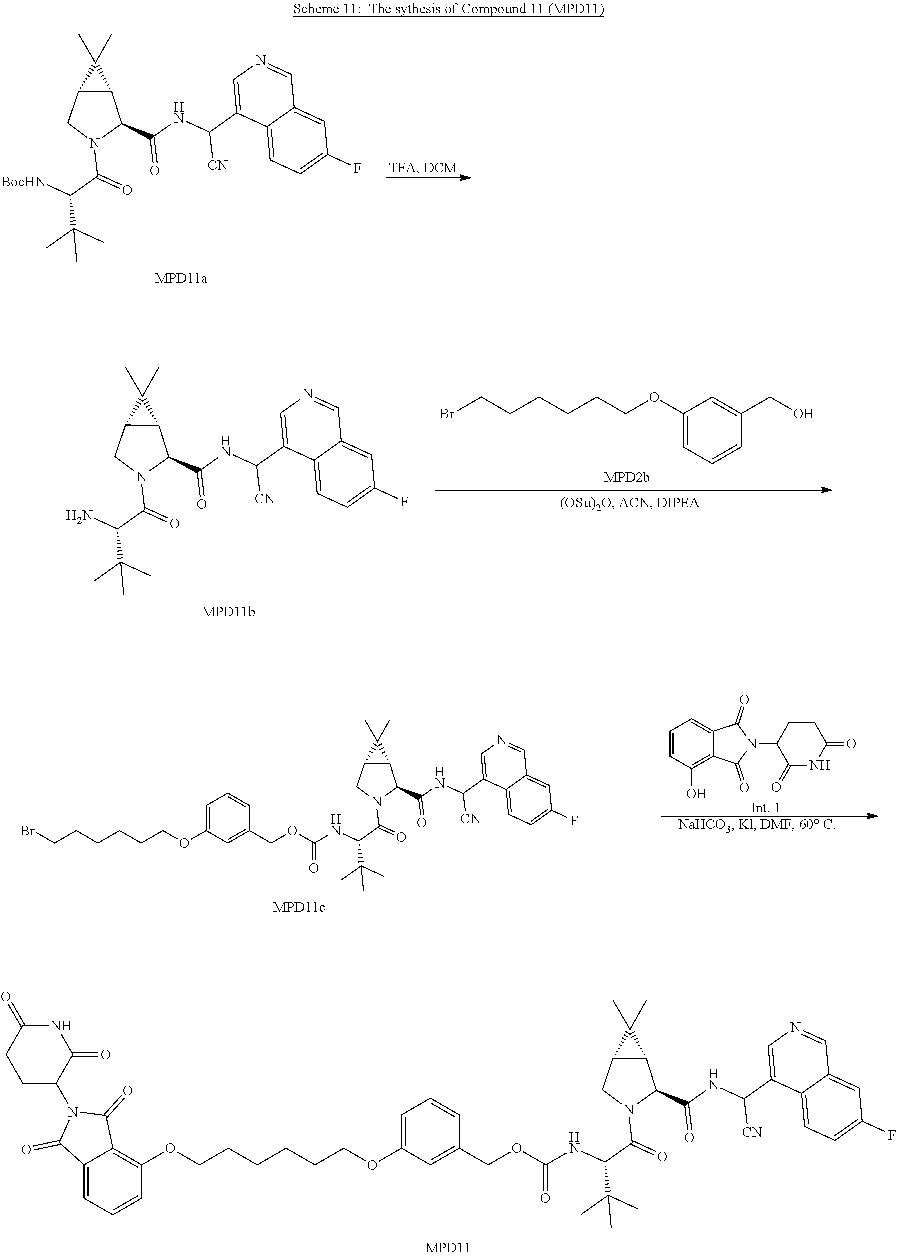 [SMILES: CC(C)(C)OC(=O)N[C@H](C(=O)N1C[C@H]2[C@@H]([C@H]1C(=O)NC(C#N)c1cncc3cc(F)ccc13)C2(C)C)C(C)(C)C ; CC(C)(C)[C@H](N)C(=O)N1C[C@H]2[C@@H]([C@H]1C(=O)NC(C#N)c1cncc3cc(F)ccc13)C2(C)C ; OCc1cccc(OCCCCCCBr)c1 ; CC(C)(C)[C@H](NC(=O)OCc1cccc(OCCCCCCBr)c1)C(=O)N1C[C@H]2[C@@H]([C@H]1C(=O)NC(C#N)c1cncc3cc(F)ccc13)C2(C)C ; O=C1CCC(N2C(=O)c3cccc(O)c3C2=O)C(=O)N1 ; CC(C)(C)[C@H](NC(=O)OCc1cccc(OCCCCCCOc2cccc3c2C(=O)N(C2CCC(=O)NC2=O)C3=O)c1)C(=O)N1C[C@H]2[C@@H]([C@H]1C(=O)NC(C#N)c1cncc3cc(F)ccc13)C2(C)C]

(1R,2S,5S)-3-((S)-2-Amino-3,3-dimethylbutanoyl)-N-(cyano(7-fluoroisoquinolin-4-yl)methyl)-6,6-dimethyl-3-azabicyclo[3.1.0]hexane-2-carboxamide (MPD11b): To a solution of MPD11a (300 mg, 1.10 mmol) in dichloromethane (10 mL) was added TFA (0.6 mL) at room temperature. The reaction mixture was then stirred at RT for 6 h. After completion of reaction by TLC. Te solvent was removed by using rotavapor. The solid crude compound directly used for next step.

3-((6-Bromohexyl)oxy)benzyl ((2S)-1-((1R,2S,5S)-2-((cyano(7-fluoroisoquinolin-4-yl)methyl)carbamoyl)-6,6-dimethyl-3-azabicyclo[3.1.0]hexan-3-yl)-3,3-dimethyl-1-oxobutan-2-yl)carbamate (MPD11c): To a solution of MPD2b (200 mg, 0.775 mmol) in anhydrous ACN (5 mL) was added NMM (236 mg, 2.32 mmol) at 0° C. bis(2,5-dioxopyrrolidin-1-yl) carbonate (278 mg, 1.08 mmol) was slowly added to the solution at the same temperature. The reaction mixture was then allowed to warm up to room temperature and stirred overnight. Then MPD11b (350 mg, crude) was added to reaction and stirred for 2 h. Solvent was removed vacuum and 20 ml Ethyl acetate added to residue. The reaction mixture was then washed by 1 M HCl (2×10 mL). The organic layer was separated, dried over anhydrous Na₂SO₄, and concentrated in vacuo. The residue was purified by flash chromatography (50-100% EtOAc in hexanes as eluent) to yield MPD11c as white solid (50 mg) (1:1 isomers). ¹H NMR (400 MHz, CDCl₃) δ 9.21 (d, J=16.2 Hz, 1H), 8.74 (d, J=3.2 Hz, 1H), 8.10-7.97 (m, 1H), 7.58 (dddd, J=22.8, 17.0, 9.3, 2.6 Hz, 1H), 7.16-7.11 (m, 1H), 6.99-6.91 (m, 1H), 6.87-6.70 (m, 2H), 6.62 (dd, J=25.0, 8.7 Hz, 1H), 5.15 (dd, J=66.5, 10.0 Hz, 1H), 4.92 (dd, J=16.3, 2.8 Hz, 2H), 4.36 (dd, J=16.1, 5.3 Hz, 1H), 4.23-3.56 (m, 5H), 3.35 (tt, J=6.8, 3.0 Hz, 3H), 1.87-1.67 (m, 5H), 1.50-1.34 (m, 5H), 1.00 (d, J=9.2 Hz, 3H), 0.92 (s, 4H), 0.72 (d, J=4.6 Hz, 3H), 0.38 (s, 5H).

3-((6-((2-(2,6-Dioxopiperidin-3-yl)-1,3-dioxoisoindolin-4-yl)oxy)hexyl)oxy)benzyl ((2S)-1-((1R,2S,5S)-2-((cyano(7-fluoroisoquinolin-4-yl)methyl)carbamoyl)-6,6-dimethyl-3-azabicyclo[3.1.0]hexan-3-yl)-3,3-dimethyl-1-oxobutan-2-yl)carbamate (MPD11): To a solution of MPD11c (50 mg, 0.065 mmol) in DMF (2 mL) was added Int.1 (21.5 mg, 0.078 mmol), NaHCO₃ (11 mg, 0.131 mmol), KI (cat). The reaction mixture was then stirred at 60° C. for 24 h. The reaction mixture was then diluted with EtOAc (20 mL), washed with H₂O (20 mL) and saturated brine solution (20 mL). The organic layer was separated, dried over anhydrous Na₂SO₄, and concentrated in vacuo. The residue was purified with flash chromatography (5-10% methanol in EtOAc as eluent) to yield MPD11 as white solid (10 mg).

| Compound | Analytical Data |
|---|---|
| 3-(4-((2-(2,6-dioxopiperidin-3-yl)- | ¹H NMR (400 MHz, DMSO) δ 11.10 (s, 1H), 9.40 (s, 1H), |
| 1,3-dioxoisoindolin-4- | 8.49 (d, J = 7.5 Hz, 1H), 8.08 − 7.89 (m, 1H), 7.81 (dd, J = |
| yl)oxy)butoxy)benzyl ((2S,3R)-3- | 8.5, 7.3 Hz, 1H), 7.63 (s, 1H), 7.57 − 7.48 (m, 1H), 7.44 (d, |
| (tert-butoxy)-1-(((S)-3-cyclohexyl- | J = 7.2 Hz, 1H), 7.25 (t, J = 7.8 Hz, 1H), 6.95 − 6.83 (m, |
| 1-oxo-1-(((S)-1-oxo-3-((S)-2- | 5H), 5.80 − 5.60 (m, 1H), 5.16 − 4.94 (m, 3H), 4.39 (d, J = |
| oxopyrrolidin-3-yl)propan-2- | 7.7 Hz, 1H), 4.20 (q, J = 7.0 Hz, 2H), 4.17 − 3.98 (m, 2H), |
| yl)amino)propan-2-yl)amino)-1- | 3.95 (t, J = 6.5 Hz, 2H), 3.87 (d, J = 5.9 Hz, 1H), 3.25 − |
| oxobutan-2-yl)carbamate (1; | 3.05 (m, 2H), 2.88 (ddd, J = 17.7, 13.9, 5.3 Hz, 1H), 2.58 (d, |
| MPD1) | J = 13.8 Hz, 1H), 2.25 (d, J = 12.1 Hz, 1H), 2.10 (d, J = |
| | 20.2 Hz, 1H), 2.02 (dd, J = 11.7, 6.1 Hz, 1H), 1.96 − 1.28 |
| | (m, 20H), 1.10 (s, 9H), 1.06 − 0.99 (m, 3H), 0.86 (d, J = |
| | 13.5 Hz, 3H). ¹³C NMR (126 MHz, DMSO) δ 200.96, |
| | 178.60, 173.21, 172.94, 172.57, 170.38, 169.94, 167.32, |
| | 165.77, 159.20, 156.52, 156.38, 138.99, 137.48, 133.74, |
| | 129.87, 120.29, 119.97, 116.73, 115.60, 114.18, 114.05, |
| | 74.10, 69.30, 68.03, 67.85, 65.88, 60.16, 56.79, 50.83, |
| | 49.23, 37.69, 34.67, 33.87, 33.49, 32.70, 31.43, 29.67, |
| | 29.18, 29.14, 29.10, 28.88, 28.54, 27.70, 26.49, 26.22, |
| | 26.01, 25.95, 25.91, 25.71, 25.26, 22.52, 22.48, 19.92. ESI- |
| | HRMS m/z calculated for C₄₉H₆₅N₆O₁₃ (M + H⁺): 945.4610; |
| | found: 945.4581. |
| 3-((6-((2-(2,6-dioxopiperidin-3-yl)- | ¹H NMR (400 MHz, DMSO-d₆) δ 11.11 (s, 1H), 9.38 (s, |
| 1,3-dioxoisoindolin-4- | 1H), 8.51 (d, J = 8.0 Hz, 1H), 7.94 (d, J = 7.8 Hz, 1H), 7.80 |
| yl)oxy)hexyl)oxy)benzyl ((2S,3R)- | (t, J = 8.0 Hz, 1H), 7.65 (s, 1H), 7.51 (d, J = 8.8 Hz, 1H), |
| 3-(tert-butoxy)-1-(((S)-3- | 7.44 (d, J = 7.3 Hz, 1H), 7.24 (t, J = 7.9 Hz, 1H), 6.88 (dd, J = |
| cyclohexyl-1-oxo-1-(((S)-1-oxo-3- | 22.4, 9.4 Hz, 4H), 5.15 − 4.92 (m, 3H), 4.44 − 4.26 (m, |
| ((S)-2-oxopyrrolidin-3-yl)propan-2- | 1H), 4.26 − 4.17 (m, 2H), 4.17 − 4.09 (m, 1H), 4.07 − 3.99 |
| yl)amino)propan-2-yl)amino)-1- | (m, 1H), 3.95 (t, J = 6.4 Hz, 2H), 3.91 − 3.80 (m, 1H), 3.24 − |
| oxobutan-2-yl)carbamate (2; | 2.98 (m, 2H), 2.94 − 2.80 (m, 1H), 2.63 − 2.58 (m, 1H), |
| MPD2) | 2.58 − 2.53 (m, 1H), 2.31 − 2.18 (m, 1H), 2.18 − 2.07 (m, |
| | 1H), 2.06 − 1.96 (m, 1H), 1.82 − 1.44 (m, 17H), 1.14 − 1.00 |
| | (m, 16H), 0.96 − 0.81 (m, 4H). ¹³C NMR (126 MHz, |
| | DMSO) δ 200.97, 178.61, 173.21, 172.94, 170.38, 169.94, |
| | 167.32, 165.78, 162.59, 159.20, 156.50, 156.39, 138.98, |
| | 137.49, 133.74, 129.88, 120.30, 119.99, 116.75, 115.62, |
| | 114.16, 74.10, 69.24, 68.03, 67.78, 65.89, 60.16, 56.79, |
| | 50.83, 49.23, 37.69, 33.87, 33.49, 32.70, 31.44, 29.67, |
| | 29.11, 28.84, 28.54, 27.70, 26.49, 26.22, 26.01, 25.65, |
| | 25.52, 22.48, 21.18, 19.91. ESI-HRMS m/z calculated for |
| | C₅₁H₆₉N₆O₁₃ (M + H⁺): 973.4917; found: 973.4903. |
| 3-((8-((2-(2,6-dioxopiperidin-3-yl)- | ¹H NMR (400 MHz, DMSO) δ 11.03 (s, 1H), 9.33 (s, 1H), |
| 1,3-dioxoisoindolin-4- | 8.42 (d, J = 7.6 Hz, 1H), 7.84 (d, J = 7.8 Hz, 1H), 7.75 (dd, J = |
| yl)oxy)octyl)oxy)benzyl ((2S,3R)- | 8.5, 7.3 Hz, 1H), 7.56 (s, 1H), 7.42 (dd, J = 31.9, 7.9 Hz, |
| 3-(tert-butoxy)-1-(((S)-3- | 2H), 7.18 (t, J = 7.9 Hz, 1H), 6.83 (ddd, J = 17.9, 13.3, 8.4 |
| cyclohexyl-1-oxo-1-(((S)-1-oxo-3- | Hz, 4H), 5.72 − 5.50 (m, 1H), 5.06 − 4.87 (m, 3H), 4.31 (q, J = |
| ((S)-2-oxopyrrolidin-3-yl)propan-2- | 7.6 Hz, 1H), 4.22 (s, 2H), 4.07 (ddd, J = 11.6, 7.6, 4.0 Hz, |
| yl)amino)propan-2-yl)amino)-1- | 1H), 4.02 − 3.90 (m, 3H), 3.84 − 3.77 (m, 1H), 3.06 (dt, J = |
| oxobutan-2-yl)carbamate (3; | 25.5, 8.5 Hz, 1H), 2.82 (ddd, J = 16.8, 13.8, 5.3 Hz, 1H), 2.44 |
| MPD3) | (d, J = 1.9 Hz, 2H), 2.16 (dd, J = 14.4, 6.7 Hz, 1H), 2.06 (s, |
| | 1H), 1.99 − 1.92 (m, 1H), 1.88 − 1.76 (m, 5H), 1.73 − 1.14 |
| | (m, 13H), 1.15 − 0.90 (m, 16H), 0.86 − 0.67 (m, 3H). ¹³C |
| | NMR (126 MHz, DMSO) δ 200.97, 179.46, 178.61, 173.21, |
| | 172.94, 172.61, 172.26, 170.38, 169.94, 169.68, 167.32, |
| | 165.81, 159.12, 156.41, 156.38, 138.94, 137.51, 133.73, |
| | 129.89, 120.28, 120.05, 116.77, 115.68, 114.20, 74.11, 74.05, |
| | 69.04, 68.03, 67.55, 65.91, 60.15, 56.79, 52.73, 51.07, 50.83, |
| | 49.24, 37.69, 33.87, 33.76, 33.49, 32.70, 31.44, 29.66, 28.57, |
| | 28.54, 28.14, 27.70, 26.49, 26.23, 26.01, 25.81, 25.67, 22.49, |
| | 19.91, 19.84. ESI-HRMS calculated for C₅₃H₇₁N₆O₁₃ (M − H⁺): |
| | 999.5157; found: 999.5088. ESI-HRMS m/z calculated for |
| | C₅₃H₇₃N₆O₁₃⁺ (M + H)⁺ 1001.5191, found 1001.5220. |
| 3-((6-((2-((2-(2,6-dioxopiperidin-3- | ¹H NMR (400 MHz, Chloroform-d) δ 9.62 − 9.37 (m, 1H), |
| yl)-1,3-dioxoisoindolin-4- | 8.15 − 7.96 (m, 1H), 7.37 (t, J = 8.0 Hz, 2H), 7.23 (d, J = 7.8 |
| yl)(methyl)amino)ethyl)(methyl) | Hz, 1H), 7.14 (d, J = 7.5 Hz, 1H), 6.97 (d, J = 8.2 Hz, 1H), |
| amino)hexyl)oxy)benzyl ((2S,3R)-3- | 6.93 − 6.86 (m, 2H), 6.83 (d, J = 8.3 Hz, 1H), 6.71 (s, 1H), |
| (tert-butoxy)-1-(((S)-3-cyclohexyl- | 5.95 (d, J = 25.4 Hz, 2H), 5.17 − 4.99 (m, 2H), 4.78 (t, J = 8.5 |
| 1-oxo-1-(((S)-1-oxo-3-((S)-2- | Hz, 1H), 4.57 − 4.31 (m, 2H), 4.18 (s, 2H), 3.93 (t, J = 6.5 Hz, |
| oxopyrrolidin-3-yl)propan-2- | 2H), 3.76 (s, 3H), 3.59 − 3.45 (m, 2H), 3.38 − 3.26 (m, 3H), |
| yl)amino)propan-2-yl)amino)-1- | 3.18 (s, 4H), 3.05 − 2.97 (m, 1H), 2.82 (s, 3H), 2.57 − 2.16 |
| oxobutan-2-yl)carbamate (4; | (m, 6H), 2.02 − 1.86 (m, 4H), 1.84 − 1.58 (m, 11H), 1.54 − |
| MPD4) | 1.31 (m, 7H), 1.31 − 1.05 (m, 19H), 1.04 − 0.64 (m, 4H). ESI- |
| | HRMS calculated for C₅₅H₇₈N₈O₁₂Na (M + Na⁺): 1065.5631; |
| | found: 1065.5625. |
| (1-(2-(2-(2-(3-((2-(2,6- | ¹H NMR (400 MHz, CDCl₃) δ 10.14 (d, J = 21.4 Hz, 1H), |
| dioxopiperidin-3-yl)-1,3- | 9.97 (s, 1H), 9.52 (s, 1H), 8.87 (dd, J = 8.5, 2.8 Hz, 1H), |
| dioxoisoindolin-4-yl)amino)-3- | 8.04 (d, J = 7.8 Hz, 1H), 7.80 (d, J = 6.0 Hz, 1H), 7.73 (d, J = |
| oxopropoxy)ethoxy)ethoxy)ethyl)- | 8.5 Hz, 1H), 7.60 − 7.53 (m, 2H), 6.61 (d, J = 14.9 Hz, |
| 1H-1,2,3-triazol-4-yl)methyl | 1H), 5.94 (dd, J = 15.5, 6.9 Hz, 1H), 5.27 (d, J = 12.7 Hz, |
| ((2S,3R)-3-(tert-butoxy)-1-(((S)-3- | 1H), 5.17 (dd, J = 12.8, 6.5 Hz, 1H), 5.06 − 5.02 (m, 1H), |
| cyclohexyl-1-oxo-1-(((S)-1-oxo-3- | 4.54 − 4.50 (m, 2H), 4.49 − 4.44 (m, 1H), 4.38 − 4.34 (m, |
| ((S)-2-oxopyrrolidin-3-yl)propan-2- | 1H), 4.24 − 4.19 (m, 1H), 4.16 − 4.11 (m, 4H), 3.92 − 3.85 |
| yl)amino)propan-2-yl)amino)-1- | (m, 5H), 3.74 − 3.69 (m, 2H), 3.69 − 3.57 (m, 6H), 3.33 (d, J = |
| oxobutan-2-yl)carbamate (5; | 9.2 Hz, 2H), 2.91 − 2.83 (m, 2H), 2.81 − 2.74 (m, 2H), |
| MPD5) | 2.43 − 2.39 (m, 2H), 2.19 − 2.14 (m, 1H), 1.96 − 1.92 (m, |
| | 2H), 1.87 − 1.65 (m, 11H), 1.22 − 1.11 (m, 14H), 0.99 − |
| | 0.90 (m, 2H). ESI-HRMS m/z calculated for C₅₀H₇₁N₁₀O₁₅⁺ |
| | (M + H)⁺ 1051.5056, found 1051.5066. |
| (1-(2-(2-(2,6-dioxopiperidin-3-yl)- | ¹H NMR (400 MHz, CDCl₃) δ 9.43 (s, 1H), 9.19 (d, J = 44.4 |
| 1,3-dioxoisoindolin-4-yl)-5-methyl- | Hz, 1H), 8.02 (s, 1H), 7.74 (d, J = 4.5 Hz, 1H), 7.48 (dt, J = |
| 6-oxo-9,12,15-trioxa-2,5- | 20.9, 7.8 Hz, 1H), 7.41 − 7.28 (m, 1H), 7.08 (d, J = 8.8 Hz, |
| diazaheptadecan-17-yl)-1H-1,2,3- | 1H), 6.40 (d, J = 14.9 Hz, 1H), 5.90 − 5.82 (m, 1H), 5.26 − |
| triazol-4-yl)methyl ((2S,3R)-3-(tert- | 5.13 (m, 1H), 5.12 − 5.03 (m, 1H), 4.96 − 4.85 (m, 1H), 4.58 − |
| butoxy)-1-(((S)-3-cyclohexyl-1- | 4.37 (m, 3H), 4.30 − 4.26 (m, 1H), 4.14 (s, 1H), 4.09 − |
| oxo-1-(((S)-1-oxo-3-((S)-2- | 4.05 (m, 1H), 3.84 − 3.70 (m, 3H), 3.62 − 3.45 (m, 11H), |
| oxopyrrolidin-3-yl)propan-2- | 3.25 (d, J = 8.7 Hz, 2H), 3.06 − 2.94 (m, 3H), 2.90 − 2.66 |
| yl)amino)propan-2-yl)amino)-1- | (m, 5H), 2.62 − 2.52 (m, 1H), 2.35 − 2.22 (m, 3H), 2.05 (d, J = |
| oxobutan-2-yl)carbamate (6; | 9.5 Hz, 1H), 1.95 − 1.49 (m, 12H), 1.32 − 1.26 (m, 1H), |
| MPD6) | 1.18 − 1.00 (m, 18H), 0.93 − 0.80 (m, 2H). ESI-HRMS m/z |
| | calculated for C₅₄H₈₀N₁₁O₁₅⁺ (M + H)⁺ 1122.5791, found |
| | 1122.5812. |
| (1R,2S,5S)-3-((2S)-2-(3-(1-((6-((2- | ¹H NMR (400 MHz, CDCl₃) δ 10.75 (d, J = 36.4 Hz, 0.5H), |
| (2,6-dioxopiperidin-3-yl)-1,3- | 10.48 (s, 0.5H), 9.54 (s, 0.5H), 9.52 (s, 0.5H), 8.23 − 8.13 |
| dioxoisoindolin-4- | (m, 0.5H), 7.96 − 7.80 (m, 0.5H), 7.70 − 7.50 (m, 2H), 7.35 |
| yl)oxy)hexyl)oxy)-2-methylpropan- | (ddd, J = 12.3, 7.2, 1.6 Hz, 1H), 7.13 (d, J = 8.5 Hz, 1H), |
| 2-yl)ureido)-3,3-dimethylbutanoyl)- | 6.54 − 6.25 (m, 1H), 5.61 − 5.51 (m, 1H), 5.09 − 4.78 (m, |
| 6,6-dimethyl-N-((S)-1-oxo-3-((S)- | 2H), 4.50 − 4.41 (m, 1H), 4.32 − 4.19 (m, 2H), 4.17 − 3.91 |
| 2-oxopyrrolidin-3-yl)propan-2-yl)- | (m, 4H), 3.89 − 3.79 (m, 1H), 3.49 − 3.32 (m, 3H), 3.27 − |
| 3-azabicyclo[3.1.0]hexane-2- | 3.06 (m, 3H), 2.93 − 2.67 (m, 3H), 2.50 (pd, J = 9.2, 4.1 Hz, |
| carboxamide (7; MPD7) | 1H), 2.32 − 2.16 (m, 1H), 2.03 − 1.92 (m, 2H), 1.90 − 1.62 |
| | (m, 4H), 1.61 − 1.27 (m, 9H), 1.20 − 1.05 (m, 6H), 1.03 − |
| | 0.90 (m, 6H), 0.86 − 0.78 (m, 9H). ESI-HRMS m/z |
| | calculated for C₄₅H₆₄N₇O₁₁⁺ (M + H)⁺ 878.4619, found |
| | 878.4640. |
| (1R,2S,5S)-3-((2S)-2-(3-(4-((6-((2- | ¹H NMR (400 MHz, Chloroform-d) δ 10.74 (s, 0.41H, |
| (2,6-dioxopiperidin-3-yl)-1,3- | diastereomer 1), 10.66 (s, 0.48H, diastereomer 2), 9.63 − |
| dioxoisoindolin-4- | 9.56 (m, 0.42H, diastereomer 1), 9.56 − 9.50 (m, 0.51H, |
| yl)oxy)hexyl)oxy)-2-methylbutan- | diastereomer 2), 7.69 − 7.62 (m, 1H), 7.62 − 7.49 (m, 1H), |
| 2-yl)ureido)-3,3-dimethylbutanoyl)- | 7.46 − 7.38 (m, 1H), 7.23 − 7.16 (m, 1H), 6.29 − 6.02 (m, |
| 6,6-dimethyl-N-((S)-1-oxo-3-((S)- | 1H), 5.46 (dd, J = 10.1, 6.1 Hz, 1H), 5.13 − 5.01 (m, 1H), |
| 2-oxopyrrolidin-3-yl)propan-2-yl)- | 4.92 − 4.82 (m, 1H), 4.56 − 4.44 (m, 1H), 4.36 − 4.26 (m, |
| 3-azabicyclo[3.1.0]hexane-2- | 2H), 4.22 − 4.12 (m, 2H), 4.01 (t, J = 10.4 Hz, 1H), 3.89 |
| carboxamide (8; MPD8) | (dd, J = 10.4, 5.4 Hz, 1H), 3.42 − 3.32 (m, 4H), 3.32 − 3.14 |
| | (m, 2H), 3.05 − 2.72 (m, 3H), 2.60 − 2.49 (m, 1H), 2.37 − |
| | 2.26 (m, 1H), 2.13 − 1.97 (m, 3H), 1.93 − 1.70 (m, 6H), 1.53 − |
| | 1.40 (m, 4H), 1.28 − 1.10 (m, 8H), 1.07 − 1.00 (m, 3H), |
| | 0.98 − 0.81 (m, 13H). ESI-HRMS m/z calculated for |
| | C₄₆H₆₆N₇O₁₁ (M + H⁺): 892.4815; found: 892.4798. |
| (1R,2S,5S)-3-((13S)-13-(tert- | ¹H NMR (400 MHz, CDCl₃) δ 10.88 (s, 0.5H), 10.56 (s, |
| butyl)-2-(2-(2,6-dioxopiperidin-3- | 0.5H), 9.57 − 9.44 (m, 1H), 7.65 (d, J = 7.3 Hz, 1H), 7.44 |
| yl)-1,3-dioxoisoindolin-4-yl)-5,9,9- | (td, J = 8.7, 7.1 Hz, 1H), 7.13 − 7.00 (m, 1H), 6.30 (d, J = |
| trimethyl-6,11-dioxo-2,5,10,12- | 29.2 Hz, 0.5H), 5.99 (s, 0.5H), 5.61 (d, J = 9.4 Hz, 0.5H), |
| tetraazatetradecan-14-oyl)-6,6- | 5.52 (d, J = 9.8 Hz, 0.5H), 4.96 (ddd, J = 23.6, 12.3, 5.5 Hz, |
| dimethyl-N-((S)-1-oxo-3-((S)-2- | 1H), 4.54 − 4.38 (m, 2H), 4.33 − 4.18 (m, 2H), 4.07 − 3.80 |
| oxopyrrolidin-3-yl)propan-2-yl)-3- | (m, 2H), 3.71 − 3.60 (m, 1H), 3.28 − 3.13 (m, 2H), 3.09 − |
| azabicyclo[3.1.0]hexane-2- | 3.01 (m, 3H), 2.90 − 2.67 (m, 5H), 2.50 (dq, J = 13.7, 4.7 |
| carboxamide (9; MPD9) | Hz, 1H), 2.34 − 2.24 (m, 2H), 2.00 (td, J = 11.6, 6.7 Hz, |
| | 3H), 1.90 − 1.64 (m, 2H), 1.58 − 1.33 (m, 3H), 1.30 − 0.96 |
| | (m, 12H), 0.94 − 0.75 (m, 14H). ESI-HRMS m/z calculated |
| | for C₄₅H₆₄N₉O₁₀⁺ (M + H)⁺ 890.4731, found 890.4763. |
| (1R,2S,5S)-3-((2S)-2-(3-(5-((4-((2- | ¹H NMR (400 MHz, Chloroform-d) δ 10.74 (d, J = 58.9 Hz, |
| (2,6-dioxopiperidin-3-yl)-1,3- | 1H), 9.56 − 9.33 (m, 1H), 8.76 − 8.60 (m, 1H), 7.80 (dd, J = |
| dioxoisoindolin-4-yl)amino)-4- | 7.9, 1.2 Hz, 1H), 7.72 − 7.55 (m, 1H), 7.55 − 7.44 (m, 1H), |
| oxobutyl)amino)-2-methyl-5- | 7.26 (dd, J = 7.5, 1.2 Hz, 1H), 6.99 − 6.90 (m, 1H), 6.33 − |
| oxopentan-2-yl)ureido)-3,3- | 6.06 (m, 1H), 5.14 − 4.96 (m, 1H), 4.27 − 4.14 (m, 1H), 4.10 − |
| dimethylbutanoyl)-6,6-dimethyl-N- | 3.80 (m, 2H), 3.69 (h, J = 6.7 Hz, 1H), 3.64 − 3.48 (m, |
| ((S)-1-oxo-3-((S)-2-oxopyrrolidin- | 1H), 3.44 − 3.30 (m, 1H), 3.30 − 3.11 (m, 2H), 2.87 − 2.70 |
| 3-yl)propan-2-yl)-3- | (m, 2H), 2.64 − 2.38 (m, 2H), 2.38 − 2.20 (m, 1H), 2.20 − |
| azabicyclo[3.1.0]hexane-2- | 2.02 (m, 2H), 1.99 − 1.83 (m, 2H), 1.86 − 1.58 (m, 3H), 1.42 |
| carboxamide (10; MPD10) | (dd, J = 20.5, 7.1 Hz, 6H), 1.25 − 1.15 (m, 7H), 1.12 − 1.02 |
| | (m, 2H), 1.00 − 0.71 (m, 11H). ESI-HRMS m/z calculated |
| | for C₄₅H₆₂N₉O₁₁⁺ (M + H)⁺ 904.4524, found 904.4540. |

### Biological Assay Methods

The following examples are offered to illustrate but not to limit the disclosure. One of skill in the art will recognize that the following assays and methods may be modified by choice of suitable materials and methods.

### Example 1: Materials and Methods

Materials. Yeast extract was purchased from Thermo Fisher Scientific, tryptone from Gibco, Sub3 from Bachem, HEK 293T/17 cells from ATCC, DMEM with GlutaMax from Gibco, FBS from Gibco, polyethyleneimine from Polysciences, and the trypsin-EDTA solution from Gibco. Chemicals were acquired from Sigma Aldrich, Chem Impex, Ambeed, and A21B. Pooled human liver microsome (1910096) was obtained from Xenotech.

M^{Pro} Expression and Purification. The expression plasmid pET28a-His-SUMO-M^{Pro} was constructed in a previous study. This construct was used to transform E. coli BL21(DE3) cells. A single colony grown on an LB plate containing 50 μg/mL kanamycin was picked and grown in 5 mL LB media supplemented with 50 μg/mL kanamycin overnight. This overnight culture was inoculated to 6 L 2YT media with 50 μg/mL kanamycin. Cells were grown to OD₆₀₀ as 0.8. At this point, 1 mM IPTG was added to induce the expression of His-SUMO-M^{Pro}. Induced cells were let grown for 3 h and then harvested by centrifugation at 12,000 rpm, 4° C. for 30 min. Cell pellets were resuspended in 150 mL lysis buffer (20 mM Tris-HCl, 100 mM NaCl, 10 mM imidazole, pH 8.0) and lysed the cells by sonication on ice. The lysate was clarified by centrifugation at 16,000 rpm, 4° C. for 30 min. The supernatant was decanted and mixed with Ni-NTA resins (GenScript). The resins were loaded to a column, the resins were washed with 10 volumes of lysis buffer, and the bound protein was eluted using elution buffer (20 mM Tris-HCl, 100 mM NaCl, 250 mM imidazole, pH 8.0). The buffer of the elute was exchanged to another buffer (20 mM Tris-HCl, 100 mM NaCl, 10 mM imidazole, 1 mM DTT, pH 8.0) using a HiPrep 26/10 desalting column (Cytiva) and the elute was digested using 10 units SUMO protease overnight at 4° C. The digested elute was subjected to Ni-NTA resins in a column to remove His-tagged SUMO protease, His-tagged SUMO tag, and undigested His-SUMO-M^{Pro}. The flow-through was loaded onto a Q-Sepharose column and purified M^{Pro} using FPLC by running a linear gradient from 0 to 500 mM NaCl in a buffer (20 mM Tris-HCl, 1 mM DTT, pH 8.0). Fractions eluted from the Q-Sepharose column were concentrated and loaded onto a HiPrep 16/60 Sephacryl 5-100 HR column and purified using a buffer containing 20 mM Tris-HCl, 100 mM NaCl, 1 mM DTT, and 1 mM EDTA at pH 7.8. The final purified was concentrated and stored in a −80° C. freezer.

In Vitro M^{Pro} Inhibition Potency Characterizations. The assay was conducted using 20 nM M^{Pro} and 10 μM Sub3. All compounds were dissolved in DMSO as 10 mM stock solutions. Sub3 was dissolved in DMSO as a 1 mM stock solution and diluted 100 times in the final assay buffer containing 10 mM NaₓH_{y}PO₄, 10 mM NaCl, 0.5 mM EDTA, and 1.25% DMSO at pH 7.6. M^{Pro} and an inhibitor were incubated in the final assay buffer for 30 min before adding the substrate to initiate the reaction catalyzed by M^{Pro}. The production format was monitored in a fluorescence plate reader with excitation at 336 nm and emission at 490 nm.

Establishment of 293T Cells Stably Expressing M^{Pro}-eGFP. To establish a 293T cell line that stably expresses M^{Pro}-eGFP, lentivirus particles were packaged using the pLVX-M^{Pro}-eGFP plasmid. Briefly, 293T cells were transfected at 90% confluency with three plasmids including pLVX-M^{Pro}-eGFP, pMD2.G, and psPAX2 using 30 mg/mL polyethylenimine. Supernatants were collected at 48 h and 72 h after transfection separately. Lentiviral particles were concentrated and collected from collected supernatant using ultracentrifugation. Fresh 293T cells were then transduced using the collected lentivirus particles. After 48 h of transduction, puromycin was added to the culture media to a final concentration of 2 μg/mL. The puromycin concentration was gradually raised 10 μg/mL in 2 weeks. The final stable cells were maintained in media containing 10 μg/mL puromycin.

Western blot and DC₅₀ (half-maximal degradation concentration) analysis. For protein extraction, 5×105 cells per well were plated onto twelve-well plates and treated with a PROTAC molecule at indicated doses and time duration. Protein extraction and Western blot analysis were performed. In a solution containing 50 mM Tris (pH 7.5), 150 mM NaCl, 5 g/ml aprotinin, 1 g/ml pepstatin, 1% Nonidet P-40, 1 mM EDTA, and 0.25% deoxycholate, cells were lysed. In a cold room, protein lysates were sonicated for two minutes before being centrifuged at maximum speed (14,000 rpm) for 15 minutes. Using the Bradford Reagent (cat. no. 97065-020, VWR), the protein content in the supernatants was measured. After normalizing the protein concentration, the samples were reduced in 4×Laemmli's SDS-sample buffer and desaturated at 95° C. for 10 minutes. Using 10% SDS-PAGE, an equal quantity of protein samples (20 μg each lane) were separated. Subsequent protein signals were produced using Pierce ECL Western blotting substrate (Thermo Scientific; 32106) and visualized using the ChemiDoc MP Imaging System (Bio-Rad). Data were represented as relative band intensities adjusted to equal loading control. Antibody against SARS-CoV-2 M^{Pro} was acquired from Thermo Fisher Scientific (cat. no. PA5-116940). β-actin antibody was purchased from ABCAM (Cat. No. ab124964). Goat Anti-Rabbit IgG H&L (HRP) was acquired from ABCAM as the secondary antibody (Cat. No. ab6721).

Dose-response curves for the tested drugs in M^{Pro}-eGFP 293T stable cells. Various dilutions of the drugs were applied to the 80% confluent cell monolayers and assayed after 48 h to determine the DC₅₀ value. β-actin was used as a loading control in all immunoblot analyses. The normalized protein content in the immunoblots is presented as mean values±s.e.m. (n=3 biologically independent experiments). Nonlinear regression analysis of GraphPad Prism software (version 8.0) was used to calculate DC₅₀ by plotting log compounds concentration versus normalized response (variable compounds).

CRBN knockout by CRISPR/Cas9 genomic editing. To deplete CRBN, CRISPR/Cas9 KO Plasmids, consisting of CRBN-specific 20 nt guide RNA sequences derived from the GeCKO (v2) library, were purchased from Santa Cruz (sc-425519). Briefly, In a 6-well tissue culture plate seed 1.5×105-2.5×105 cells in 3 ml of antibiotic-free standard growth medium per well, 24 hours prior to transfection. Grow cells to a 40-80% confluency. Initial cell seeding and cell confluency after 24 hours are determined based on the rate of cell growth of the cells used for transfection. Healthy and subconfluent cells are required for successful knockout.

Viruses. SARS-CoV-2 strains WA.1 (2019-nCoV/USA_WA1/2020) and BA.1 (GISAID EPI_ISL_6640916) were recovered from VeroE6-TMPRSS2 cells by using the established SARS-CoV-2 reverse genetic system (Reference: Cell Host Microbe. 2020 May 13; 27(5):841-848.e3.; Emerg Microbes Infect. 2023 December; 12(1):e2161422.). XBB.1.5/omicron strain 21554-25363 (GenBank OX397341.1) was obtained through the World Reference Center for Emerging Viruses and Arboviruses (WRCEVA) at UTMB. A highly attenuated WA.1-derived Δ3678 mGFP SARS-CoV-2 was used as a backbone to incorporate the NSP5 E166A mutation (reference: Viruses. 2023 September; 15(9): 1855.). SARS-CoV-2 infections were performed at the BSL-3 facility at UTMB by personnel equipped with powered air-purifying respirators.

Live virus antiviral testing against SARS-CoV-2 delta variant. SARS-CoV-2 delta variant hCoV-19/USA/MD-HP05647/2021 (BEI Resources, NR-55672) was propagated in A549-hACE2 cells (BEI Resources NR-53522) for antiviral testing, at 37° C. in an air-jacketed incubator, with 5% CO₂ and >90% relative humidity, under BSL-3 conditions at the Texas A&M Global Health Research Complex. A low-dose, multi-step growth protocol was used for live virus EC₅₀ assays. Briefly, A549-hACE2 cells were cultured in DMEM supplemented with 10% fetal bovine serum overnight. Approximately 5×10⁴ A549-hACE2 cells were inoculated by adding 103 infectious units of SARS-CoV-2, as determined by tissue culture infectious dose 50% (TCID₅₀) assay, and incubated at 37° C. for 1 hour. Cells were then aspirated and rinsed three times with room temperature phosphate buffered saline, to remove residual inoculum, before replacing DMEM with 10% FBS. Serial three-fold dilutions of candidate antivirals were made in DMEM with 10% FBS and added to three replicate wells per treatment condition. Infected, treated cells were then cultured for 72 h at 37° C., 5% CO₂. At 48 h, and 72 h after inoculation, 50 microliters of tissue culture medium were removed from each sample, for quantitative SARS-CoV-2 reverse transcriptase quantitative PCR (RT-qPCR). After 72 h, cell culture supernatant was removed by aspiration, cells were fixed in 10% formalin, 1×phosphate buffered saline for at least 30 min, then stained with crystal violet in order to qualitatively assess cytopathic effect. EC₅₀ values were calculated from the slope and intercept of log-transformed RT-qPCR results, at the point where the linear portion of the transformed dose-response curve showed 50% reduced growth compared to infected, untreated controls. Samples were processed and RT-qPCR was performed. Samples were diluted 1:1 in 2×Tris-borate-EDTA [TBE] containing 1% Tween-20 and heated at 95° C. for 15 min to lyse and inactivate virions. RT-qPCR screening was performed using the CDC N1 oligonucleotide pair/FAM probe (CDC N1-F, 5′-GACCCCAAAATCAGCGAAAT-3′ (SEQ ID NO.: 1); CDC N1-R, 5′-TCTGGTTACTGCCAGTTGAATCTG-3′ (SEQ ID NO.: 2); and Probe CDC N1, 5′ FAM-ACCCCGCATTACGTTTGGTGGACC-BHQ1 3′ (SEQ ID NO.: 3)) and the Luna Universal Probe one-step RT-qPCR kit (catalog no. E3006; New England Biolabs). A 20-μL RT-qPCR mixture contained 7 μL of sample, 0.8 μL each of forward and reverse oligonucleotides (10 μM), 0.4 μL of probe (10 μM), and 11 μL of NEB Luna one-step RT-qPCR 2× master mix. Samples were incubated at 55° C. for 10 min for cDNA synthesis, followed by 95° C. for 1 min (1 cycle), then 41 cycles of 95° C. for 10 s and 60° C. for 30 s. Genome copies were quantitated relative to quantitative PCR control RNA from heat-inactivated SARS-Related Coronavirus 2, Isolate USA-WA1/2020 (BEI Resources, NR-52347.

Antiviral assays against authentic SARS-CoV-2. A549-hACE2 cells (1.2×10⁴) were seeded in each well of a flat-bottom 96-well plate (NUNC) and incubated at 37° C., 5% CO₂. The next day, cells were infected with WA.1 (MOI 0.1), BA.1 (MOI 0.3), or XBB.1.5 (MOI 0.1) at 37° C. for 1 h. Two-fold serial dilutions of compounds were prepared in dimethyl sulfoxide (DMSO). The compounds were further diluted 200-fold in a culture medium containing 2% FBS. After infection, the inoculum was replaced by 100 μL of fresh medium assay containing diluted compound. At 48 h post-infection, 80 μL per well of supernatants were collected and mixed with 400 μL Trizol LS (ThermoFisher Scientific). RNAs were extracted by the Direct-zol™ RNA Miniprep Plus kit (Zymo Research) and eluted in 50 μL RNase-free water. RT-qPCR was performed to quantify the viral RNA copies. Briefly, 2 μL of RNA samples were used in a 20-μL reaction system with iTaq SYBR Green One-Step Kit (Bio-Rad) and a pair of primers (CoV19-N2-F: TTACAAACATTGGCCGCAAA (SEQ ID NO.: 4); and CoV19-N2-R: GCGCGACATTCCGAAGAA (SEQ ID NO.: 5)) targeting the N-gene. A standard RT-qPCR was performed on the Applied Biosystems QuantStudio 7 system (ThermoFisher Scientific) following the manufacturers' instructions. The relative RNA levels were calculated by normalizing the cycle threshold (CT) values from DMSO controls (DMSO control was treated as 100%). Two to three independent experiments were performed.

Antiviral assays against Δ3678 mGFP SARS-CoV-2. A549-hACE2 cells (1.0×10⁴) were seeded in each well of a flat-bottom 96-well plate (NUNC) and incubated at 37° C., 5% CO₂. The next day, 2-fold serial dilutions of compounds were prepared in dimethyl sulfoxide (DMSO). The compounds were further diluted 100-fold in a 2% FBS culture medium containing diluted Δ3678 mGFP SARS-CoV-2s (MOI 0.2). 50 μL of compound-viruses mixture was added to each well for infection. At 48 h post-infection, 25 μL of a Hoechst 33342 solution (400-fold diluted in PBS; ThermoFisher Scientific) was added to each well to counterstain the cell nuclei. After 20 min of incubation at 37° C., cells were scanned using the CellInsight CX5 high-content screening platform (ThermoFisher Scientific) with predefined threshold parameters obtained using non-infected and infected cells. The positive cells in each well were counted and normalized to the number of total cells, resulting in the infection rate. The infection rate from each well was finally normalized to the DMSO-treated controls to calculate the relative infectivities. The relative infectivity versus the compound concentration (log 10 values) was plotted using Prism 10. A nonlinear regression method with the log (inhibitor) vs. response-variable slope (four parameters) model (bottom and top parameters were constrained to 0 and 100, respectively) was used to determine the compound concentration that inhibits 50% of mGFP Δ3678 infection (defined as EC₅₀) in Prism 10. Three independent experiments were performed.

Cytotoxicity Assay of the PROTAC degraders. To assess the half-maximal cytotoxic concentration (CC₅₀), stock solutions of the tested PROTAC compounds were dissolved in DMSO (10 mM) and diluted further to the working solutions with DMEM. HEK293T cells were seeded in 96 well-plates and incubated at 37° C. and 5% CO₂ for 24 h. The cells were then treated with different concentrations (200 mM, 100 mM, 50 mM, 25 mM, 12.5 mM, 6.25 mM, 3.125 mM, 1.5625 mM, 0.78125 mM, 0 mM) of the tested compounds in triplicates for 48 h. Cell viability was assessed by MTT assay to determine CC₅₀. 20 mL MTT (5 mg/mL) was incubated per well for 4 h and then after removing supernatant, 200 mL DMSO was added per well. The absorbance was recorded at 490 nm to determine the CC₅₀. The CC₅₀ values were obtained by plotting the normalization % cell viability versus log₁₀ sample concentration.

In Vitro Metabolic Stability in Human Liver Microsomes. The metabolic stability measurements were modified as described below. The metabolic stability profile of the inhibitor, including CL_{int, pred} and in vitro t_{1/2} was determined by the estimation of the remaining compound concentration after incubation with human liver microsome, NADPH (cofactor), EDTA, and MgCl₂ in a 0.1 M phosphate buffer (pH 7.4). 5 μM of each inhibitor was preincubated with 40 μL of human liver microsome (0.5 mg/mL) in 0.1 M phosphate buffer (pH 7.4) at 37° C. for 5 min to set optimal a condition for metabolic reactions. After pre incubation, NADPH (5 mM, 10 μL) or 0.1 M PB (10 μL) was added to initiate metabolic reaction at 37° C. The reactions were conducted in triplicate. At 0, 5, 15, 30, 45, 60 min, 200 μL acetonitrile (with internal standard Diclofenac, 10 μg/mL) was added in order to quench the reaction. The samples were subjected to centrifugation at 4° C. for 20 min at 4000 rpm. Then 50 μL of clear supernatants were analyzed by HPLC-MS/MS. The percentage of test compound remaining was determined by following formula: % remaining=(Area at tₓ/Average area at to)×100. The half-life (t_{1/2}) was calculated using the slope (k) of the log-linear regression from the % remaining parent compound versus time (min) relationship: t_{1/2} (min)=−ln 2/k. CL_{int, pred} (mL/min/kg) was calculated through following formula CL_{int, pred}=(0.693/t_{1/2})×(1/(microsomal protein concentration (0.5 mg/mL))×Scaling Factor (1254.16 for human liver microsome).

### Example 2

### Design of M^{Pro} PROTAC Degraders (MPDs) Based on Reversible Covalent Inhibitors MPI8 and MPI29

M^{Pro}, a protein consisting of 306 amino acids, features an active site comprising four small pockets responsible for binding to the P1, P2, P4, and P1′-3′ residues within a protein substrate. Crucially, M^{Pro} specifically recognizes a protein substrate containing a strictly P1 glutamine residue. Two active site residues Cys145 and His41 form a catalytic dyad. An effective strategy for developing M^{Pro} inhibitors involves the conversion of the hydrolytic peptide bond within a substrate into an electrophilic warhead (e.g. aldehyde) to facilitates the formation of a covalent adduct with the catalytic Cys145 of M^{Pro}. In 2020, building upon insights from prior medicinal chemistry studies on SARS-CoV M^{Pro} inhibitors, the design and synthesis of new reversible covalent inhibitors for SARS-CoV-2 M^{Pro} was explored by: (i) employing a more potent β-(S-2-oxopyrrolidin-3-yl)-alanine as the fixed P1 residue and enhancing binding due to reduced entropy loss when M^{Pro} engages with the more rigid lactam; (ii) utilizing aldehyde group as an electrophilic warhead to form a reversible covalent bond with active site Cys145; (iii) optimizing the P2, P3 and P4 positions for improved potency. Their enzymatic inhibition and antiviral potency were characterized, and determined the crystal structures of M^{Pro} bound with these inhibitors. Among these inhibitors, MPI8 (as shown in Table A) emerged with the highest cellular potency against M^{Pro} and highest antiviral activity against SARS-CoV-2. X-ray crystallography analysis of the M^{Pro}-MPI8 complex (PDB ID: 7JQ5) revealed that MPI8 fits precisely in the P1- and P2-binding pockets at the M^{Pro} active site. Strong van der Waals interactions at the P1- and P2-binding pockets, a number of hydrogen bonds with active-site residues, and the covalent interaction with C145 to form a hemithioacetal adduct contribute to high affinity of MPI8 binding to M^{Pro}. The N-terminal phenyl group of MPI8 and other inhibitors are not well defined in the crystal structures, indicating an unfitting size or relatively loosely bound pattern within P4-binding pocket.

TABLE A

| Name | Structure |
|---|---|
| MPI8 | 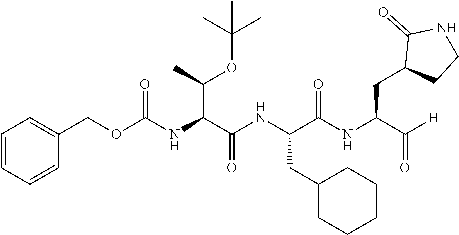 [SMILES: C[C@@H](OC(C)(C)C)[C@H](NC(=O)OCc1ccccc1)C(=O)N[C@@H](CC1CCCCC1)C(=O)N[C@H](C=O)C[C@@H]1CCNC1=O] |
| MPI29 | 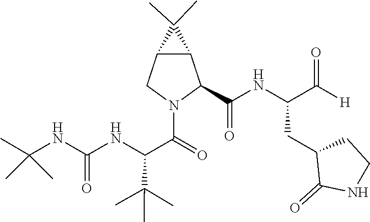 [SMILES: CC(C)(C)NC(=O)N[C@H](C(=O)N1C[C@H]2[C@@H]([C@H]1C(=O)N[C@H](C=O)C[C@@H]1CCNC1=O)C2(C)C)C(C)(C)C] |
| Sub3¹ | 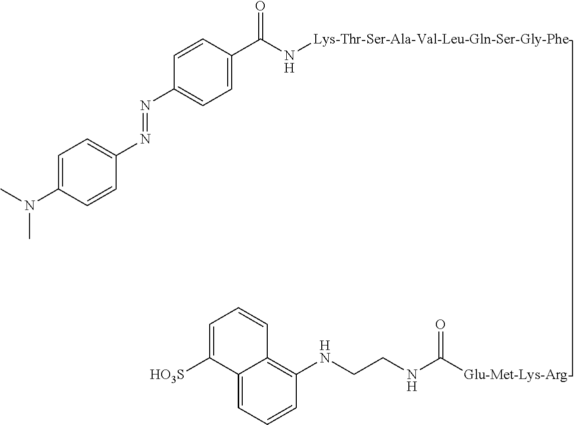 |

¹See SEQ ID NO.: 5

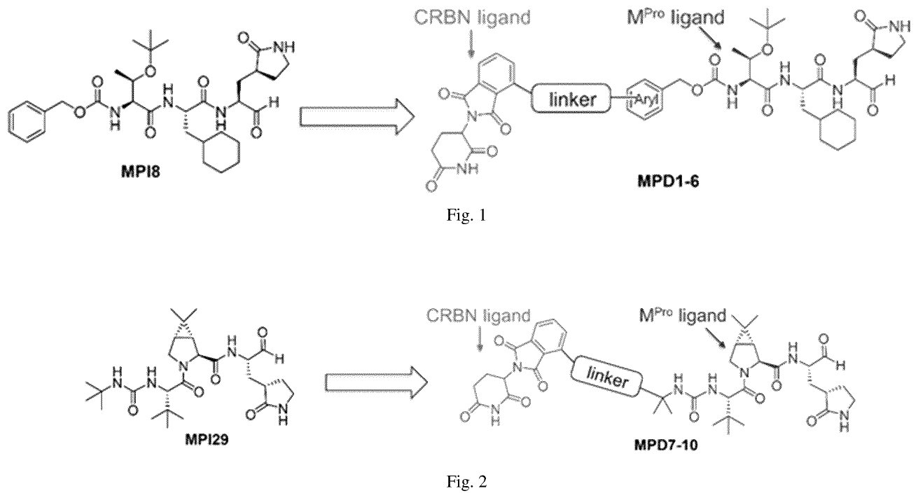

Boceprevir has been explored as a repurposed drug for COVID-19. It serves as an M^{Pro} inhibitor and contains an α-ketoamide warhead, a P1 β-cyclobutylalanyl moiety, a P2 dimethylcyclopropylproline, a P3 tert-butylglycine, and an N-terminal tert-butylcarbamide. MPI29 (as shown in Table A) was designed and synthesized by replacing the P1 site of Boceprevir with a 5-oxopyrrolidine-containing residue and altering the warhead to an aldehyde, resulting in an incredibly potent enzymatic inhibitor (IC₅₀=9.3 nM). In the M^{Pro}-MPI29 complex (PDB ID: 7S6W), MPI29 forms a covalent adduct with M^{Pro} C145 and multiple hydrogen bonds with M^{Pro}. Notably, the amide of the lactam at P1 side chain of MPI29 engages in three hydrogen bonds with M^{Pro} residues including F140, H163, and E166. Additionally, two hydrogen bonds are generated between two backbone amides of MPI29 and M^{Pro} residues involving H164 and E166. The P4 N-terminal urea cap of MPI29 employs its two nitrogen atoms to form hydrogen bonds with the backbone oxygen of M^{Pro} E166 (FIG. 1B). This represents a new strategy to maintain the catalytic nature of PROTACs while retaining the advantages of covalent PROTACs, such as improved potency, selectivity, and reduced resistance, and with the added advantage of reversibility that could decrease the potential toxicity related to permanent off-target protein labelling. Hence, reversible covalent M^{Pro} ligands MPI8 and MPI29 were chosen for the development of covalent PROTAC degraders for M^{Pro} by taking the advantages of the strengths of reversible covalent inhibitors and event-driven PROTAC technology.

The crystal structures of M^{Pro}-MPI8 complex (PDB: 7JQ5) and M^{Pro}-MPI29 complex (PDB: 7S6W) provided a structural basis to rationally choose a link site for the design of PROTAC degraders. Ubiquitin E3 ligase cereblon (CRBN) ligands were explored as the E3 ligase binders. Drawing insights from crystal structures of M^{Pro}-MPI8 complex (PDB: 7JQ5), it became apparent that the N-terminal phenyl moiety of MPI8 exhibited a less defined binding pattern within the P4-binding pocket. These observations guided the design of a series of M^{Pro} degraders, achieved through the incorporation of a ligand for the CRBN E3 ligase using a diverse array of linkers (FIG. 1): (i) modification of the N-terminal phenyl group of MPI8 with different linkers; and (ii) substitution of N-terminal phenyl group of MPI8 with a triazole via Cu-catalyzed azide-alkyne cycloaddition with different linkers. Building on crystal structures of M^{Pro}-MPI29 complex (PDB: 7S6W), the N-terminal tert-butyl moiety of MPI29 at the P4 site was identified as a suitable linker site for introducing CRBN ligand without interrupting its binding with M^{Pro} (FIG. 2).

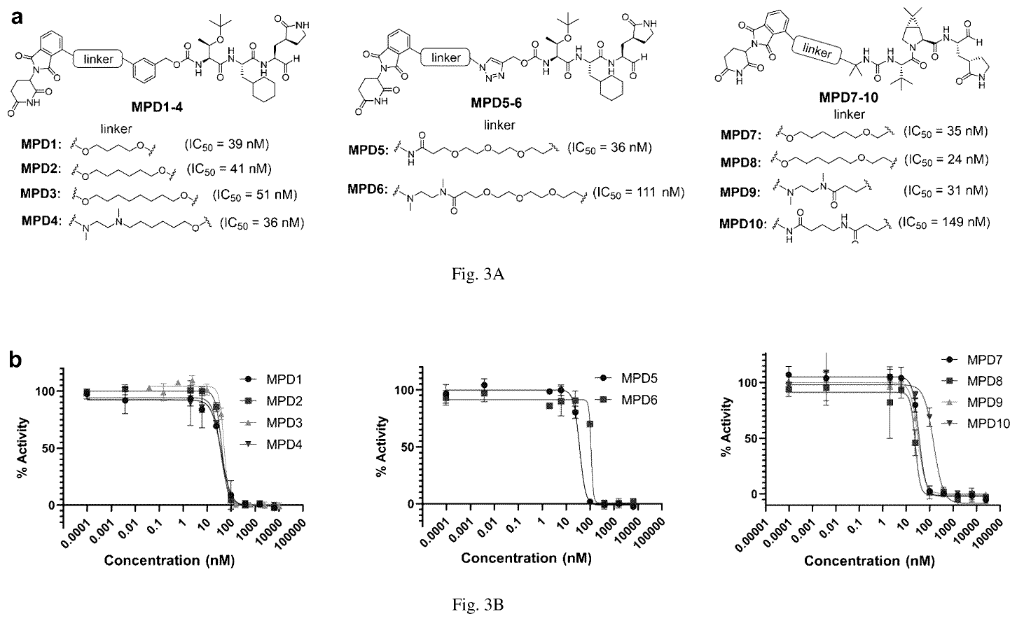

A series of bifunctional small-molecule PROTACs, namely MPD1-MPD6 based on MPI8 and MPD7-MPD10 based on MPI29, were synthesized as shown in FIG. 3A. The CRBN-binding moieties of MPD1-MPD3 and MPD4 were derived from thalidomide-4-OH and pomalidomide, respectively, two widely used CRBN binders for the PROTAC development. For MPD5-MPD6, the N-terminal phenyl group of MPI8 was replaced with a triazole and introduced pomalidomide CRBN ligand with different linkers via Cu-catalyzed azide-alkyne cycloaddition. For MPI29-based PROTACs MPD7-10, one of methyl groups of the N-terminal tert-butyl moiety of MPI29 was modified to introduce thalidomide-4-OH and pomalidomide CRBN ligands (FIG. 3A). To assess the inhibitory potency of MPD1-MPD10, an established protocol that uses Sub3 (Dabcyl-KTSAVLQSGFRKME-Edans) (as shown in Table A; (SEQ ID NO: 5)), a fluorogenic peptide substrate of M^{Pro} was used. In this assay, M^{Pro} was preincubated with a PROTAC molecule for 30 min before Sub3 was added and the fluorescent product formation (Ex: 336 nm/Em: 490 nm) was recorded in a fluorescence plate reader. As illustrated in FIG. 3B, all MPD1-MPD10 retained sub-micromolar IC₅₀ values for the inhibition of SARS-CoV-2 M^{Pro}, which indicates that the structure-based design of linker sites works well as there were no obvious interferences with MPDs binding to M^{Pro}.

### Example 3: MPD2 is a Potent M^{Pro} Degrader

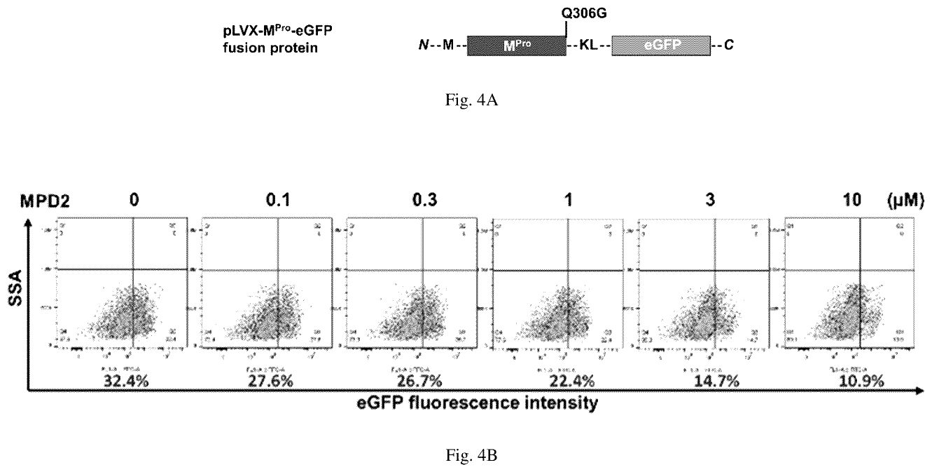

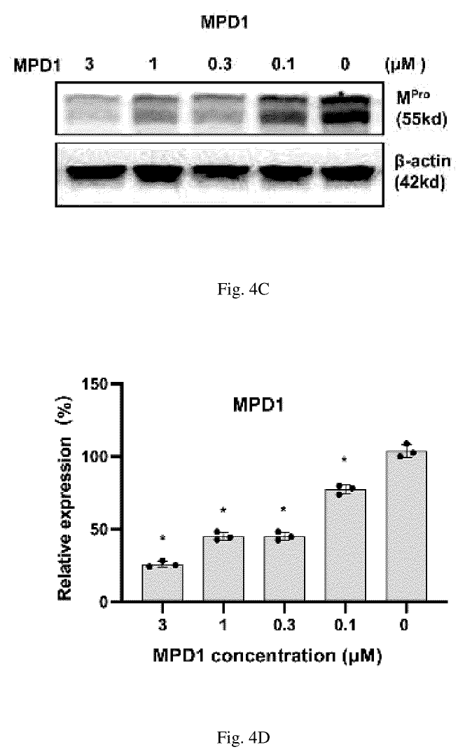

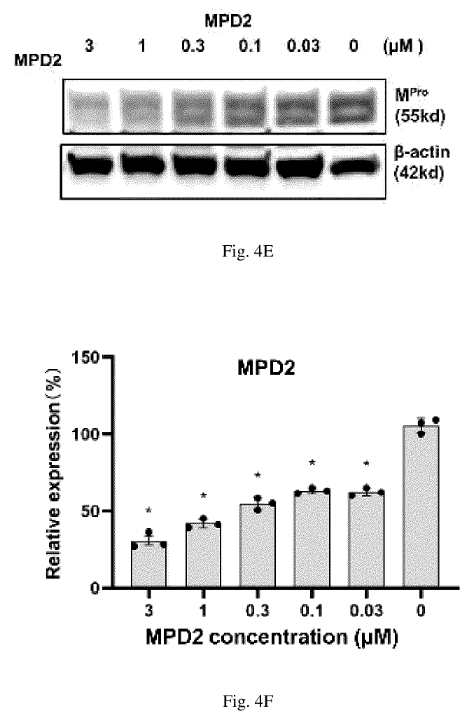

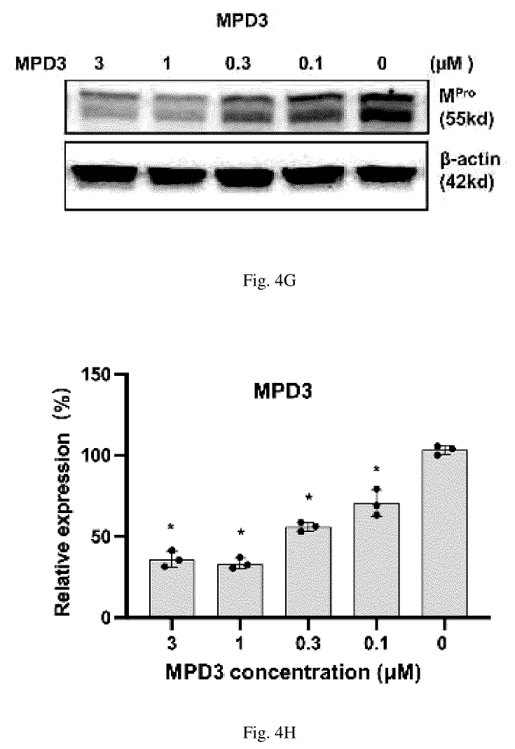

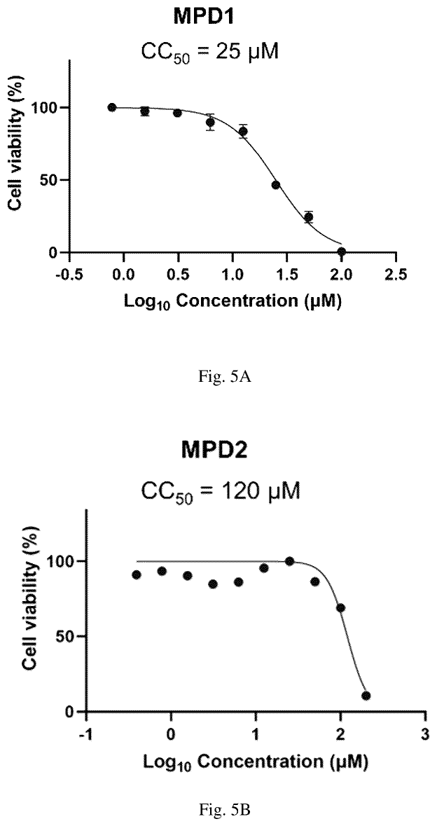

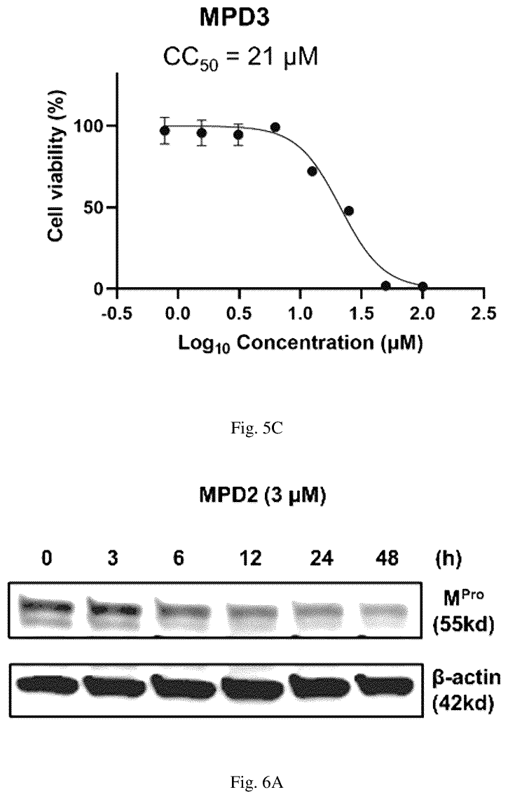

To characterize the degradation of M^{Pro} by PROTAC degraders MPD1-MPD10, a cellular system with a stable expression of M^{Pro} was established. For this purpose, a lentivirus-based system capable of expressing an M^{Pro}-eGFP fusion protein was constructed (FIG. 4A). This system allowed the generation of 293T cells that stably express M^{Pro}-eGFP. M^{Pro} natively undergoes cleavage at its N- and C-terminus and requires a free N-terminal end for activation. To maintain the activity of M^{Pro}, an N-terminal MAVLQ (SEQ ID NO.: 6) self-cleavage tag was introduced to the fusion protein. Additionally, to prevent the cleavage of the C-terminal eGFP, the C-terminal residue Q306 of M^{Pro} was mutated to Gly. Importantly, the transfection of 293T cells with pLVX-M^{Pro}-eGFP showed much less toxicity after multiple rounds of proliferation than an alternative construct. This helped to generate stable cell lines with high levels of eGFP fluorescence for evaluating M^{Pro} PROTACs. The flow cytometry results confirmed the expression of the fusion protein, displaying strong fluorescence (FIG. 4B). This was further corroborated with Western blot analysis to determine half-maximal degradation concentration (DC₅₀) using an anti-M^{Pro} antibody. Among these M^{Pro} PROTACs, MPD1, MPD2, and MPD3 demonstrated high potency in inducing M^{Pro} degradation in M^{Pro}-eGFP stable cell lines. Their DC₅₀ values were 419 nM, 296 nM, 431 nM, respectively (FIGS. 4C-4H). Next, the cytotoxicity of MPD1-MPD3 was evaluated using 293T cells and an MTT assay. The determined CC₅₀ values for MPD1, MPD2 and MPD3 were 25 μM, 120 μM, and 21 μM, respectively (FIGS. 5A-5C). The cytotoxicity curves for these three compounds are presented in FIGS. 5A-5C. Notably, MPD2 exhibited significantly lower toxicity even when compared to the parental MPI8 (CC₅₀=70 μM). Consequently, MPD2 was selected for further evaluation.

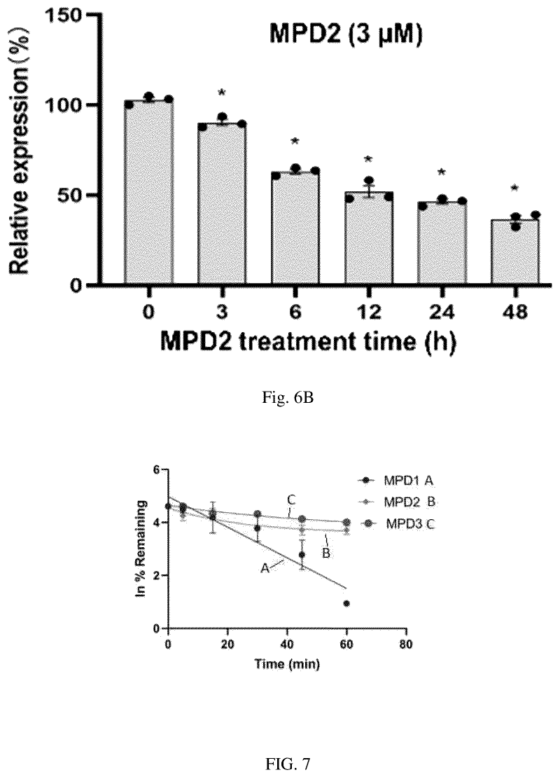

The induction of M^{Pro} degradation by MPD2 in M^{Pro}-eGFP 293T stable cells was rapid and long-lasting. As illustrated in FIGS. 6A and 6B, significant degradation was already observed after 6 h of incubation with 3 μM MPD2, and nearly maximal degradation was achieved after 12 h of incubation. To evaluate the in vitro metabolic stability of MPD2, an analysis using human liver microsomes was conducted. The determined clearance (CLᵢₙₜ) value for MPD2 was 36.2 mL/min/kg, resulting in a half-life (t_{1/2}) of 48 minutes (FIG. 7, and Table B).

TABLE B

| | | Intrinsic clearance |
|---|---|---|
| Sample ID | Half life (min) | (mL/min/kg) |
| MPD1 | 11.7 ± 1.04 | 149.4 ± 12.7 |
| MPD2 | 48.05 ± 3.22 | 36.28 ± 2.51 |
| MPD3 | 70.2 ± 1.5 | 24.7 ± 0.5 |

### Example 4: Mechanism of M^{Pro} Degradation by MPD2

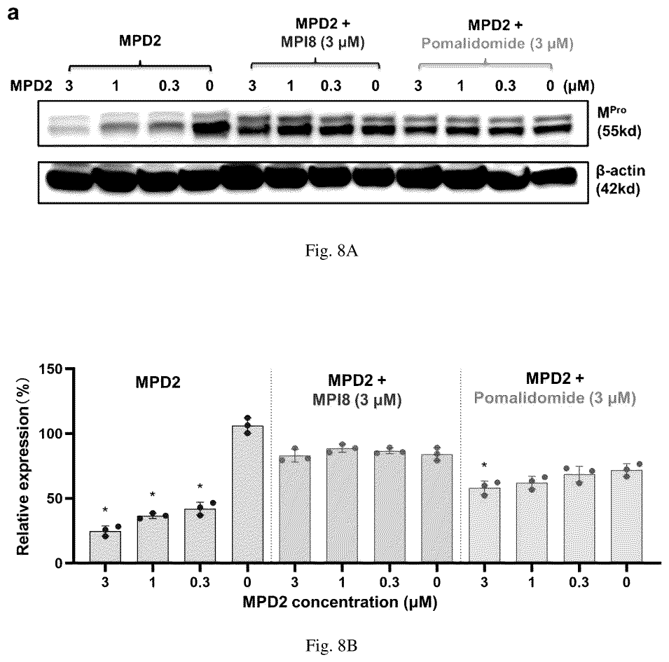

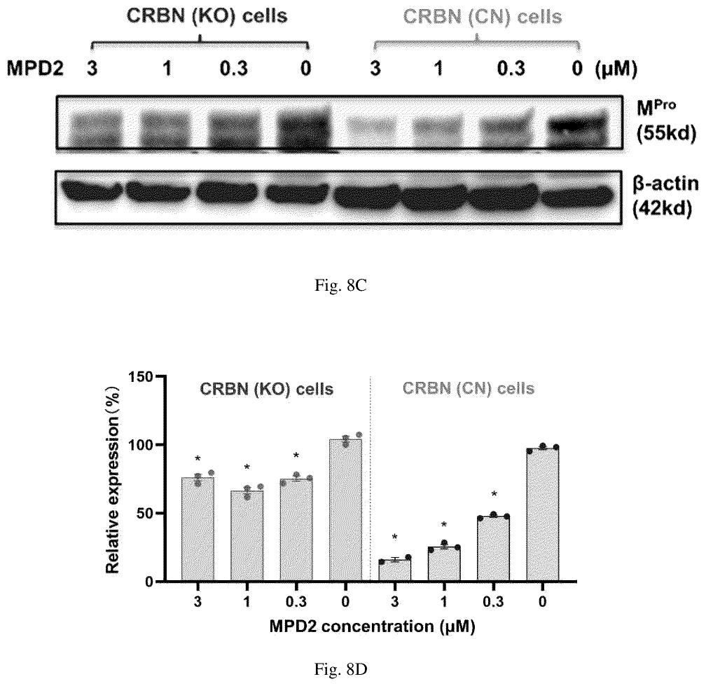

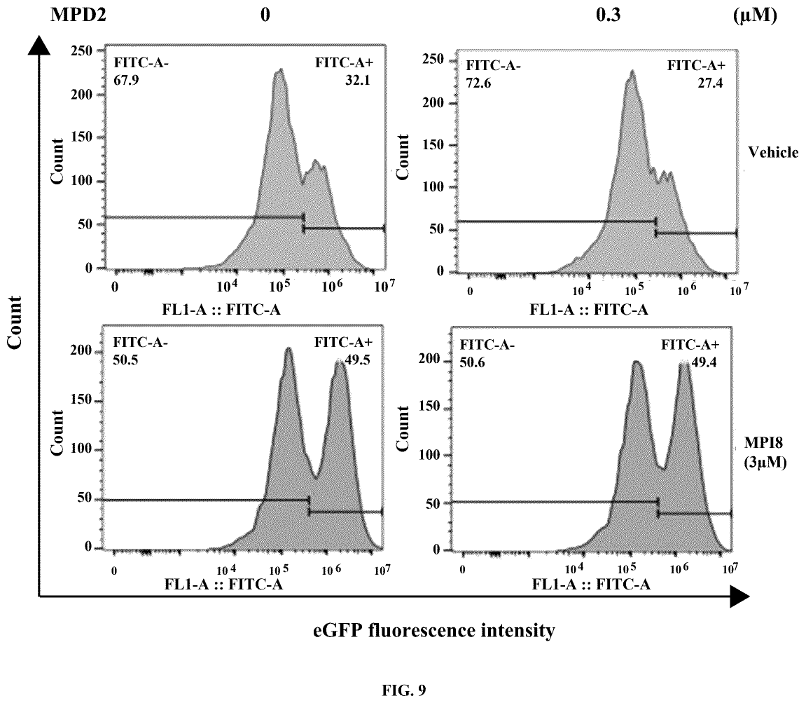

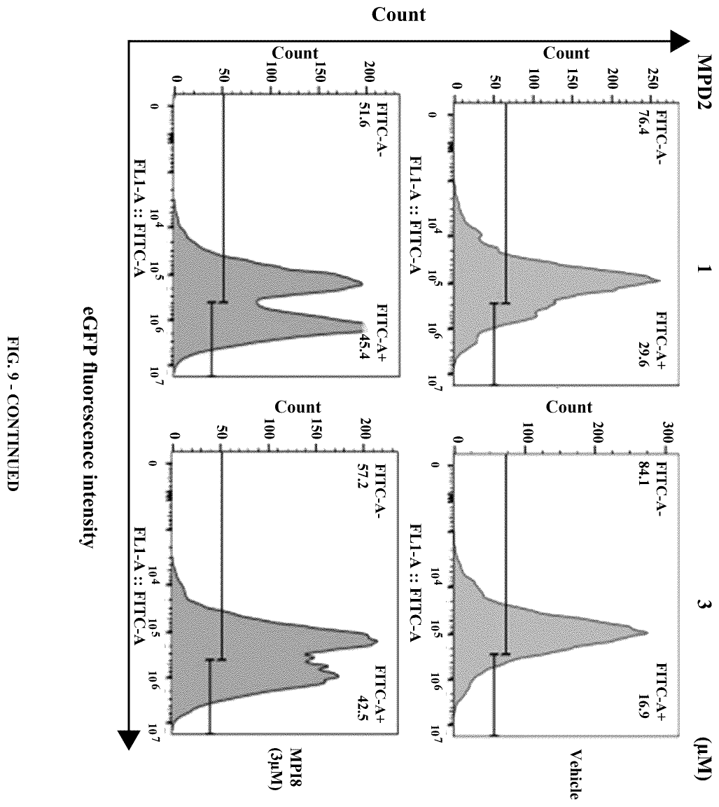

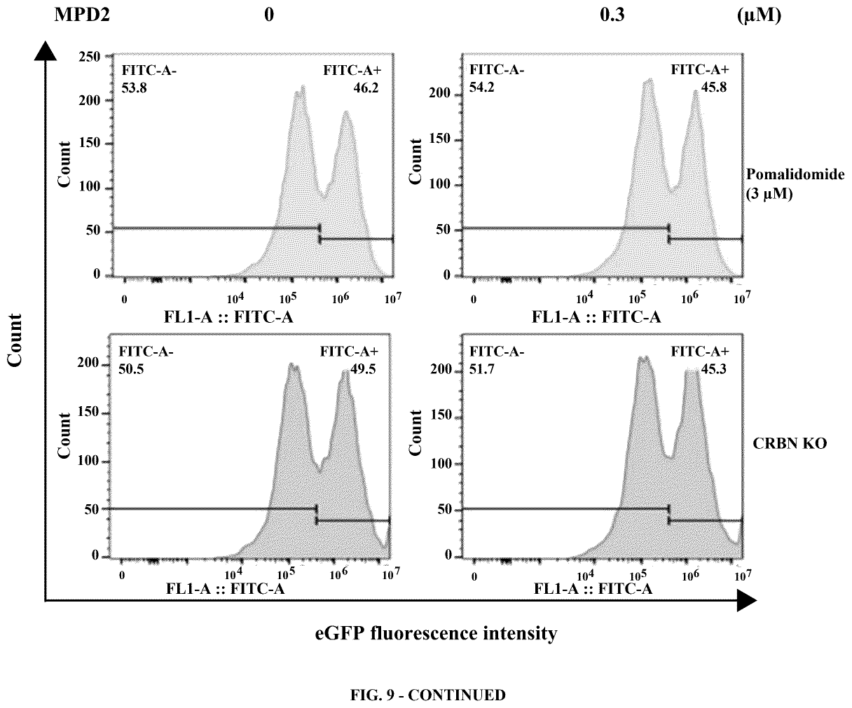

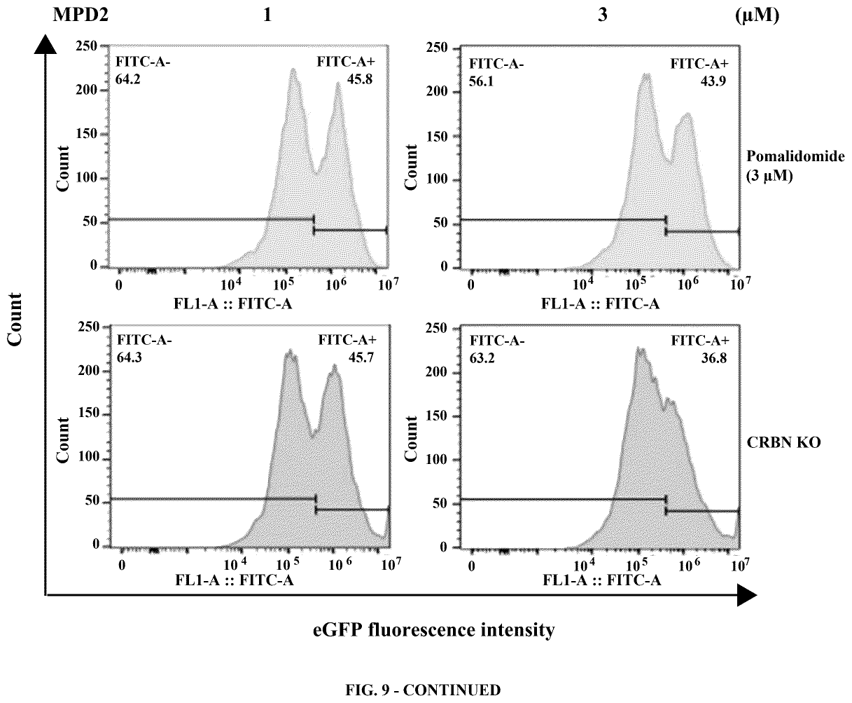

To validate the mechanism underlying M^{Pro} degradation depending on the concurrent presence of both M^{Pro} and CRBN binding, competition assay experiments were conducted. Initially, the pretreatment of the cells with 3 μM MPI8 (an M^{Pro} ligand) completely blocked the degradation of M^{Pro} by MPD2 (FIGS. 8A and 8B, and FIG. 9). Similarly, the pretreatment of the cells with 3 μM Pomalidomide (a CRBN ligand) resulted in the suppression of the M^{Pro} degradation by MPD2 (FIGS. 8A and 8B, and FIG. 9). These studies implied that the degradation of M^{Pro} induced by MPD2 depends on M^{Pro} binding and CRBN binding. To further establish the CRBN dependance of the degradation mechanism of MPD2, CRBN knockout (CRBN-KO) M^{Pro}-eGFP 293T cells were generated using CRISPR editing technique. When comparing the MPD2-induced degradation of M^{Pro} in wildtype CRBN (CN) and CRBN (KO) cells, a significant rescue of intracellular M^{Pro} in cells lacking CRBN was observed (FIGS. 8C and 8D, and FIG. 9). The results of the competition assays with MPI8 and pomalidomide, alongside the CRBN knockout study, collectively affirm that MPD2-induced M^{Pro} degradation relies on both M^{Pro} and CRBN engagement, elucidating a CRBN-mediated mode of action.

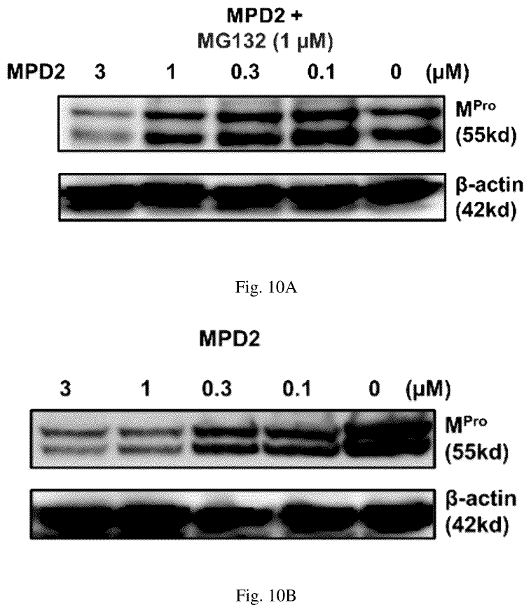

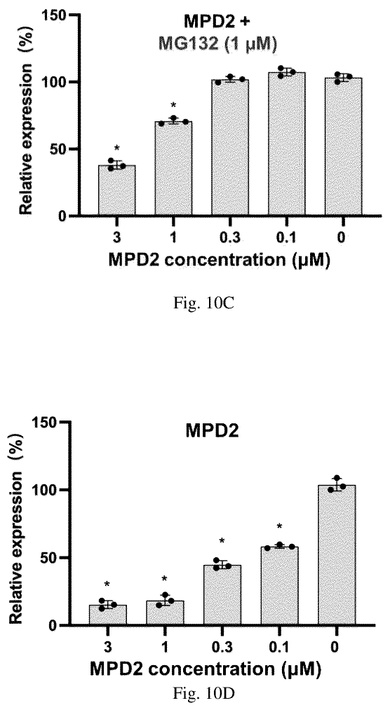

Moreover, it was observed that the proteasome inhibitor MG132 (1 μM) effectively rescued MPD2-induced M^{Pro} degradation. The notable increase in M^{Pro} levels resulting from the co-treatment of cells with MPD2 and MG132 unequivocally validates the indispensability of proteasome activity in the mechanistic degradation of MPD2 (FIGS. 10A-10D). It is essential to note that the presence of MG132 inhibits proteasome activity, thereby impeding the natural ubiquitination process of M^{Pro}, which subsequently leads to elevated M^{Pro} levels. Collectively, these studies utilizing CRBN knockout cells, in combination with MPI8, pomalidomide, MG132 and MPD2 treatments, provide compelling evidence that the degradation activity of MPD2 relies on the engagement of M^{Pro}, CRBN, and proteasome-mediated degradation. These findings underscore the multifaceted nature of M^{Pro} degradation induced by MPD2, requiring M^{Pro} binding, CRBN binding, and proteasome-mediated processes.

### Example 5: Antiviral Potency Characterizations

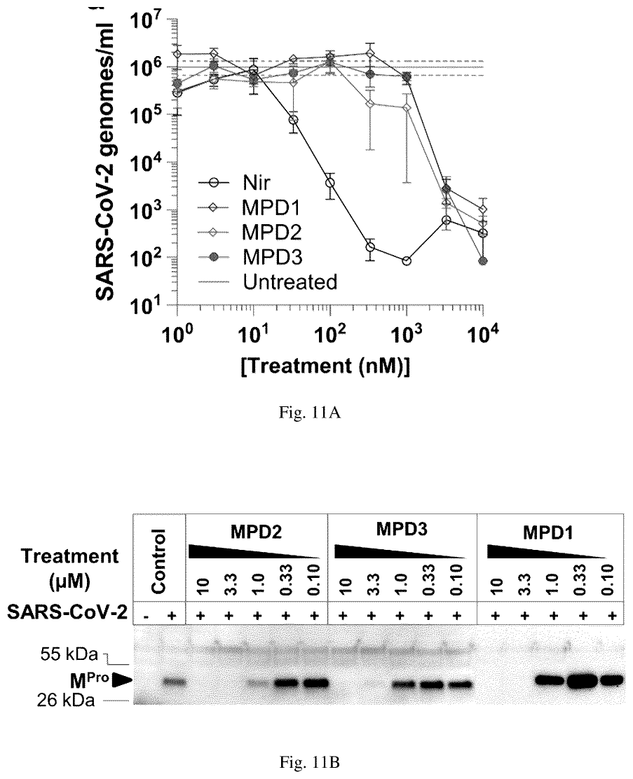

After successfully validating the degradation of M^{Pro} and understanding the mechanism of degradation induced by the PROTAC degraders, the next step was to evaluate their antiviral efficacy against SARS-CoV-2 in ACE2+A549 cells infected with hCoV-19/USA/HP05647/2021, an early Delta variant known for its robust growth in cell culture and strong cytopathic effects. MPD1-3 were administered at varying concentrations, and the cells were cultured for 72 h before quantifying SARS-CoV-2 mRNA levels using RT-PCR to determine the antiviral EC₅₀ values. MPD1, MPD2, and MPD3 exhibited antiviral EC₅₀ values of 1780 nM, 492 nM, and 1160 nM, respectively (FIG. 11A). Furthermore, the impact of PROTAC degrader treatment on M^{Pro} accumulation during SARS-CoV-2 infection was investigated. A549-ACE2 cells were inoculated at an approximate multiplicity of 0.1 and subsequently treated with MPD1-3, starting 1 h after inoculation. After 48 h post-inoculation, cell lysates were prepared and subjected to blotting using a monoclonal antibody against the 34 kDa M^{Pro}. All three degraders MPD1-3 demonstrated the ability to induce M^{Pro} degradation. Among them, MPD2 emerged as the most potent degrader, which correlates with its highest antiviral activity (FIG. 11B).

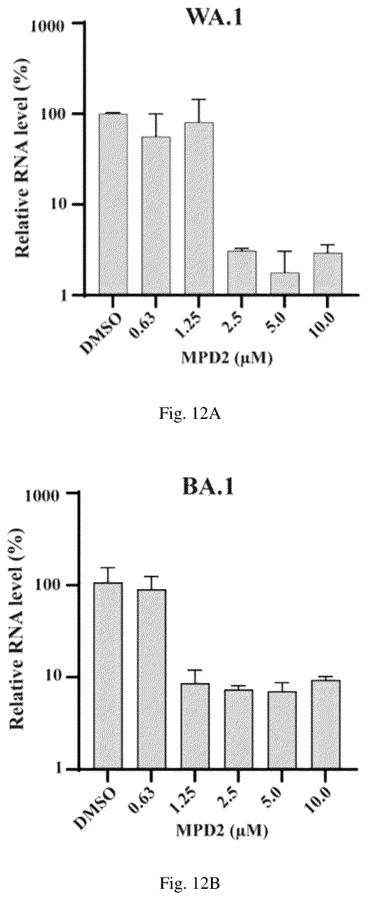

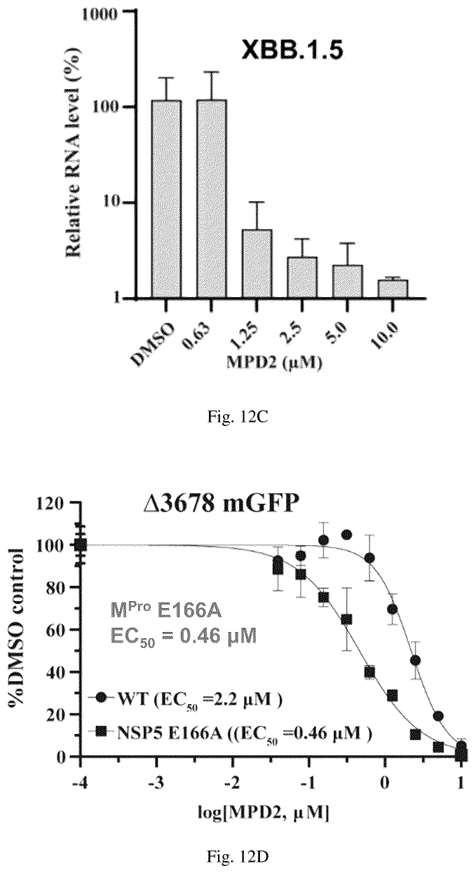

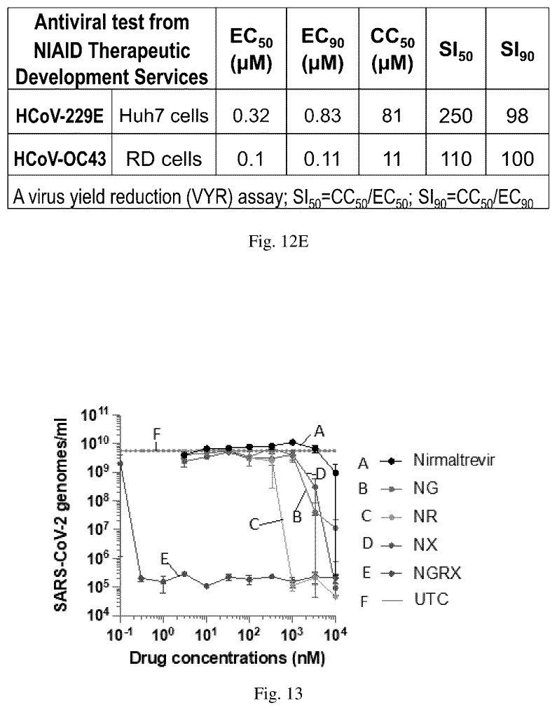

The antiviral activity of MPD2 against previous prevalent SARS-CoV-2 strains including WA.1, BA.1, and XBB.1.5 was further examined. At 2.5 μM concentration, MPD2 reduced viral production from infected A549-hACE2 cells by 90% across all three tested strains, demonstrating its efficacy against SARS-CoV-2 variants (FIGS. 12A-12C). Moreover, the effectiveness of MPD2 was assessed against a recombinant SARS-CoV-2 containing an NSP5 E166A mutation, known to confer a 10-fold resistance to nirmatrelvir treatment. This virus was engineered in the backbone of a live-attenuated BSL-2 SARS-CoV-2 Δ3678 mGFP. Notably, the results revealed that MPD2 exhibited 5-fold greater potency against the NSP5 E166A mutant virus compared to the wild-type Δ3678 mGFP (FIG. 12D). Collectively, the data underscore the significant potential of MPD2 in inhibiting SARS-CoV-2 variants and nirmatrelvir-resistant viruses.

### Example 6: MPD2 is a Broad-Spectrum Antiviral Against SARS-CoV-2 Variants, HCoV-229E, and HCoV-OC43

After successfully validating the mechanism of MPD2-induced MPro degradation, the antiviral activity of MPD2 was further examined against various SARS-CoV-2 variants. The recombinant variants of concerns were cloned, including BA.1, and XBB.1.5. A highly attenuated recombinant SARS-CoV-2 was also developed with a deletion of open-reading frames 3, 6, 7, 8, mutations of the transcription regulatory sequences and a green fluorescence reporter gene mNeonGreen (defined as Δ3678 mNG). This mutant virus is a valuable tool for rapidly evaluating the impact of MPro mutations on the antiviral activities of compounds. Using this system, the efficacy of MPD2 was assessed against the Δ3678 mNG with a nirmatrelvir-resistant mutation MPro E166A. The results in FIGS. 11A and 11B indicated that MPD2 effectively inhibits SARS-CoV-2 variants and nirmatrelvir-resistant mutant virus, E166A (EC50=460 nM). MPD2 has very low cytotoxicity with CC50 of 120 μM in 293T cells. In addition, potent antiviral activity of MPD2 against HCoV-229E (EC50=320 nM) and HCoV-OC43 (EC50=100 nM) was evaluated at the NIAID Therapeutic Development Services (FIG. 12E). Thus, MPD2 is an MPro PROTAC with potent antiviral activity against various SARS-CoV-2 strains, nirmatrelvir-resistant viruses, HCoV-229E and HCoV-OC43.

### Example 7: Discussion

The design, synthesis, and evaluation of bifunctional M^{Pro} PROTAC degraders MPD1-MPD10 based on reversible covalent M^{Pro} ligands MPI8 and MPI29 was explored. The CRBN-binding moieties of MPD1-MPD10 were derived from the well-established ligands thalidomide-4-OH and pomalidomide. Notably, all MPD1-MPD10 compounds exhibited sub-micromolar IC₅₀ values, underscoring the absence of any discernible interference from the linkers and CRBN E3 ligands with M^{Pro} binding (FIGS. 3A and 3B). The M^{Pro} degradation by PROTAC degraders MPD1-MPD3 was determined by a cellular system utilizing the M^{Pro}-eGFP fusion protein and Western blot analysis (FIGS. 4A-4H). These studies revealed that MPD2 effectively facilitated M^{Pro} degradation in M^{Pro}-eGFP 293T stable cells, characterized by rapid and enduring effects. To substantiate the degradation mechanism of the M^{Pro} degrader MPD2, a series of control experiments were conducted. Notably, pretreatment with an M^{Pro} inhibitor MPI8 (as a competitor for M^{Pro} binding), or a CRBN E3 ligand Pomalidomide (as a competitor of CRBN ligase binding), led to the suppression of M^{Pro} degradation induced by MPD2. These findings strongly implied that MPD2-induced M^{Pro} degradation hinges on both M^{Pro} and CRBN binding. Furthermore, studies involving CRBN knockout cells demonstrated a significant reduction in the degradation potency of MPD2, further affirming a CRBN-dependent mechanism (FIGS. 8A-8D). The investigation also unveiled that the proteasome inhibitor MG132 could rescue the degradation triggered by MPD2, underscoring the dependence of MPD2's activity on proteasome-mediated degradation (FIGS. 10A-10D). Taken together, MPD2 has proven to be a potent and effective M^{Pro} degrader that relies on a multifaceted mechanism encompassing M^{Pro} binding, CRBN binding, and proteasome-mediated degradation. Importantly, MPD2 exhibited remarkable antiviral activity against various SARS-CoV-2 variants, and successfully degraded M^{Pro} in SARS-CoV-2-infected A549-ACE2 cells (FIGS. 11A, 11B, and 12A-12E). Notably, MPD2 exhibited 5-fold greater potency against the NSP5 E166A mutant nirmatrelvir-resistant virus compared to the wild-type Δ3678 mGFP (FIG. 12D). Collectively, these data underscore the significant potential of MPD2 in inhibiting SARS-CoV-2 variants and nirmatrelvir-resistant viruses (FIG. 12C).

In summary, a series of SARS-CoV-2 M^{Pro} degraders was developed, with MPD2 emerging as a standout lead compound. MPD2 effectively reduced M^{Pro} protein levels in 293T cells, relying on a time-dependent, CRBN-mediated, and proteasome-driven mechanism. Furthermore, MPD2 exhibited remarkable efficacy in diminishing M^{Pro} protein levels in SARS-CoV-2-infected A549-ACE2 cells. MPD2 also displayed potent antiviral activity against various SARS-CoV-2 strains and exhibited enhanced potency against nirmatrelvir-resistant viruses. This study highlights the potential of leveraging PROTAC-mediated targeted protein degradation as an approach to target M^{Pro} for developing antivirals that could fight against drug-resistant viral variants.

### Example 8: Enhancement of Approved COVID Therapeutics by PROTACs

The synergy of PROTACs with nirmatrelvir and ritonavir, the components of the approved anti-COVID treatment Paxlovid was explored. These results show SARS-CoV-2 (strain XBB.1.5) growing in Vero-ACE2-TMPRSS2 cells. The left axis shows the amount of virus growth, measured in genome equivalents per ml by quantitative PCR. The bottom axis shows the amount of nirmatrelvir.

Variable amounts of nirmatrelvir (abbreviated “N” on the figure) were tested with and without fixed amounts of 10 μM MPD2 (coronavirus main protease degrader PROTAC, called “X” on the figure), 10 μM GRL-0617 (coronavirus papain-like protease degrader PROTAC, called “G” on the figure), 5 μM ritonavir (an enhancer that is already part of Paxlovid, called “R” on the figure). For example, NR shows the effect of a range of nirmatrelvir “N” concentrations indicated on the bottom axis, with 5 μM ritonavir “R”. Untreated control (UTC) shows the amount of virus growth without N, G, R or X present.

FIG. 13 shows the effects of nirmatrelvir alone (N), nirmatrelvir in combination with each other component (NX, NG, and NR respectively), and all four treatments together (NGRX). For clarity, the NGRX line shows the effect of the amount of nirmatrelvir indicated on the lower axis with 10 μM MPD2, 10 μM GRL-0617 and 5 μM ritonavir together.

It can be seen that each component shifts the original effectiveness curve to the left, meaning the same effect was achieved, but using less nirmatrelvir. There is a small shift for each individual enhancer (G, R and X), and an enormous shift for the combination of N, G, R and X. This is based on 4 replicates per point on the graph, and has been repeated three times (including once with the Delta variant of SARS-CoV-2 in A549-ACE2 cells and twice with XBB.1.5 variant in Vero-ACE2-TMPRSS2 cells.)

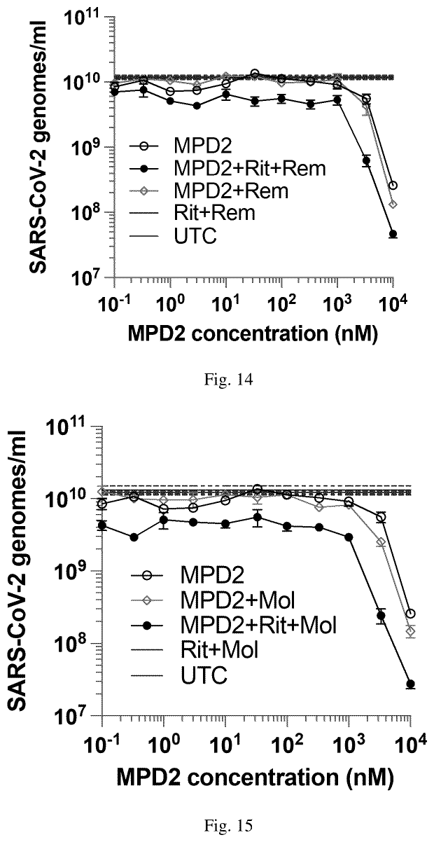

FIG. 14 shows the synergistic effect of MPD2 PROTAC on the approved COVID antiviral Remdesivir. A variable amount of MPD2 shown in the lower axis (MPD2) was tested with and without fixed concentration of 4.4 μM remdesivir (Rem) and/or 5 μM ritonavir (Rit). Untreated controls (UTC) and Rem-Rit controls are shown. Again, the addition of MPD2 increased the effectiveness of a SARS-CoV-2 antiviral—in this case, Remdesivir.

FIG. 15 shows the results of a similar experiment to FIG. 14, but with the approved SARS-CoV-2 antiviral Molnupiravir replacing Remdesivir. The effects of a fixed amount of Molnupiravir (4.4 μM, “Mol” on FIG. 4) and Ritonavir (5 μM, “Rit” on FIG. 4) are shown; other markings are as shown in FIG. 14. This shows that addition of MPD2 increases the effectiveness of a third SARS-CoV-2 antiviral, Molnupiravir.

Molnupiravir, Remdesivir and Paxlovid (nirmatrelvir plus ritonavir) are the only currently approved antiviral therapeutics for use against SARS-CoV-2. Therefore, PROTACs GRL-0617 and MPD2 enhance the activity of all currently approved SARS-CoV-2 antiviral drugs.

What is claimed is:

1. A conjugate having a formula Q-L-P, wherein
Q is a radical of a ubiquitin ligase ligand;
L is a divalent linker; and
P is a radical of a SARS-CoV-2 protease ligand.

2. The conjugate of claim 1, wherein the protease ligand (P) is of Formula I: 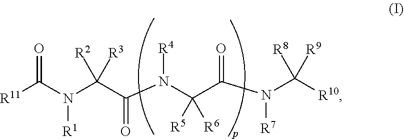
or a pharmaceutically acceptable salt thereof, wherein
each of R¹, R⁴, and R⁷ is independently H, C₁-C₆ alkyl, C₂-C₆ alkenyl, C₃-C₈ cycloalkyl, C₆-C₁₀ aryl, or 5- to 10-membered heteroaryl;
R² is C₁-C₆ alkylene-R^{A}, C₁-C₆ alkyl, C₂-C₆ alkenyl, C₃-C₈ cycloalkyl, 3- to 12-membered heterocycloalkyl, C₆-C₁₀ aryl, 5- to 10-membered heteroaryl, C₁-C₆ alkylene-C₆-C₁₀ aryl, or C₁-C₆ alkylene-5- to 10-membered heteroaryl, wherein each hydrogen atom in C₁-C₆ alkyl, C₁-C₆ alkylene, C₂-C₆ alkenyl, C₃-C₈ cycloalkyl, 3- to 12-membered heterocycloalkyl, C₆-C₁₀ aryl, or 5- to 10-membered heteroaryl is optionally substituted by R^{a};
each of R⁵ and R⁸ is independently C₁-C₆ alkyl, C₂-C₆ alkenyl, C₃-C₈ cycloalkyl, 3- to 12-membered heterocycloalkyl, C₆-C₁₀ aryl, 5- to 10-membered heteroaryl, C₁-C₆ alkylene-C₆-C₁₀ aryl, or C₁-C₆ alkylene-5- to 10-membered heteroaryl, wherein each hydrogen atom in C₁-C₆ alkyl, C₁-C₆ alkylene, C₂-C₆ alkenyl, C₃-C₅ cycloalkyl, 3- to 12-membered heterocycloalkyl, C₆-C₁₀ aryl, or 5- to 10-membered heteroaryl is optionally substituted by R^{a}; or R⁴ and R⁵ together with the atoms to which they are attached combine to form a 5- to 8-membered heterocycloalkyl, wherein each hydrogen atom in 5- to 8-membered heterocycloalkyl is optionally substituted by R^{c};
each of R³, R⁶, and R⁹ is H;
R¹⁰ is C₁-C₆ alkyl-SO₃H, C₁-C₆ alkyl-CN, —C(O)H, —C(O)C₁-C₆ alkyl, —C(O)C(O)NR^{b}R^{b}, —C(O)COC(O)C₁-C₁₀ alkyl, —C(O)COC(O)C₆-C₁₀ aryl, —C(O)COC(O)-5- to 10-membered heteroaryl, or —CN, wherein each hydrogen atom in C₁-C₆ alkyl, and C₆-C₁₀ aryl is optionally substituted by halo, —OSO₃H, hydroxy, OC₁—C₆ alkyl, —CN, —NO₂, C₆-C₁₀ aryl, or —NR^{b}R^{b};
R¹¹ is R^{A}, —O—C₁-C₆ alkyl, —O—C₆-C₁₀ aryl, —O-5- to 10-membered heteroaryl, —O—C₁-C₆ alkylene-C₆-C₁₀ aryl, or —O—C₁-C₆ alkylene-5- to 10-membered heteroaryl, wherein C₁-C₆ alkyl, C₁-C₆ alkylene, C₆-C₁₀ aryl, or 5- to 10-membered heteroaryl is optionally substituted by R^{a};
R^{A} is 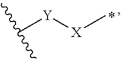
wherein [symbol] represents a point of attachment to the divalent linker (L), and * represents a point of attachment to P;
X is absent, NH, O, or S;
Y is a bond, C₁-C₆ alkylene, C₆-C₁₀ arylene, 5- to 10-membered heteroarylene, C₁-C₆ alkylene-C₆-C₁₀ arylene, or C₁-C₆ alkylene-5- to 10-membered heteroarylene;
each R^{a}, when present, is independently halo, hydroxy, C₁-C₆ alkyl, —O—C₁-C₆ alkyl, C₃-C₈ cycloalkyl, or 3- to 12-membered heterocycloalkyl;
each R^{b}, when present, is independently H, C₁-C₆ alkyl, C₂-C₆ alkenyl, C₃-C₈ cycloalkyl, or 3- to 12-membered heterocycloalkyl, wherein each hydrogen atom in C₁-C₆ alkyl, C₂-C₆ alkenyl, C₃-C₈ cycloalkyl, or 3- to 12-membered heterocycloalkyl is optionally substituted by R^{a};
each R^{c}, when present, is halo, hydroxy, C₁-C₆ alkyl, —O—C₁-C₆ alkyl, or C₃-C₈ cycloalkyl; or two R^{c} combine together with the atom or atoms to which they are attached to form a C₃-C₈ cycloalkyl, wherein each hydrogen atom in C₃-C₈ cycloalkyl is optionally substituted by R^{a}; and
p is 0 or 1;
provided that when R¹¹ is R^{A}, then R² is not alkylene-R^{A}.

3. The conjugate of claim 2, wherein the protease ligand (P) is of Formula Ia: 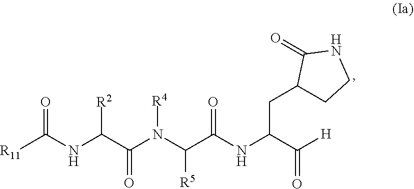
or a pharmaceutically acceptable salt thereof, wherein
R² is C₁-C₆ alkylene-R^{A}, C₁-C₆ alkyl, C₂-C₆ alkenyl, C₃-C₈ cycloalkyl, or 3- to 12-membered heterocycloalkyl, wherein each hydrogen atom in C₁-C₆ alkyl, C₂-C₆ alkenyl, C₃-C₈ cycloalkyl, or 3- to 12-membered heterocycloalkyl is optionally substituted by R^{a};
R⁴ is H;
R⁵ is C₁-C₆ alkyl, C₂-C₆ alkenyl, C₃-C₈ cycloalkyl, or 3- to 12-membered heterocycloalkyl, wherein each hydrogen atom in C₁-C₆ alkyl, C₂-C₆ alkenyl, C₃-C₈ cycloalkyl, or 3- to 12-membered heterocycloalkyl, is optionally substituted by R^{a}; or R⁴ and R⁵ together with the atoms to which they are attached combine to form a 5- to 8-membered heterocycloalkyl wherein each hydrogen atom in 5- to 8-membered heterocycloalkyl is optionally substituted by R^{c};
R¹¹ is R^{A}, —O—C₁-C₆ alkyl, —O—C₆-C₁₀ aryl, —O-5- to 10-membered heteroaryl, —O—C₁-C₆ alkylene-C₆-C₁₀ aryl, or —O—C₁-C₆ alkylene-5- to 10-membered heteroaryl, wherein C₁-C₆ alkyl, C₁-C₆ alkylene, C₆-C₁₀ aryl, or 5- to 10-membered heteroaryl is optionally substituted by R^{a};
R^{A} is 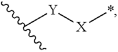
wherein [symbol] represents a point of attachment to the divalent linker (L), and * represents a point of attachment to P;
X is absent, NH, O, or S;
Y is a bond, C₁-C₆ alkylene, C₆-C₁₀ arylene, 5- to 10-membered heteroarylene, C₁-C₆ alkylene-C₆-C₁₀ arylene, or C₁-C₆ alkylene-5- to 10-membered heteroarylene;
each R^{a}, when present, is independently halo, hydroxy, C₁-C₆ alkyl, —O—C₁-C₆ alkyl, C₃-C₈ cycloalkyl, 3- to 12-membered heterocycloalkyl, C₆-C₁₀ aryl, or 5- to 10-membered heteroaryl; and
each R^{c}, when present, is halo, hydroxy, C₁-C₆ alkyl, OC₁—C₆ alkyl, or C₃-C₈ cycloalkyl; or two R^{c} combine together with the atom or atoms to which they are attached to form a C₃-C₈ cycloalkyl, wherein each hydrogen atom in C₃-C₈ cycloalkyl is optionally substituted by R^{a};
provided that when R¹¹ is R^{A}, then R² is not alkylene-R^{A}.

4. The conjugate of claim 3, wherein L is selected from the group consisting of alkylene, alkenylene, alkynylene, arylene, heteroarylene, ether, amino, amide, carbamate, carbonate, ester, ketone, sulfate, sulfonamide, sulfoxide, sulfone, sulfonate, thioester, thioether, alkoxy, urea, hydrazine, and any combination thereof.

5. The conjugate of claim 3, wherein Y is selected from the group consisting of a bond, 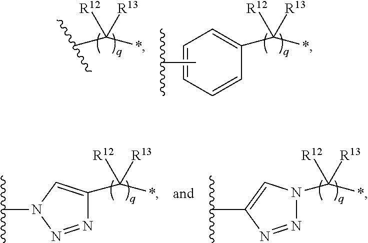
wherein each of R¹² and R¹³ is independently H, halo, hydroxy, C₁-C₆ alkyl, OC₁—C₆ alkyl, C₂-C₆ alkenyl, or C₃-C₈ cycloalkyl; and
q is 1, 2, 3, 4, 5, or 6; and
wherein [symbol] represents a point of attachment of P to the divalent linker (L), and * represents a point of attachment to P.

6. The conjugate of claim 5, wherein R² is C₁-C₆ alkyl, C₂-C₆ alkenyl, C₃-C₈ cycloalkyl, or 3- to 12-membered heterocycloalkyl, wherein each hydrogen atom in C₁-C₆ alkyl, C₂-C₆ alkenyl, C₃-C₈ cycloalkyl, 3- to 12-membered heterocycloalkyl, is optionally substituted by R^{a}.

7. The conjugate of claim 6, wherein R² is optionally substituted C₁-C₆ alkyl.

8. The conjugate of claim 7, wherein R² is

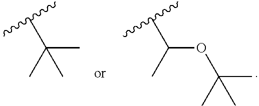

9. The conjugate of claim 8, wherein R⁵ is optionally substituted C₁-C₆ alkyl.

10. The conjugate of claim 9, wherein R⁵ is

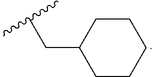 [SMILES: *CC1CCCCC1]

11. The conjugate of claim 8, wherein R⁴ and R⁵ together with the atoms to which they are attached combine to form 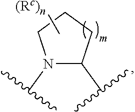
wherein n is 0, 1, or 2, and m is 1, 2, 3, or 4.

12. The conjugate of claim 11, wherein R⁴ and R⁵ together with the atoms to which they are attached combine to form

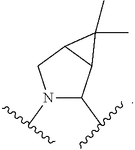 [SMILES: *C1C2C(CN1*)C2(C)C]

13. The conjugate of claim 12, wherein R⁸ is

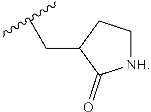 [SMILES: *CC1CCNC1=O]

14. The conjugate of claim 1, wherein the protease ligand (P) is selected from the group consisting of 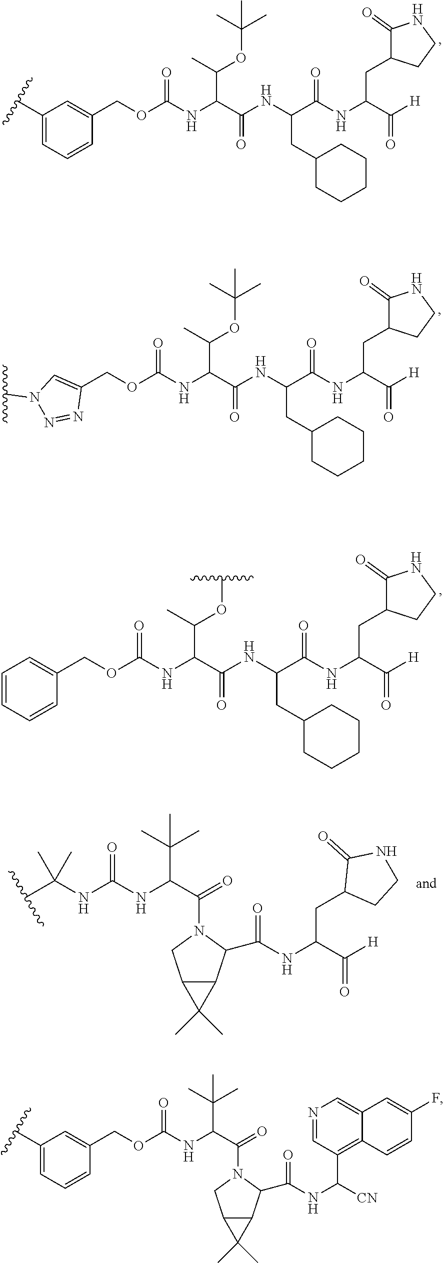
or a pharmaceutically acceptable salt thereof;
wherein “ [symbol] ” represents a point of attachment to the divalent linker (L).

15. The conjugate of claim 1, wherein the ubiquitin ligase ligand (Q) is of Formula II 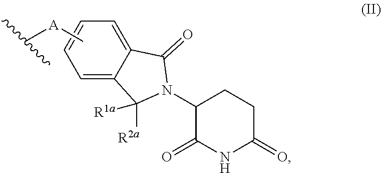
or a pharmaceutically acceptable salt thereof, wherein
A is ether, amino, amide, carbamate, carbonate, ester, ketone, sulfate, sulfonamide, sulfoxide, sulfone, sulfonate, thioester, thioether, alkoxy, urea, hydrazine, or aminoalkyl; and
each of R^{1a} and R^{2a} is independently H or C₁-C₆ alkyl; or R^{1a} and R^{2a} come together on the carbon atom to which they are attached to form an oxo;
wherein “ [symbol] ” represents a point of attachment to the divalent linker (L).

16. The conjugate of claim 1, wherein the ubiquitin ligase ligand (Q) is of Formula III: 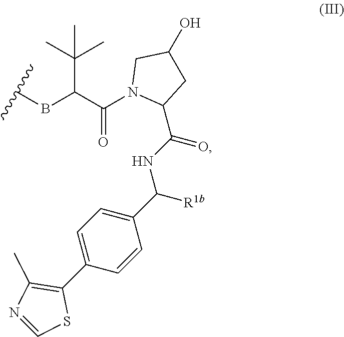
or a pharmaceutically acceptable salt thereof, wherein
B is ether, amino, amide, carbamate, carbonate, ester, ketone, sulfate, sulfonamide, sulfoxide, sulfone, sulfonate, thioester, thioether, alkoxy, urea, hydrazine, or aminoalkyl; and
R^{1b} is H or C₁-C₆ alkyl;
wherein “ [symbol] ” represents a point of attachment to the divalent linker (L).

17. The conjugate of claim 1, wherein the ubiquitin ligase ligand (Q) is 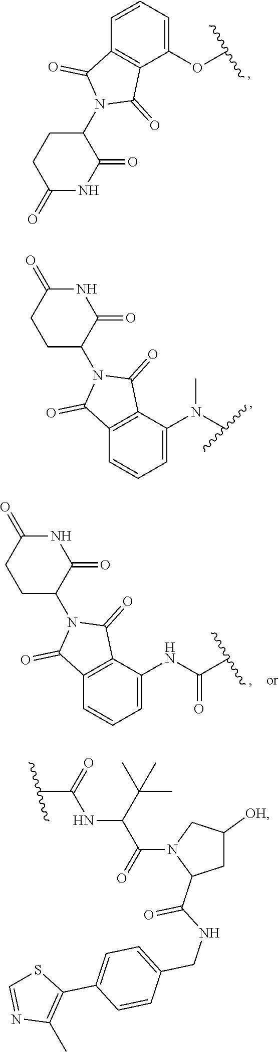
or a pharmaceutically acceptable salt thereof,
wherein “ [symbol] ” represents a point of attachment to the divalent linker (L).

18. The conjugate of claim 1, selected from the group consisting of: 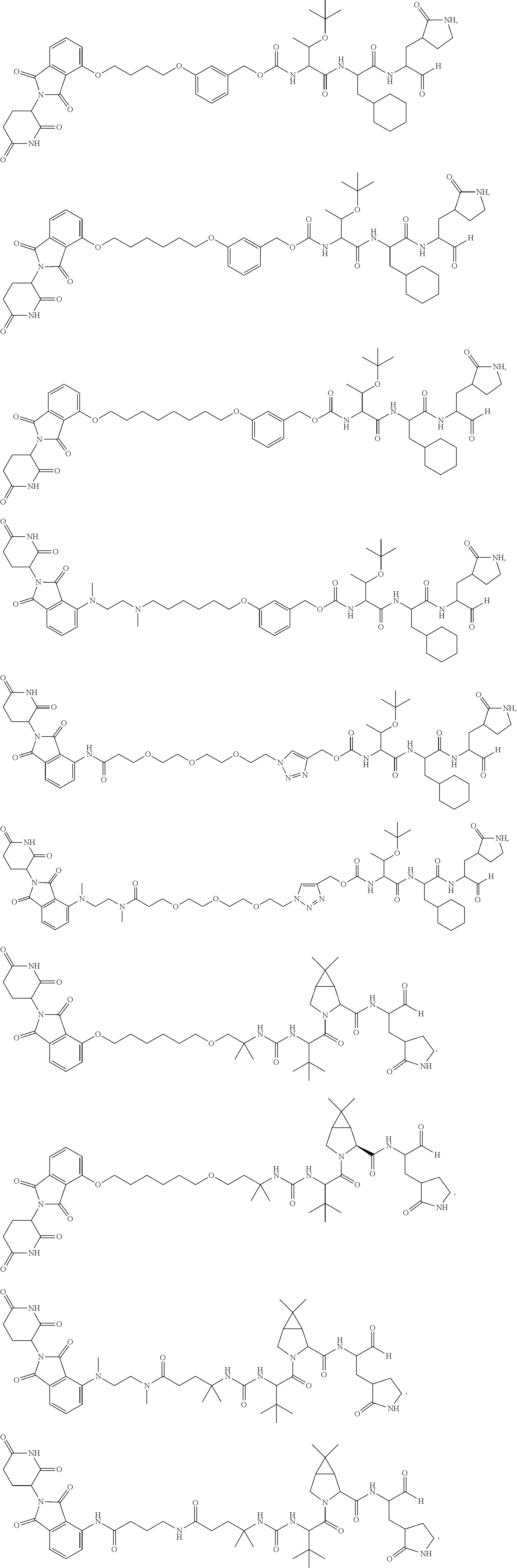 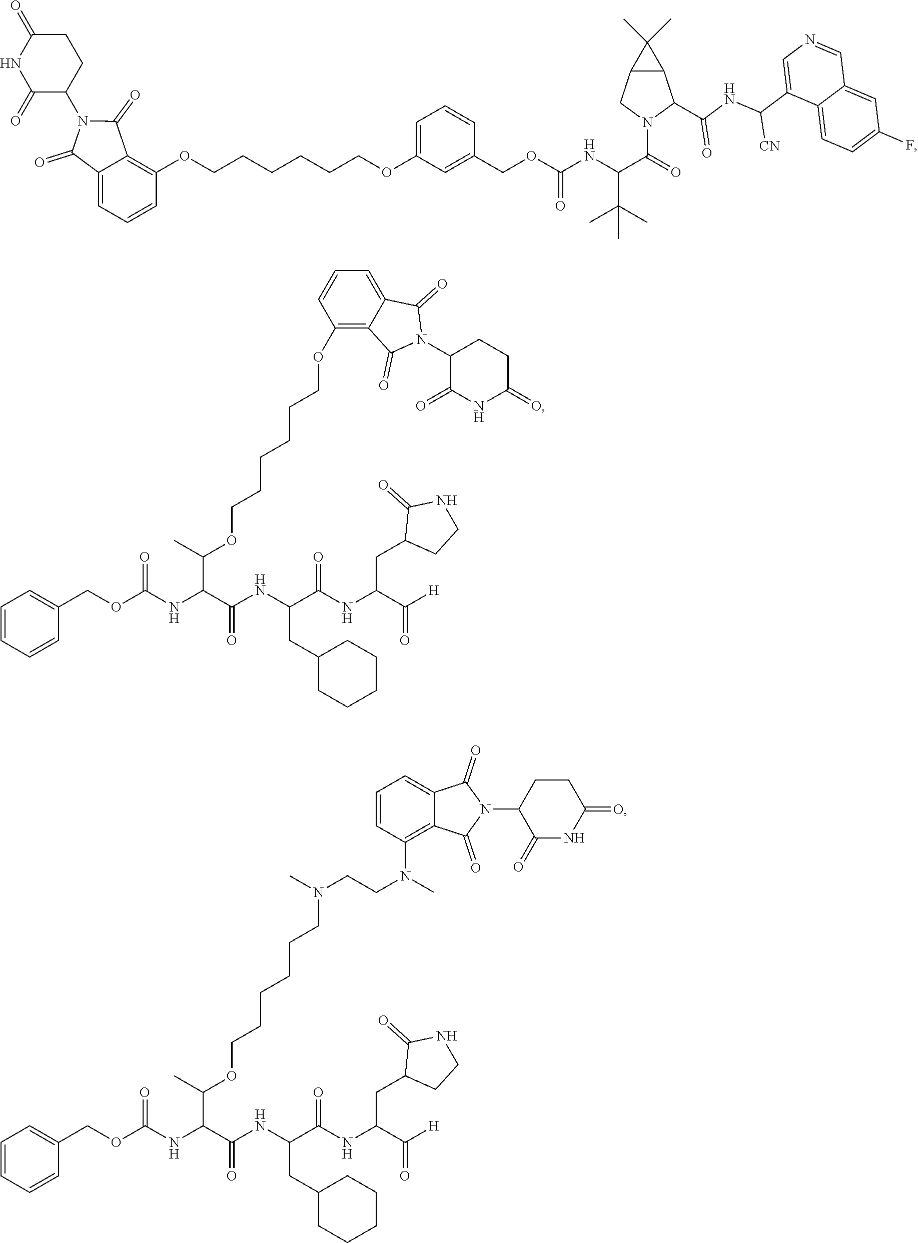 [SMILES: CC(C)(C)C(NC(=O)OCc1cccc(OCCCCCCOc2cccc3c2C(=O)N(C2CCC(=O)NC2=O)C3=O)c1)C(=O)N1CC2C(C1C(=O)NC(C#N)c1cncc3cc(F)ccc13)C2(C)C ; CC(OCCCCCCOc1cccc2c1C(=O)N(C1CCC(=O)NC1=O)C2=O)C(NC(=O)OCc1ccccc1)C(=O)NC(CC1CCCCC1)C(=O)NC(C=O)CC1CCNC1=O ; CC(OCCCCCCN(C)CCN(C)c1cccc2c1C(=O)N(C1CCC(=O)NC1=O)C2=O)C(NC(=O)OCc1ccccc1)C(=O)NC(CC1CCCCC1)C(=O)NC(C=O)CC1CCNC1=O]
or a pharmaceutically acceptable salt thereof.

19. A pharmaceutical composition comprising at least one compound of claim 1, or a pharmaceutically acceptable salt thereof, and optionally one or more pharmaceutically acceptable excipients.

20. A method of treating disease, such as a viral infection, comprising administering to a subject in need of such treatment an effective amount of a compound of claim 1, or a pharmaceutically acceptable salt thereof.


SEQUENCE LISTING (7 sequences)

SEQ ID NO: 1 - DNA, 20 bp, synthetic construct
  source 1..20: mol_type = other DNA
gaccccaaaatcagcgaaat

SEQ ID NO: 2 - DNA, 24 bp, synthetic construct
  source 1..24: mol_type = other DNA
tctggttactgccagttgaatctg

SEQ ID NO: 3 - DNA, 24 bp, synthetic construct
  source 1..24: mol_type = other DNA
accccgcattacgtttggtggacc

SEQ ID NO: 4 - DNA, 20 bp, synthetic construct
  source 1..20: mol_type = other DNA
ttacaaacattggccgcaaa

SEQ ID NO: 5 - DNA, 18 bp, synthetic construct
  source 1..18: mol_type = other DNA
gcgcgacattccgaagaa

SEQ ID NO: 6 - protein, 14 aa, synthetic construct
KTSAVLQSGFRKME

SEQ ID NO: 7 - protein, 5 aa, synthetic construct
MAVLQ